(19) United States
(12) Patent Application Publication
(10) Pub. No.: US 2024/0150351 A1
(43) Pub. Date: May 9, 2024

(54) BICYCLIC COMPOUNDS

(71) Applicant: Aligos Therapeutics, Inc., South San Francisco, CA (US)

(72) Inventors: Sandrine Vendeville, Brussels (BE); David Bernard Smith, San Mateo, CA (US); Qingling Zhang, Emerald Hills, CA (US); Yannick Debing, Bilzen (BE)

(21) Appl. No.: 18/476,775

(22) Filed: Sep. 28, 2023

Related U.S. Application Data
(60) Provisional application No. 63/377,940, filed on Sep. 30, 2022.

(51) Int. Cl. C07D 471/04; A61K 31/519; A61K 31/675; A61K 45/06; A61P 31/14; C07F 9/6561

(52) U.S. Cl. CPC C07D 471/04; A61K 31/519; A61K 31/675; A61K 45/06; A61P 31/14; C07F 9/6561

(57) ABSTRACT

Provided herein are compounds of Formula (I), or pharmaceutically acceptable salts thereof, pharmaceutical compositions that include a compound described herein (including pharmaceutically acceptable salts of a compound described herein) and methods of synthesizing the same. Also provided herein are methods of treating diseases and/or conditions with a compound of Formula (I), or a pharmaceutically acceptable salt thereof.

### INCORPORATION BY REFERENCE TO ANY PRIORITY APPLICATIONS

Any and all applications for which a foreign or domestic priority claim is identified in the Application Data Sheet as filed with the present application are hereby expressly incorporated by reference under 37 CFR 1.57, and Rules 4.18 and 20.6, including U.S. Provisional Application No. 63/377,940, filed Sep. 30, 2022, which is hereby incorporated by reference in its entirety.

### REFERENCE TO SEQUENCE LISTING

The present application is filed with a Sequence Listing in Electronic format. The Sequence Listing is provided as a file entitled ALIG089.xml, created Sep. 26, 2023, which is approximately 6,520 bytes in size. The information in the electronic format of the sequence listing is incorporated herein by reference in its entirety.

### BACKGROUND

### Field

The present application relates to the fields of chemistry, biochemistry and medicine. Disclosed herein are compounds of Formula (I), or pharmaceutically acceptable salt thereof, pharmaceutical compositions that include a compound described herein (including pharmaceutically acceptable salts of a compound described herein) and methods of synthesizing the same. Also disclosed herein are methods of treating diseases and/or conditions with a compound of Formula (I), or a pharmaceutically acceptable salt thereof.

### Description

The hepatitis B virus (HBV) is a DNA virus and a member of the Hepadnaviridae family. HBV infects more than 300 million worldwide, and is a causative agent of liver cancer and liver disease such as chronic hepatitis, cirrhosis, and hepatocellular carcinoma. Although there are approved drugs for treating HBV, by either boosting the immune system or slowing down the replication of the HBV virus, HBV continues to be a problem due to the drawbacks associated with each of the approved drugs and the very low rates of functional cure achieved with these treatments.

### SUMMARY

Some embodiments disclosed herein relate to a compound of Formula (I), or a pharmaceutically acceptable salt thereof.

Some embodiments disclosed herein relate to a pharmaceutical composition that can contain an effective amount of a compound of Formula (I), or a pharmaceutically acceptable salt thereof.

Some embodiments described herein relate to a method of treating a HBV and/or HDV infection that can include administering to a subject identified as suffering from the HBV and/or HDV infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a HBV and/or HDV infection.

Some embodiments disclosed herein relate to a method of inhibiting replication of HBV and/or HDV that can include contacting a cell infected with the HBV and/or HDV with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication HBV and/or HDV.

These are other embodiments are described in greater detail below.

### DETAILED DESCRIPTION

HBV is a partially double-stranded circular DNA of about 3.2 kilobase (kb) pairs, and is classified into at least eight genotypes. The HBV replication pathway has been studied in great detail. T. J. Liang, Hepatology (2009) 49(5 Suppl):S13-S21. One part of replication includes the formation of the covalently closed circular DNA (cccDNA) form. The presence of the cccDNA gives rise to the risk of viral reemergence throughout the life of the host. HBV carriers can transmit the disease for many years. An estimated 300 million people are living with chronic hepatitis B, and it is estimated that over 750,000 people worldwide die of hepatitis B each year. In addition, immunosuppressed individuals or individuals undergoing chemotherapy are especially at risk for reactivation of a HBV infection.

HBV can be transmitted by blood, semen, and/or another body fluid. This can occur through direct blood-to-blood contact, unprotected sex, sharing of needles, but most often transmission occurs from an infected mother to her baby during the delivery process. The HBV surface antigen (HBsAg) is most frequently used to screen for the presence of this infection. Currently available medications rarely cure HBV infection. Rather, the medications suppress replication of the virus.

The hepatitis delta virus (HDV) is an RNA virus. HDV can propagate only in the presence of an established HBV infection. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all viral hepatitis forms, at 20%.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3) individually and independently selected from deuterium, alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroalkyl, hydroxy, alkoxyalkyl, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, C-amido(alkyl), isocyanato, thiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amine, a di-substituted amine, an unsubstituted C-amido(C₁₋₃ alkyl), —O-(an unsubstituted C₁₋₄ alkyl)-OH, —O-(an unsubstituted C₁₋₄ alkyl)-(an unsubstituted alkoxy), —O-(an unsubstituted C₁₋₄ alkyl)-(an unsubstituted C-carboxy), —O—(C₁₋₃ alkyl)-O-(an unsubstituted C-amido), —O-(an unsubstituted C₁₋₄ alkyl)-NH₂, —O-(an unsubstituted C₁₋₄ alkyl)-NH(an unsubstituted C₁₋₄ alkyl), —O-(an unsubstituted C₁₋₄ alkyl)-N(an unsubstituted C₁₋₄ alkyl)₂ and an unsubstituted —O-(an unsubstituted C₁₋₄ alkyl)-CN.

As used herein, “Cₐ to C_{b}”, “Cₐ-C_{b}” or “C_{a-b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl”, “C₁-C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. Byway of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl groups may be unsubstituted or substituted. Examples of such “heterocyclyl groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and 3,4-methylenedioxyphenyl).

As used herein, “aryl(alkyl)” refers to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

A “(heterocyclyl)alkyl” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted.” Further, when a lower alkylene group is substituted, the lower alkylene can be substituted by replacing both hydrogens on the same carbon with a cycloalkyl group

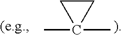

As used herein, “alkoxy” refers to the formula —OR wherein R is an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. In some instances, an alkoxy can be —OR, wherein R is an unsubstituted C₁₋₄ alkyl. An alkoxy may be substituted or unsubstituted.

As used herein, “acyl” refers to a hydrogen an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

As used herein, “alkoxyalkyl” refers to an alkyl group in which one or more of the hydrogen atoms are replaced by an alkoxy group. Exemplary alkoxyalkyl groups include but are not limited to, methoxymethyl, ethoxymethyl, methoxyethyl and ethoxyethyl. An alkoxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to a O-alkyl group and O-monocyclic cycloalkyl group in which one or more of the hydrogen atoms are replaced by a halogen (e.g., mono-haloalkoxy, di-haloalkoxy and tri-haloalkoxy). In some instances, a haloalkoxy can be —OR, wherein R is a C₁₋₄ alkyl substituted by 1, 2 or 3 halogens. Such groups include but are not limited to, chloromethoxy, fluoromethoxy, difluoromethoxy, trifluoro-2-ethoxy, trifluoromethoxy, 1-chloro-2-fluoromethoxy, 2-fluoroisobutoxy, chloro-substituted cyclopropyl, fluoro-substituted cyclopropyl, chloro-substituted cyclobutyl and fluoro-substituted cyclobutyl. A haloalkoxy may be substituted or unsubstituted.

A “sulfenyl” group refers to an “—SR” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} is hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—SCN” group.

An “isothiocyanato” group refers to an “—NCS” group.

A “mercapto” group refers to an “—SH” group.

A “carbonyl” group refers to a —C(═O)— group.

An “S-sulfonamido” group refers to a “—SO₂N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-amido may be substituted or unsubstituted.

A “mono-substituted amine” refers to a “—NHR_{A}” in which R_{A} can be independently alkyl, alkenyl, alkynyl, cycloalkyl, cycloalkenyl, aryl, heteroaryl, heterocyclyl, aryl(alkyl), heteroaryl(alkyl) or heterocyclyl(alkyl). A mono-substituted amine may be substituted or unsubstituted. In some instances, a mono-substituted amine can be —NHR_{A}, wherein R_{A} can be an unsubstituted C₁₋₆ alkyl or an unsubstituted or a substituted benzyl.

A “di-substituted amine” refers to a “—NR_{A}R_{B}” in which R_{A} and R_{B} can be independently can be independently alkyl, alkenyl, alkynyl, cycloalkyl, cycloalkenyl, aryl, heteroaryl, heterocyclyl, aryl(alkyl), heteroaryl(alkyl) or heterocyclyl(alkyl). A mono-substituted amine may be substituted or unsubstituted. In some instances, a mono-substituted amine can be —NR_{A}R_{B}, wherein R_{A} and R_{B} can be independently an unsubstituted C₁₋₆ alkyl or an unsubstituted or a substituted benzyl.

The term “halogen atom” or “halogen” as used herein, means any one of the radio-stable atoms of column 7 of the Periodic Table of the Elements, such as, fluorine, chlorine, bromine and iodine.

The term “—O-linked α-amino acid” refers to an α-amino acid that is attached to the indicated moiety via the hydroxy from its main-chain carboxylic acid group. When the amino acid is attached in an —O-linked amino acid, the hydrogen that is part of the hydroxy from its main-chain carboxylic acid group is not present and the amino acid is attached via the oxygen. O-linked amino acids can be substituted or unsubstituted.

Where the numbers of substituents are not specified (e.g., haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

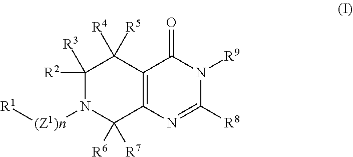

wherein: n can be 0 or 1; Z¹ can be —C(═O)— or —NH—C(═O)—; R¹ can be selected from an optionally substituted aryl, an optionally substituted heteroaryl, an optionally substituted heterocyclyl, an optionally substituted aryl(C₁₋₄ alkyl), an optionally substituted heteroaryl(C₁₋₄ alkyl) and an optionally substituted heterocyclyl(C₁₋₄ alkyl); R² and R³ can be independently hydrogen or an unsubstituted C₁₋₄ alkyl; R⁴, R⁵, R⁶ and R⁷ can be each hydrogen; R⁸ can be —NR^{10A}R^{10B} or an optionally substituted C₂₋₁₂ alkynyl, wherein the C₂₋₁₂ alkynyl can be optionally substituted with one or more substituents selected from amino, —NH—C(═O)(an unsubstituted C₁₋₄ alkyl), hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl an unsubstituted 4-6 membered monocyclic heterocyclyl, an optionally substituted aryl and an optionally substituted 5-6 membered monocyclic heteroaryl; R⁹ can be a substituted phenyl, a substituted monocyclic heteroaryl or a substituted fused-bicyclic heteroaryl, wherein the substituted phenyl, the substituted monocyclic heteroaryl or the substituted fused-bicyclic heteroaryl is substituted with —C(═O)NR¹¹R¹² and the substituted phenyl, the substituted monocyclic heteroaryl or the substituted fused-bicyclic heteroaryl can be optionally further substituted; R^{10A} can be hydrogen, an unsubstituted C₁₋₆ alkyl, a monocyclic C₃₋₆ cycloalkyl optionally substituted with one or two halogens, an optionally substituted 5-6 membered monocyclic heteroaryl, an optionally substituted 4-6 membered monocyclic heterocyclyl or an optionally substituted monocyclic C₃₋₆ cycloalkyl(C₁₋₄ alkyl); R^{10B} can be selected from an optionally substituted C₂₋₈ alkenyl, an optionally substituted C₂₋₈ alkynyl, an optionally substituted aryl, an optionally substituted aryl(C₁₋₄ alkyl), an optionally substituted heteroaryl(C₁₋₄ alkyl) and an optionally substituted heterocyclyl(C₁₋₄ alkyl), wherein the C₂₋₈ alkenyl and the C₂₋₈ alkynyl is optionally substituted with one or more substituents selected from the group consisting of amino, hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl and an unsubstituted 4-6 membered monocyclic heterocyclyl; R¹¹ can be H (hydrogen) or an unsubstituted C₁₋₄ alkyl; R¹² can be —(C(═O)—O—CR^{13A}R^{13B})p-R¹⁴; p can be 0 or 1; R^{13A} and R^{13B} can be independently H (hydrogen) or an unsubstituted C₁₋₄ alkyl; and R¹⁴ can be selected from

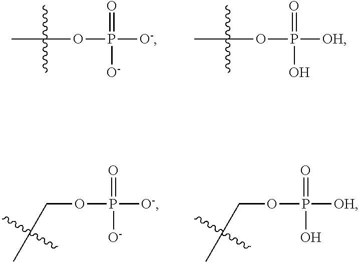

—O-linked α-amino acid, —CH₂—O-linked α-amino acid, a hydroxy-substituted C₁₋₄ alkyl, —O—(C═O)—CH(OH)—CH(OH)—C(═O)OH, —O—(C═O)—CH₂—CH(OH)—C(═O)OH, —O—(C═O)—CH═CH—C(═O)OH and —(C═O)-(an unsubstituted or a substituted monocyclic heterocyclyl).

As provided herein, various groups can be attached to the piperidinyl ring of the ring structure of Formula (I). In some embodiments, n can be 1; Z¹ can be —C(═O)—; and R¹ can be selected from an optionally substituted aryl, an optionally substituted heteroaryl, an optionally substituted heterocyclyl, an optionally substituted aryl(C₁₋₄ alkyl), an optionally substituted heteroaryl(C₁₋₄ alkyl) and an optionally substituted heterocyclyl(C₁₋₄ alkyl). In other embodiments, n can be 1; Z¹ can be —NH—C(═O)—; and R¹ can be selected from an optionally substituted aryl, an optionally substituted heteroaryl, an optionally substituted heterocyclyl, an optionally substituted aryl(C₁₋₄ alkyl), an optionally substituted heteroaryl(C₁₋₄ alkyl) and an optionally substituted heterocyclyl(C₁₋₄ alkyl). As shown below, when Z¹ is —C(═O)— or —NH—C(═O)—, Formula (I) can be Formula (Ia) or (Ib), or a pharmaceutically acceptable salt thereof, respectively.

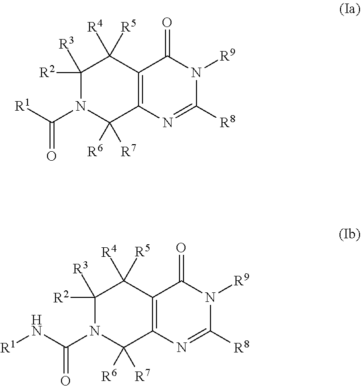

Various cyclic moieties can be present for R¹. In some embodiments, R¹ can be a carbocyclic moiety, for example an optionally substituted aryl. For example, R¹ can be an optionally substituted phenyl. In some embodiments, R¹ can be an unsubstituted phenyl. In other embodiments, R¹ can be a substituted phenyl. When R¹ is a substituted phenyl, the phenyl can be mono-substituted. The mono-substituted phenyl can be a para-substituted phenyl, a meta-substituted phenyl or an ortho-substituted phenyl. The substituted phenyl can be substituted by multiple moieties, such as 2, 3 or more than 3 times. For example, the substituted phenyl of R¹ can be di-substituted (such as a meta- and para-substituted phenyl). When more than one moiety is present, the moieties can be the same or different moieties.

As described herein, R¹ can be a cyclic moiety, including a cyclic moiety that can include one or more heteroatoms in the ring(s). In some embodiments, R¹ can be an optionally substituted heteroaryl. The heteroaryl can be monocyclic or bicyclic. In some embodiments, R¹ can be an unsubstituted or a substituted monocyclic heteroaryl. For example, R¹ can be a 5-membered or 6-membered monocyclic heteroaryl. In other embodiments, R¹ can be an unsubstituted or a substituted bicyclic heteroaryl. The bicyclic heteroaryl can be a 9-membered or 10-membered heteroaryl. The heteroaryl can include one or more heteroatoms (such as 1, 2 or 3), such as N (nitrogen), O (oxygen) and/or S (sulfur). In some embodiments, R¹ can be an optionally substituted heterocyclyl. The heterocyclyl can be a monocyclic heterocyclyl or a bicyclic heterocyclyl. In some embodiments, R¹ can be an unsubstituted or a substituted monocyclic heterocyclyl, such as a 5-membered or 6-membered monocyclic heterocyclyl. In other embodiments, R¹ can be an unsubstituted or a substituted bicyclic heterocyclyl, including a 9-membered or 10-membered heterocyclyl. The number and types of heteroatoms that can be present in a heterocyclyl can vary. As an example, 1, 2, 3 or more than 3 heteroatoms, such as N (nitrogen), O (oxygen) and/or S (sulfur), can be present in a heterocyclyl of R¹.

In some embodiments, R¹ can be selected from an unsubstituted or a substituted [5,5] bicyclic heteroaryl, an unsubstituted or a substituted [5,6] bicyclic heteroaryl, an unsubstituted or a substituted [6,5] bicyclic heteroaryl, an unsubstituted or a substituted [6,6] bicyclic heteroaryl, an unsubstituted or a substituted [5,5] bicyclic heterocyclyl, an unsubstituted or a substituted [5,6] bicyclic heterocyclyl, an unsubstituted or a substituted [6,5] bicyclic heterocyclyl and an unsubstituted or a substituted [6,6] bicyclic heterocyclyl. In some embodiments, R¹ can be a nitrogen-containing, bicyclic heteroaryl. In other embodiments, R¹ can be a nitrogen-containing, bicyclic heterocyclyl. In some embodiments, R¹ can have the general structure

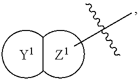

wherein Ring Z¹ indicates the point of attachment to the remaining portion of Formula (I); and wherein Ring Y¹ and Ring Z¹ can be independently selected from phenyl, furan, furazan, thiophene, phthalazine, pyrrole, oxazole, 1,2,3-oxadiazole, 1,2,4-oxadiazole, thiazole, 1,2,3-thiadiazole, 1,2,4-thiadiazole, imidazole, pyrazole, isoxazole, isothiazole, triazole, thiadiazole, tetrazole, pyridine, pyridazine, pyrimidine, pyrazine, 1,2,3-triazine, 1,2,4-triazine, 1,2,3,4-tetrazine, 2H-1,2-oxazine, hexahydro-1,3,5-triazine, imidazoline, imidazolidine, isoxazoline, isoxazolidine, oxazoline, oxazolidine, thiazoline, thiazolidine, morpholine, piperidine, piperazine, pyrrolidine, pyrazoline, pyrazolidine and thiamorpholine, wherein Ring Y¹ and Ring Z¹ can be each optionally substituted. In some embodiments, Ring Y¹ can be selected from an optionally substituted phenyl, an optionally substituted pyridine, an optionally substituted pyridazine, an optionally substituted pyrimidine, an optionally substituted pyrazine, an optionally substituted 1,2,3-triazine, an optionally substituted 1,2,4-triazine and an optionally substituted 1,2,3,4-tetrazine. In some embodiments, Ring Z¹ can be selected from an optionally substituted phenyl, an optionally substituted pyridine, an optionally substituted pyridazine, an optionally substituted pyrimidine, an optionally substituted pyrazine, an optionally substituted 1,2,3-triazine, an optionally substituted 1,2,4-triazine and an optionally substituted 1,2,3,4-tetrazine. In other embodiments, Ring Z¹ can be selected from an optionally substituted furan, an optionally substituted thiophene, an optionally substituted pyrrole, an optionally substituted oxazole, an optionally substituted thiazole, an optionally substituted imidazole, an optionally substituted pyrazole, an optionally substituted isoxazole and an optionally substituted isothiazole.

Various cyclic groups can be attached via a C₁₋₄ alkyl linker for R¹. In some embodiments, R¹ can be an optionally substituted aryl(C₁₋₄ alkyl). An exemplary optionally substituted aryl(C₁₋₄ alkyl) is an optionally substituted benzyl. In other embodiments, R¹ can be an optionally substituted heteroaryl(C₁₋₄ alkyl). In still other embodiments, R¹ can be an optionally substituted heterocyclyl(C₁₋₄ alkyl). Examples of heteroaryls and heterocyclyls are described herein and include those of the previous paragraph. As described herein, the linker can include 1 to 4 carbons. In some embodiments, the C₁₋₄ alkyl linker for R¹ can be —CH₂—, —CH₂CH₂—, —CH₂CH₂CH₂— or —CH₂CH₂CH₂CH₂—. Further as described herein lower alkylene linker (C₁₋₄ alkyl linker) for R¹ can be substituted. Examples of substituents that can be present on a substituted lower alkylene linker (C₁₋₄ alkyl linker) for aryl(C₁₋₄ alkyl), heteroaryl(C₁₋₄ alkyl) and heterocyclyl(C₁₋₄ alkyl) include an unsubstituted C₁₋₄ haloalkyl (such as CF₃).

As described herein, R¹ can be substituted. A variety of substituents can substitute the R¹ groups described herein. In some embodiments, R¹ can be substituted with one or more substituents (for example, 1, 2 or 3) independently selected from deuterium, halogen (such as F, Cl and/or Br), cyano, an unsubstituted C₁₋₆ alkyl (for example, methyl, ethyl, n-propyl, isopropyl, n-butyl, iso-butyl, sec-butyl, tert-butyl, pentyl (straight-changed or branched) and hexyl (straight-chained or branched)), an unsubstituted C₂₋₆ alkenyl (for example, ethenyl, propenyl and butenyl), an unsubstituted C₂₋₆ alkynyl (for example, ethynyl, propynyl and butynyl), an unsubstituted or a substituted monocyclic C₃₋₆ cycloalkyl (such as cyclopropyl, cyclobutyl, cyclopentyl and cyclohexyl, including halo-substituted versions of each of the aforementioned examples), an unsubstituted C₁₋₆ haloalkyl (such as —CHF₂, —CH₂F, —CF₃, —CHClF, —CH₂Cl, —CHCl₂, —CCl₃, —CH₂CHF₂, —CH₂CH₂F, —CH₂CF₃, —CH₂CHClF,

—CH₂CH₂Cl, —CH₂CHCl₂, —CH₂CCl₃, —CH(CH₃)CF₃, —CH(CH₃)CHF₂, —C(CH₃)₂CF₃ and

—C(CH₃)₂CHF₂), an unsubstituted C₁₋₆ alkoxy (for example, methoxy, ethoxy, isopropoxy, n-butoxy, sec-butoxy, isobutoxy, tert-butoxy, —O-(cyclopropyl), —O-(cyclobutyl) and —O-(oxetane)), an unsubstituted C₁₋₆ haloalkoxy (for example, —OCHF₂, —OCH₂F, —OCF₃,

—OCHClF, —OCH₂Cl, —OCHCl₂, —OCCl₃, —OCH₂CHF₂, —OCH₂CH₂F, —OCH₂CF₃,

—OCH₂CHClF, —OCH₂CH₂Cl, —OCH₂CHCl₂, —OCH₂CCl₃, —OCH(CH₃)CF₃,

—OCH(CH₃)CHF₂, —OC(CH₃)₂CF₃, —OC(CH₃)₂CHF₂, —O(halo-substituted cyclopropyl) and

—O(halo-substituted cyclobutyl)), an unsubstituted acyl (for example, —C(═O)—C₁₋₄ alkyl), an unsubstituted C-amido (such as —C(═O)NH₂ and —C(═O)NH—C₁₋₄ alkyl), an unsubstituted sulfonyl (such as —S(═O)₂—C₁₋₄ alkyl), an unsubstituted amino, a mono-substituted amine (for example, an mono-alkyl substituted amine) and a di-substituted amine (such as a di-alkyl substituted amine). In some embodiments, R¹ can be substituted with one or more substituents (such as 1, 2 or 3) independently selected from halogen (such as F, Cl and/or Br), cyano, an unsubstituted C₁₋₆ alkyl (such as methyl) and an unsubstituted C₂₋₆ alkynyl (for example, ethynyl).

The number of substituents present on a substituted R¹ group can vary. In some embodiments, R¹ is substituted with 1 substituent. In other embodiments, R¹ is substituted with 2 substituents. In still other embodiments, R¹ is substituted with 3 substituents.

Exemplary R¹ groups include, but are not limited to, the following:

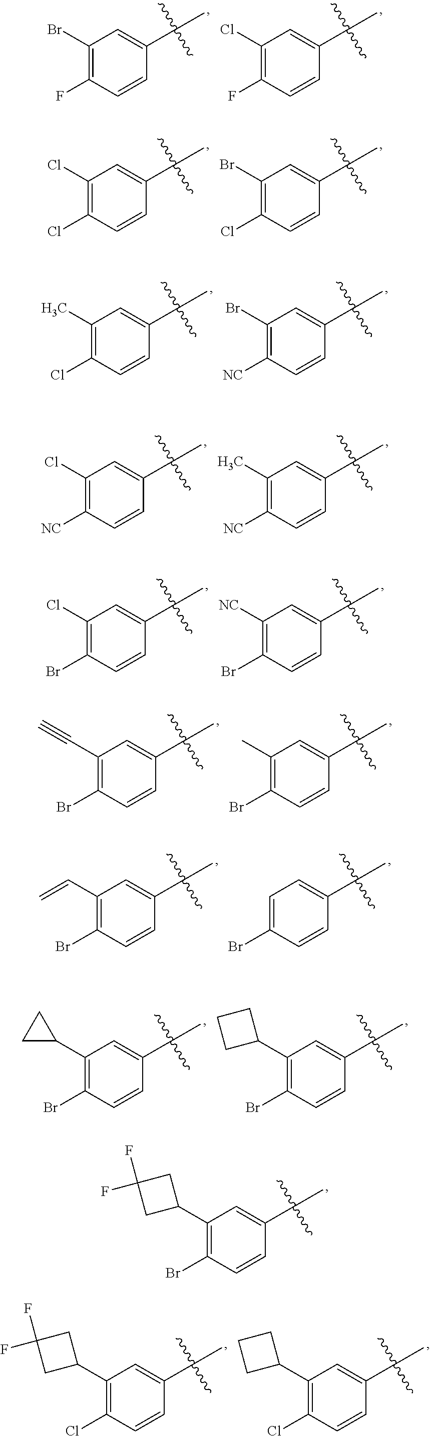

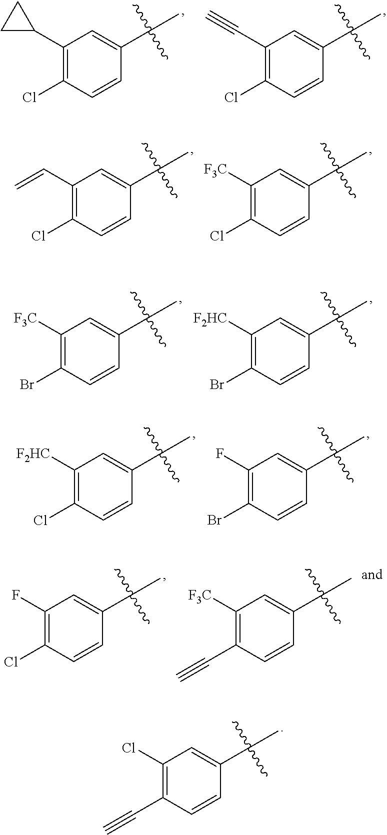

In addition to the groups described herein that can be attached to the piperazine ring of the tetracyclic ring structure of Formula (I), the piperidinyl ring can be further unsubstituted or substituted. In some embodiments, R² can be hydrogen. In other embodiments, R² can be an unsubstituted C₁₋₄ alkyl. For example, R² can be methyl, ethyl, n-propyl, iso-propyl, n-butyl, iso-butyl, sec-butyl and tert-butyl.

In some embodiments, R³ can be hydrogen. In other embodiments, R³ can be an unsubstituted C₁₋₄ alkyl, such as methyl, ethyl, n-propyl, iso-propyl, n-butyl, iso-butyl, sec-butyl and tert-butyl. In some embodiments, R² can be hydrogen; and R³ can be an unsubstituted C₁₋₄ alkyl, for example, those unsubstituted C₁₋₄ alkyls described herein. In some embodiments, R², R³, R⁴, R⁵, R⁶ and R⁷ can be each hydrogen. When one R² is hydrogen and R³ is an unsubstituted C₁₋₄ alkyl, a stereocenter may be formed. In some embodiments, the stereocenter that is formed can be in the (R)-configuration. In other embodiments, the stereocenter that is formed can be in the (S)-configuration.

An R¹ substituent can be an amine, such as an amine having the general formula —NR^{10A}R^{10B}. In some embodiments, R^{10A} can be hydrogen. In other embodiments, R^{10A} can be an unsubstituted C₁₋₆ alkyl, such as methyl, ethyl, n-propyl, iso-propyl, n-butyl, iso-butyl, sec-butyl, tert-butyl, pentyl (straight-chained and branched) and hexyl (straight-chained and branched). In still other embodiments, R^{10A} can be a monocyclic C₃₋₆ cycloalkyl optionally substituted with one or two halogens. For example, R^{10A} can be a monocyclic C₃₋₆ cycloalkyl that is substituted with 1 or 2 fluoros. In some embodiments, R^{10A} can be an unsubstituted C₃₋₆ cycloalkyl. In other embodiments, R^{10A} can be an optionally substituted 5-6-membered monocyclic heteroaryl. In still other embodiments, R^{10A} can be an optionally substituted 4-6 membered monocyclic heterocyclyl. As an example, R^{10A} can be an optionally substituted 4-6 membered monocyclic heterocyclyl that includes 1-3 heteroatoms selected from O (oxygen), S (sulfur) and N (nitrogen). In some embodiments, R^{10A} can be azetidine, oxetane or thietane, wherein each of the aforementioned can be unsubstituted or substituted. Examples of groups that can be present on a substituted 4-6 membered monocyclic heterocyclyl are fluoro, chloro, methyl, ethyl and an unsubstituted C₁₋₄ haloalkyl (such as CF₃, CHF₂ and CH₂F). In other embodiments, R^{10A} can be an optionally substituted monocyclic C₃₋₆ cycloalkyl(C₁₋₄ alkyl). Exemplary monocyclic C₃₋₆ cycloalkyl(C₁₋₄ alkyl) moieties include, but are not limited to, cyclopropyl-CH₂—, cyclobutyl-CH₂—, cyclopentyl-CH₂—, cyclohexyl-CH₂—, cyclopropyl-CH₂CH₂—, cyclobutyl-CH₂CH₂—, cyclopentyl-CH₂CH₂— and cyclohexyl-CH₂CH₂—.

For R^{10B}, R^{10B} can be an optionally substituted C₂₋₈ alkenyl, an optionally substituted C₂₋₈ alkynyl, an optionally substituted aryl, an optionally substituted aryl(C₁₋₄ alkyl), an optionally substituted heteroaryl(C₁₋₄ alkyl) and an optionally substituted heterocyclyl(C₁₋₄ alkyl). In some embodiments, R^{10B} can be an unsubstituted C₂₋₈ alkenyl. In other embodiments, R^{10B} can be a C₂₋₈ alkenyl substituted with amino, hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl and/or an unsubstituted 4-6 membered monocyclic heterocyclyl. In still other embodiments, R^{10B} can be an unsubstituted C₂₋₈ alkynyl. In yet still other embodiments, R^{10B} can be a substituted C₂₋₈ alkynyl substituted with amino, hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl and/or an unsubstituted 4-6 membered monocyclic heterocyclyl.

A variety of groups can be present on a substituted C₂₋₈ alkenyl and a substituted C₂₋₈ alkynyl. In some embodiments, the substituted C₂₋₈ alkenyl and/or the substituted C₂₋₈ alkynyl can be substituted with amino, hydroxy and/or an unsubstituted C₁₋₄ alkoxy. Examples of unsubstituted C₁₋₄ alkoxys include methoxy, ethoxy, n-propoxy, iso-propoxy, n-butoxy, iso-butoxy, sec-butoxy and tert-butoxy. In some embodiments, the substituted C₂₋₈ alkenyl and/or the substituted C₂₋₈ alkynyl can be substituted with an unsubstituted C₁₋₄ haloalkyl, such as —CF₃, —CHF₂, —CH₂F, —CCl₃, —CHCl₂ and —CH₂Cl.

When the C₂₋₈ alkenyl and/or the C₂₋₈ alkynyl is substituted with an unsubstituted C₃₋₄ monocyclic cycloalkyl, a fluoro-substituted C₃₋₄ monocyclic cycloalkyl, a hydroxy-substituted C₃₋₄ monocyclic cycloalkyl and/or an unsubstituted 4-6 membered monocyclic heterocyclyl, the unsubstituted C₃₋₄ monocyclic cycloalkyl, the fluoro-substituted C₃₋₄ monocyclic cycloalkyl, the hydroxy-substituted C₃₋₄ monocyclic cycloalkyl and/or the unsubstituted 4-6 membered monocyclic heterocyclyl can replace a single hydrogen of the C₂₋₈ alkenyl and/or the C₂₋₈ alkynyl. In other instances, the unsubstituted C₃₋₄ monocyclic cycloalkyl, the fluoro-substituted C₃₋₄ monocyclic cycloalkyl, the hydroxy-substituted C₃₋₄ monocyclic cycloalkyl and/or the unsubstituted 4-6 membered monocyclic heterocyclyl can replace two hydrogens on the same carbon of the C₂₋₈ alkenyl and/or the C₂₋₈ alkynyl such that the unsubstituted C₃₋₄ monocyclic cycloalkyl, the fluoro-substituted C₃₋₄ monocyclic cycloalkyl, the hydroxy-substituted C₃₋₄ monocyclic cycloalkyl and/or the unsubstituted 4-6 membered monocyclic heterocyclyl is connected to the C₂₋₈ alkenyl and/or the C₂₋₈ alkynyl in a spiro-fashion. In some embodiments, the C₃₋₄ monocyclic cycloalkyl substituted on the C₂₋₈ alkenyl and/or the C₂₋₈ alkynyl can be cyclopropyl. In other embodiments, the C₃₋₄ monocyclic cycloalkyl substituted on the C₂₋₈ alkenyl and/or the C₂₋₈ alkynyl can be cyclobutyl. In some embodiments, the unsubstituted 4-6 membered monocyclic heterocyclyl substituted on the C₂₋₈ alkenyl and/or the C₂₋₈ alkynyl can be azetidine, oxetane or thietane.

Examples of R^{10B} being an optionally substituted C₂₋₈ alkenyl or an optionally substituted C₂₋₈ alkynyl include:

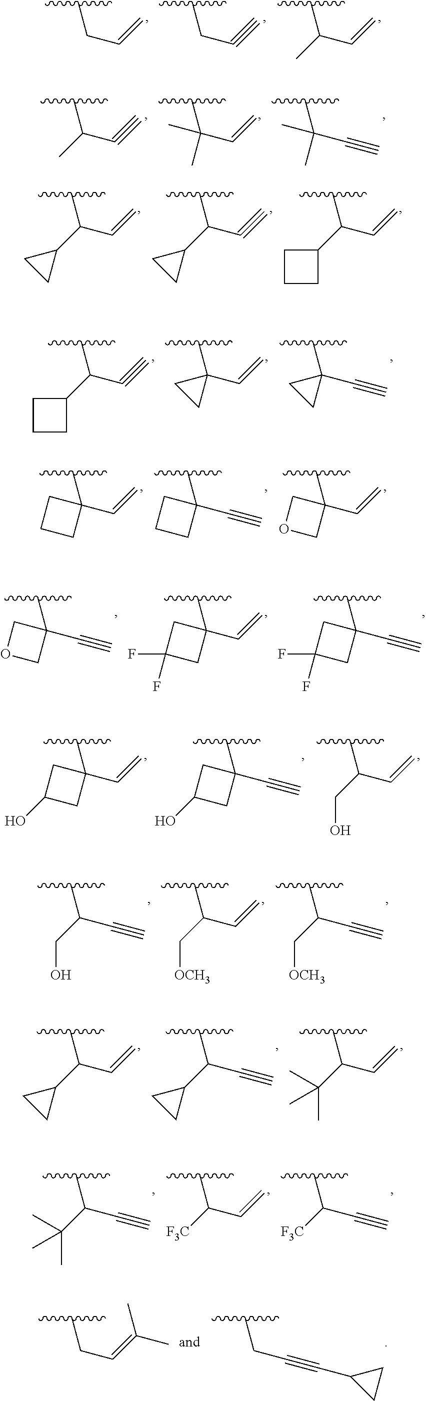

As provided herein, R can include a cyclic moiety. an optionally substituted aryl, an optionally substituted aryl(C₁₋₄ alkyl), an optionally substituted heteroaryl(C₁₋₄ alkyl) and an optionally substituted heterocyclyl(C₁₋₄ alkyl). In some embodiments, R^{10B} can be an optionally substituted aryl, such as an optionally substituted phenyl or an optionally substituted naphthyl. In still other embodiments, R^{10B} can be an optionally substituted aryl(C₁₋₄ alkyl), such as an optionally substituted benzyl. In yet still other embodiments, R^{10B} can be an optionally substituted heteroaryl(C₁₋₄ alkyl). In some embodiments, R^{10B} can be an optionally substituted heterocyclyl(C₁₋₄ alkyl). The heteroaryl and heterocyclyl that can be attached via a C₁₋₄ alkyl linker for R^{10B} can be monocyclic or bicyclic. Examples of a heteroaryl that can be attached via C₁₋₄ alkyl linker are a 5- to 6-membered monocyclic heteroaryl that includes 1-5 heteroatoms selected from N (nitrogen), O (oxygen) and S (sulfur) and a 9- to 10-membered bicyclic heteroaryl that includes 1-5 heteroatoms selected from N (nitrogen), O (oxygen) and S (sulfur). When R^{10B} can be an optionally substituted heterocyclyl(C₁₋₄ alkyl), in some embodiments, R^{10B} can be an optionally substituted monocyclic 5- to 6-membered heterocyclyl or an optionally substituted bicyclic 9- to 10-membered heterocyclyl, wherein the heterocyclyl can include 1 or more heteroatoms selected from O (oxygen), S (sulfur) and N (nitrogen). The bicyclic heteroaryls and bicyclic heterocyclyls for R^{10B} can be fused wherein the rings are connected via two adjacent ring atoms or spiro-cyclic wherein the rings are connected via 1 ring atom.

As described herein, the C₁₋₄ alkyl of the optionally substituted aryl(C₁₋₄ alkyl), the optionally substituted heteroaryl(C₁₋₄ alkyl) and the optionally substituted heterocyclyl(C₁₋₄ alkyl) for R^{10B} can be optionally substituted with an unsubstituted C₁₋₃ alkyl (for example, methyl, ethyl, n-propyl and isopropyl) or an unsubstituted C₃₋₄ monocyclic cycloalkyl (such as cyclopropyl and cyclobutyl). Further, the aryl of an aryl(C₁₋₄ alkyl), the heteroaryl of a heteroaryl(C₁₋₄ alkyl) and the heterocyclyl of a heterocyclyl(C₁₋₄ alkyl) can be unsubstituted or substituted. For example, the aryl of an aryl(C₁₋₄ alkyl), the heteroaryl of a heteroaryl(C₁₋₄ alkyl) and the heterocyclyl of a heterocyclyl(C₁₋₄ alkyl) can be substituted with one or more moieties (for example, 1, 2 or 3) selected from halogen, an unsubstituted C₂₋₅ alkenyl, a substituted C₂₋ₛ alkenyl, an unsubstituted C₂₋₅ alkynyl, a substituted C₂₋₅ alkynyl, an unsubstituted monocyclic heteroaryl (for example, a 5- to 6-membered monocyclic heteroaryl containing 1-3 heteroatoms selected from O (oxygen), S (sulfur) and N (nitrogen)) and a substituted monocyclic heteroaryl (for example, a 5- to 6-membered monocyclic heteroaryl containing 1-3 heteroatoms selected from O (oxygen), S (sulfur) and N (nitrogen)).

In some embodiments, R⁸ can be —NR^{10A}R^{10B}, R^{10A} can be hydrogen; and R^{10B} can be an unsubstituted C₂₋₈ alkenyl. In other embodiments, R⁸ can be —NR^{10A}R^{10B}, R^{10A} can be hydrogen; and R^{10B} can be a substituted C₂₋₈ alkenyl. In still other embodiments, R⁸ can be —NR^{10A}R^{10B}, R^{10A} can be hydrogen; and R^{10B} can be an unsubstituted C₂₋₈ alkynyl. In yet still other embodiments, R⁸ can be —NR^{10A}R^{10B}, R^{10A} can be hydrogen; and R^{10B} can be a substituted C₂₋₈ alkynyl. A variety of suitable C₂₋₈ alkenyls and C₂₋₈ alkynyl are described herein.

Various cyclic moieties can be present for R^{10A} and R^{10B}. In some embodiments, R⁸ can be —NR^{10A}R^{10B}; R^{10A} can be a monocyclic C₃₋₆ cycloalkyl optionally substituted with one or two halogens or an optionally substituted 4-6 membered monocyclic heterocyclyl; and R^{10B} can be an unsubstituted aryl(C₁₋₄ alkyl) or a substituted aryl(C₁₋₄ alkyl), such as an unsubstituted benzyl or a substituted benzyl. In other embodiments, R⁸ can be —NR^{10A}R^{10B}; R^{10A} can be an optionally substituted 5-6 membered monocyclic heteroaryl; and R^{10B} can be an unsubstituted aryl(C₁₋₄ alkyl) or a substituted aryl(C₁₋₄ alkyl), such as an unsubstituted benzyl or a substituted benzyl. In other embodiments, R⁸ can be —NR^{10A}R^{10B}; R^{10A} can be an optionally substituted monocyclic C₃₋₆ cycloalkyl(C₁₋₄ alkyl); and R^{10B} can be an unsubstituted aryl(C₁₋₄ alkyl) or a substituted aryl(C₁₋₄ alkyl). For example, R^{10A} can be an optionally substituted cyclopropyl-CH₂—, an optionally substituted cyclobutyl-CH₂—, an optionally substituted cyclopentyl-CH₂— or an optionally substituted cyclohexyl-CH₂—; and R^{10B} can be an unsubstituted benzyl or a substituted benzyl. In some embodiments, R⁸ can be —NR^{10A}R^{10B}; R^{10A} can be an optionally substituted monocyclic C₃₋₆ cycloalkyl(C₁₋₄ alkyl); and R^{10B} can be a substituted aryl. As an example, R^{10A} can be an optionally substituted cyclopropyl-CH₂—, an optionally substituted cyclobutyl-CH₂—, an optionally substituted cyclopentyl-CH₂— or an optionally substituted cyclohexyl-CH₂—; and R^{10B} can be an optionally substituted phenyl.

In some embodiments, when R^{10A} is an optionally substituted monocyclic C₃₋₆ cycloalkyl(C₁₋₄ alkyl), then R^{10B} cannot be an unsubstituted C₂₋₈ alkenyl. In some embodiments, R^{10A} cannot be an optionally substituted monocyclic C₃₋₆ cycloalkyl(C₁₋₄ alkyl), such as unsubstituted cyclopropyl-CH₂— or substituted cyclopropyl-CH₂—.

In some embodiments, R⁸ can be an unsubstituted C₂₋₁₂ alkynyl. In other embodiments, R⁸ can be a substituted C₂₋₁₂ alkynyl substituted with one or more substituents (such as 1, 2 or 3) selected from amino, —NH—C(═O)(an unsubstituted C₁₋₄ alkyl), hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl, an unsubstituted 4-6 membered monocyclic heterocyclyl, an optionally substituted aryl and an optionally substituted 5-6 membered monocyclic heteroaryl. In some embodiments, R⁸ as C₂₋₁₂ alkynyl can be substituted with an unsubstituted or a substituted phenyl. In other embodiments, R⁸ as C₂₋₁₂ alkynyl can be substituted with an unsubstituted or a substituted 5-6 membered monocyclic heteroaryl. For example, R⁸ can be C₂₋₁₂ alkynyl substituted with a 5-6 membered monocyclic heteroaryl that includes 1, 2 or 3 heteroatoms independently selected from N (nitrogen), O (oxygen) and S (sulfur). In other embodiments, R⁸ can be C₂₋₁₂ alkynyl substituted with an unsubstituted 4-6 membered monocyclic heterocyclyl, such as an unsubstituted 4-6 membered monocyclic heterocyclyl that includes 1, 2 or 3 heteroatoms independently selected from N (nitrogen), O (oxygen) and S (sulfur). In some embodiments, when R⁸ is a C₂₋₁₂ alkynyl substituted with substituted aryl or a substituted 5-6 membered monocyclic heteroaryl, then the aryl and/or the heteroaryl can be substituted one or more times (1, 2, 3 or 4 times) with a moiety independently selected from halogen (such as F, Cl or Br) and an unsubstituted C₁₋₃ alkyl (for example, methyl, ethyl, n-propyl and isopropyl). Exemplary R⁸ moieties include, but are not limited to,

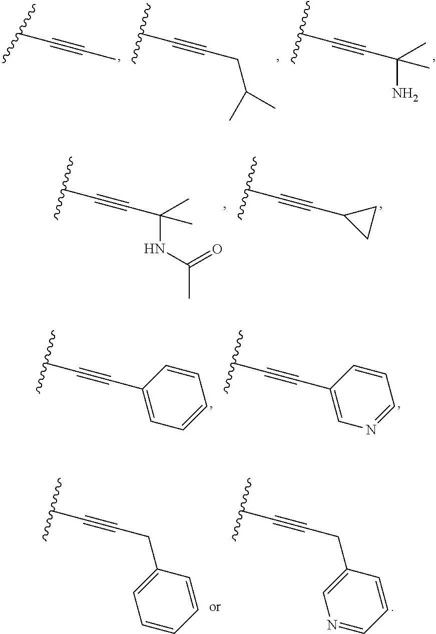

As provided herein, R⁹ can be a cyclic moiety that is substituted with

—C(═O)NR¹¹R¹². For example, in some embodiments, R⁹ can be a substituted phenyl. In other embodiments, R⁹ can be a heteroaryl (monocyclic or fused-bicyclic heteroaryl). The heteroaryl can have one or more heteroatoms present, for example, 1, 2 or 3 heteroatoms. Exemplary heteroatoms include, but are not limited to, N (nitrogen), O (oxygen) and S (sulfur). The size of the heteroaryl can vary. In some embodiments, R⁹ can be a substituted monocyclic heteroaryl. The monocyclic heteroaryl can be a 5- or 6-membered heteroaryl. In other embodiments, R⁹ can be a substituted fused-bicyclic heteroaryl. The number of ring atoms of the fused-bicyclic heteroaryl can be 9 or 10 such that R⁹ can be a substituted fused-bicyclic 9- or 10-membered heteroaryl. Examples of suitable heteroaryls for R⁹ include pyrazole, imidazole, pyridine, pyrimidine, pyrazine, pyridazine, indazole, benzo[d]imidazole and imidazo[4,5-b]pyridine. In some embodiments, R⁹ can be selected from:

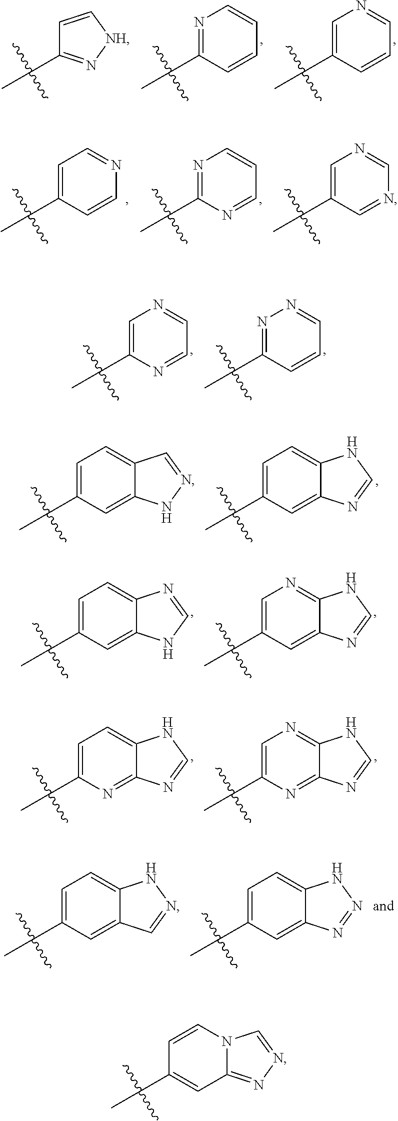

wherein each of the aforementioned can be unsubstituted or substituted as described herein. In some embodiment, R⁹ can be

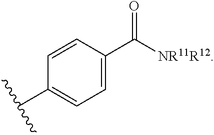

As provided herein, R⁹ is substituted with —C(═O)NR¹¹R¹², and can be optionally further substituted. Additionally substituents that can be present on R⁹ in addition to —C(═O)NR¹¹R¹² include, but are not limited to, halogen (such as F, Cl or Br), an unsubstituted C₁₋₄ alkyl, a cyano-substituted C₁₋₄ alkyl, an unsubstituted C₁₋₄ haloalkyl, an unsubstituted C₁₋₄ alkoxy, a hydroxy-substituted C₁₋₄ alkoxy, an optionally substituted monocyclic C₃₋₆ cycloalkyl (such as cyclopropyl, cyclobutyl, cyclopentyl and cyclohexyl), an optionally substituted monocyclic heteroaryl, an optionally substituted monocyclic heterocyclyl, amino, a mono-substituted amine and a di-substituted amine. Examples of an unsubstituted C₁₋₄ alkyl and an unsubstituted C₁₋₄ alkoxy include the following: methyl, ethyl, n-propyl, isopropyl, n-butyl, sec-butyl, iso-butyl, tert-butyl, methoxy, ethoxy, n-propoxy, isopropoxy, n-butoxy, sec-butoxy, iso-butoxy and tert-butoxy. Exemplary unsubstituted C₁₋₄ haloalkyls include —CHF₂, —CH₂F, —CF₃, —CH₂Cl, —CHCl₂, —CCl₃, —CH₂CF₃, —CH₂CHF₂, —CH₂CCl₃ and —CH₂CHCl₂. A non-limiting list of cyano-substituted C₁₋₄ alkyls include —CH₂CN, —CH₂CH₂CN, —CH₂CH(CH₃)CN and —CH₂C(CH₃)₂CN. Examples of hydroxy-substituted C₁₋₄ alkoxy include —OCH₂CH₂OH, —OCH₂CH(CH₃)OH and —OCH₂C(CH₃)₂OH. The number of additional substituents that can be present on R⁹ can vary. In some embodiments, 1 additional substituent can be present on R⁹. In some embodiments, 2 additional substituents can be present on R⁹.

An R⁹ group described herein, can be substituted with an unsubstituted or a substituted monocyclic heteroaryl, such as a 5- or 6-membered monocyclic heteroaryl that includes one or more heteroatoms (such as 1, 2 or 3) selected from O (oxygen), S (sulfur) and N (nitrogen). Suitable monocyclic heteroaryls that can be present on R⁹ are described herein, and include pyrazole, imidazole, pyridine, pyrimidine, pyrazine, pyridazine, pyridazine, pyridazine, 1,2,3-trizole and 1,2,4-triazole. In some embodiments, an unsubstituted or a substituted heterocyclyl (such as a 5- or 6-membered heterocyclyl) can be substituted on an R⁹ group described herein. The heteroatoms present in an unsubstituted or a substituted heterocyclyl that can be substituted on an R⁹ group described herein can vary, and include O (oxygen), S (sulfur) and N (nitrogen). Exemplary unsubstituted or a substituted heterocyclyls that can be present on an R⁹ group described herein include morpholine, piperidine, piperazine, pyrrolidine, 1,2,4-oxadiazol-5(2H)-one, 1,2,4-oxadiazol-5(4H)-one, 2,4-dihydro-3H-1,2,4-triazol-3-one, azetidine and oxetane. The substituted heteroaryls and/or substituted heterocyclyls that can be substituted on an R⁹ group described herein can be substituted with one or more moieties (for example, 1, 2 or 3 moieties) such as those described herein for “optionally substituted.” In some embodiments, the substituted heteroaryls and/or substituted heterocyclyls that can be substituted on an R⁹ group described herein can be substituted with halogen, amino, an unsubstituted C₁₋₄ alkyl, an unsubstituted C₁₋₄ haloalkyl (for example, CF₃, CHF₂, CH₂F) and/or an unsubstituted C₁₋₄ alkoxy. Suitable halogens, unsubstituted C₁₋₄ alkyls, unsubstituted C₁₋₄ haloalkyls and/or unsubstituted C₁₋₄ alkoxys are described herein, such as those described in this paragraph.

In some embodiments, R¹¹ can be hydrogen; p can be 1; and R¹² can be —C(═O)—O—CR^{13A}R^{13B}—R¹⁴. In other embodiments, R¹¹ can be an unsubstituted C₁₋₄ alkyl; p can be 1; and R¹² can be —C(═O)—O—CR^{13A}R^{13B}—R¹⁴. In some embodiments, including those of this paragraph, R^{13A} can be hydrogen. In other embodiments, including those of this paragraph, R^{13A} can be unsubstituted C₁₋₄ alkyl. In some embodiments, including those of this paragraph, R^{13B} can be hydrogen. In other embodiments, including those of this paragraph, R^{13B} can be unsubstituted C₁₋₄ alkyl. Examples of unsubstituted C₁₋₄ alkyls include methyl, ethyl, n-propyl, iso-propyl, n-butyl, iso-butyl, sec-butyl and tert-butyl.

In some embodiments, R¹¹ can be hydrogen; p can be 1; and R¹² can be —C(═O)—O—CHR^{13B}—R¹⁴. In other embodiments, R¹¹ can be an unsubstituted C₁₋₄ alkyl; p can be 1; and R¹² can be —C(═O)—O—CHR^{13B}—R¹⁴. In some embodiments, R¹¹ can be methyl; p can be 1; and R¹² can be —C(═O)—O—CR^{13A}R^{13B}—R¹⁴. In some embodiments, R¹¹ can be methyl; p can be 1; and R¹² can be —C(═O)—O—CHR^{13B}—R¹⁴. In some embodiments of this paragraph, R^{13B} can be unsubstituted C₁₋₄ alkyl, such as methyl, ethyl, n-propyl, iso-propyl, n-butyl, iso-butyl, sec-butyl and tert-butyl.

A variety of moieties can be present for R¹². In some embodiments, R¹² can be —C(═O)—O—CH₂—R¹⁴. In some embodiments, R¹² can be —C(═O)—O—CH(an unsubstituted C₁₋₄ alkyl)-R¹⁴. Suitable unsubstituted C₁₋₄ alkyls are provided herein. In some embodiments, can be —C(═O)—O—CH(CH₃)—R¹⁴.

In some embodiments, R¹¹ can be hydrogen; p can be 0; and R¹² can be —R¹⁴. In other embodiments, R¹¹ can be an unsubstituted C₁₋₄ alkyl; p can be 0; and R¹² can be —R¹⁴.

Various moieties can be present for R¹⁴. In some embodiments, R¹⁴ can be a phosphate. In other embodiments, R¹⁴ can be a phosphate connected via a lower alkylene. For example, R¹⁴ can be

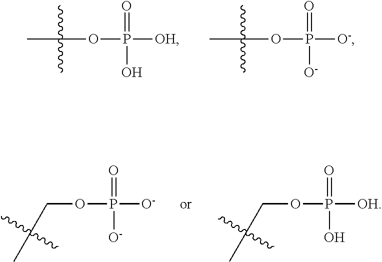

As with a phosphate, R¹⁴ can be —O-linked α-amino acid or an —O-linked α-amino acid linked via a lower alkylene. In some embodiments, R¹⁴ can be —O-linked α-amino acid. In other embodiments, R¹⁴ can be —CH₂—O-linked α-amino acid. Examples of α-amino acids include arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan. In some embodiments, the O-linked amino acid can be

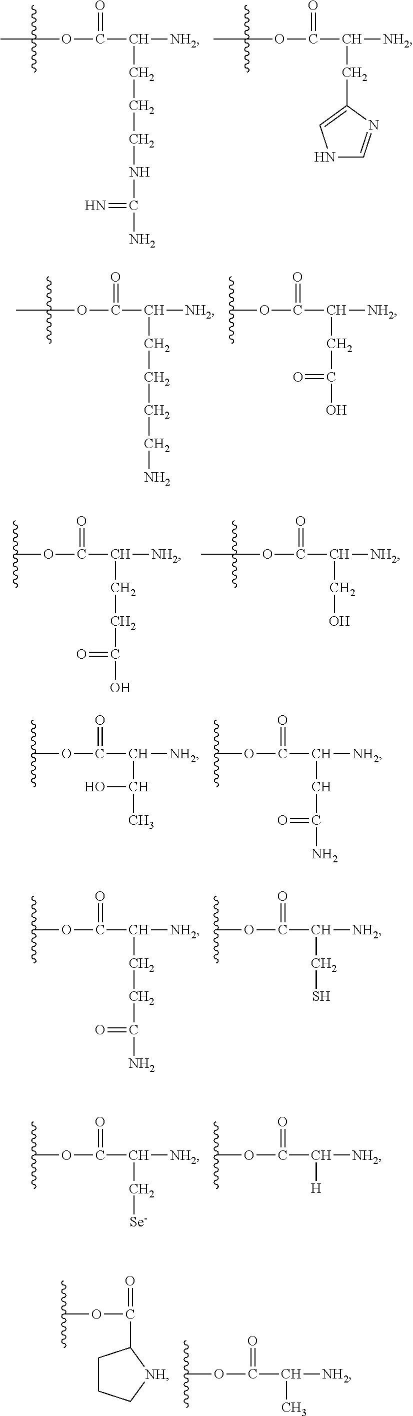

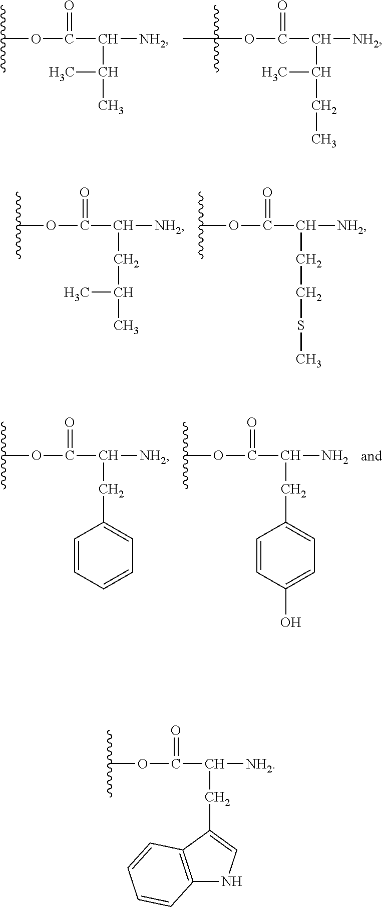

Those skilled in the art understand that an α-amino acid, except for glycine, has a chiral center. In some embodiment, the chiral center of an α-amino acid may be a (R)-chiral center. In other embodiments, the chiral center of an α-amino acid may be a (S)-chiral center.

In some embodiments, R¹⁴ can be a hydroxy-substituted C₁₋₄ alkyl. For example, R¹⁴ can be —CH₂—OH, —CH₂CH₂—OH, —CH₂CH₂CH₂—OH, —CH₂CH₂CH₂CH₂—OH or —CH(OH)—CH₂CH₃, —CH(CH₃)—CH₂OH In other embodiments, R¹⁴ can be —O—(C═O)—CH(OH)—CH(OH)—C(═O)OH. In still other embodiments, R¹⁴ can be —O—(C═O)—CH₂—CH(OH)—C(═O)OH. In yet still other embodiments, R¹⁴ can be —O—(C═O)—CH═CH—C(═O)OH. In some embodiments, R¹⁴ can be —(C═O)-(an unsubstituted or a substituted monocyclic heterocyclyl). A variety of monocyclic heterocyclyls can be part of —(C═O)-(an unsubstituted or a substituted monocyclic heterocyclyl) for R¹⁴. For example, the monocyclic heterocyclyl can be a 5- or 6-membered monocyclic heterocyclyl. A variety of heteroatoms can be in the ring of a monocyclic heterocyclyl, such as N (nitrogen), O (oxygen) and S (sulfur). Additionally, the number of heteroatoms present in the ring of a monocyclic heterocyclyl can vary. For example, a monocyclic heterocyclyl of —(C═O)-(an unsubstituted or a substituted monocyclic heterocyclyl) for R¹⁴ can include 1 or 2 heteroatoms. In some embodiments, the monocyclic heterocyclyl of —(C═O)-(an unsubstituted or a substituted monocyclic heterocyclyl) for R¹⁴ can be a 5- or 6-membered monocyclic heterocyclyl that includes 1 or 2 nitrogens and optionally 1 heteroatom selected from O (oxygen) and S (sulfur). A non-limiting list of monocyclic heterocyclyl include pyrrolidinyl, imidazolidinyl, piperidinyl, piperazinyl, tetrahydro-2H-pyranyl, tetrahydro-2H-thiopyranyl, morpholinyl and thiomorpholinyl.

In some embodiments, a compound of Formula (I), or a pharmaceutically acceptable salt thereof, can be where n can be 1; Z¹ is —C(═O)—; R¹ can be

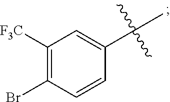 [SMILES: *c1ccc(Br)c(C(F)(F)F)c1]

R² can be an unsubstituted C₁₋₄ alkyl (such as methyl); R³, R⁴, R⁵, R⁶ and R⁷ can each be hydrogen; R¹ can be —NHR^{10B}; R⁹ can be a substituted phenyl, wherein the phenyl is substituted with —C(═O)NHR¹²; and R^{10B} can be an unsubstituted C₂₋₈ alkenyl. In some embodiments, a compound of Formula (I), or a pharmaceutically acceptable salt thereof, can be where n can be 1; Z¹ is —C(═O)—; R¹ can be

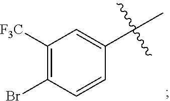 [SMILES: *c1ccc(Br)c(C(F)(F)F)c1]

R² can be an unsubstituted C₁₋₄ alkyl (such as methyl); R³, R⁴, R⁵, R⁶ and R⁷ can each be hydrogen; R⁸ can be —NHR^{10B}; R⁹ can be a substituted phenyl, wherein the phenyl is substituted with —C(═O)NHR¹²; and R^{10B} can be an unsubstituted C₂₋₈ alkynyl. In some embodiments, a compound of Formula (I), or a pharmaceutically acceptable salt thereof, can be where n can be 1; Z¹ is —C(═O)—; R¹ can be

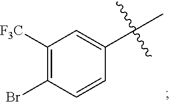 [SMILES: *c1ccc(Br)c(C(F)(F)F)c1]

R² can be an unsubstituted C₁₋₄ alkyl (such as methyl); R³, R⁴, R⁵, R⁶ and R⁷ can each be hydrogen; R⁸ can be —NHR^{10B}; R⁹ can be a substituted phenyl, wherein the phenyl is substituted with —C(═O)NHR¹²; and R^{10B} can be an unsubstituted C₂₋₈ alkenyl or an unsubstituted C₂₋₈ alkynyl.

Examples of compounds of Formula (I), including pharmaceutically acceptable salts thereof, include:

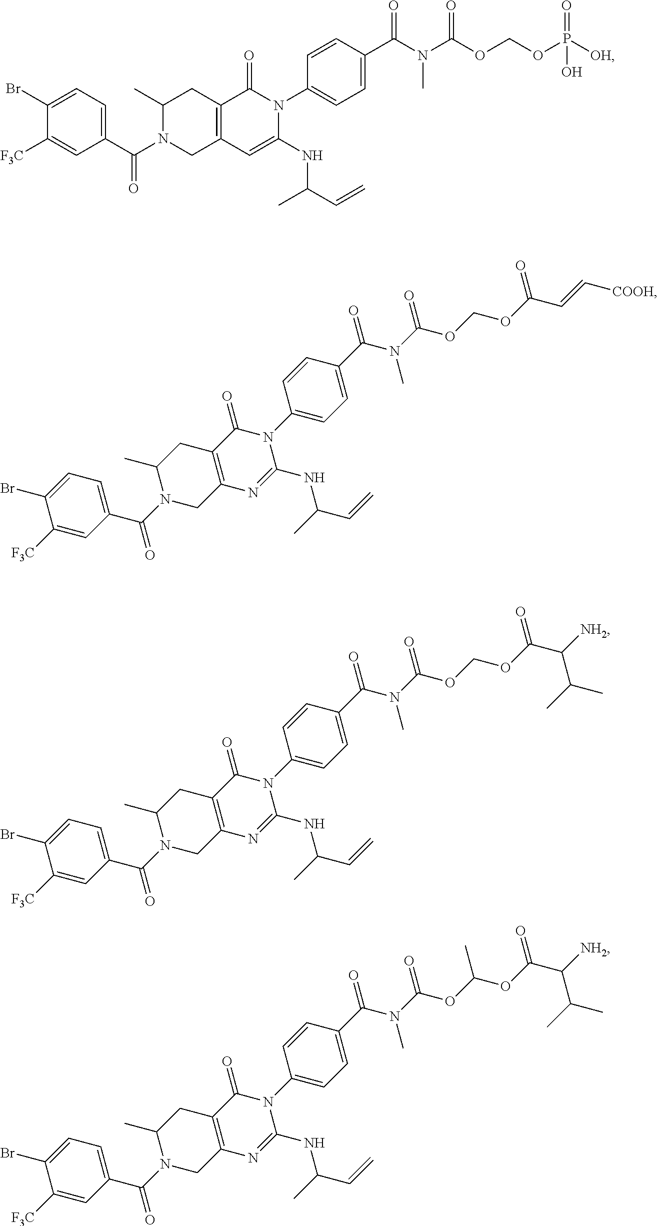 [SMILES: C=CC(C)Nc1cc2c(c(=O)n1-c1ccc(C(=O)N(C)C(=O)OCOP(=O)(O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)/C=C/C(=O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)C(N)C(C)C)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OC(C)OC(=O)C(N)C(C)C)cc1)CC(C)N(C(=O)c1ccc(Br)c(C(F)(F)F)c1)C2]

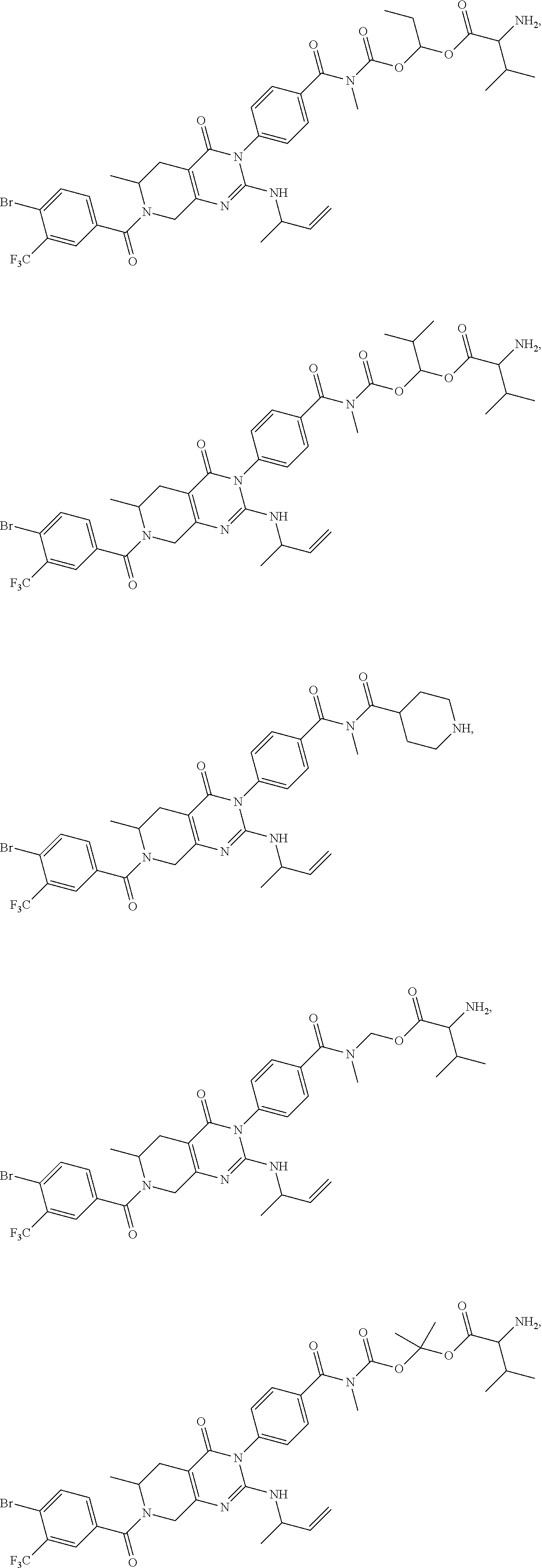

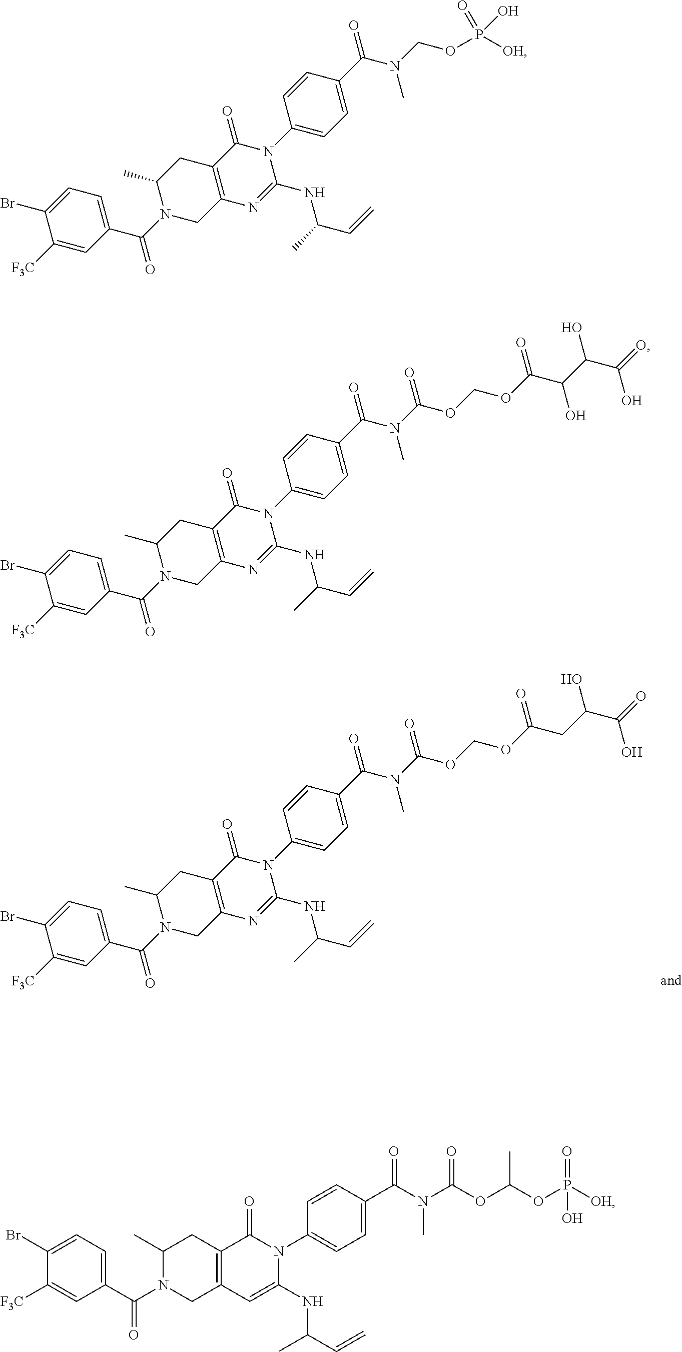 [SMILES: C=C[C@H](C)Nc1nc2c(c(=O)n1-c1ccc(C(=O)N(C)COP(=O)(O)O)cc1)C[C@@H](C)N(C(=O)c1ccc(Br)c(C(F)(F)F)c1)C2 ; C=CC(C)Nc1nc2c(c(=O)n1-c1ccc(C(=O)N(C)C(=O)OCOC(=O)C(O)C(O)C(=O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)CC(O)C(=O)O)cc1)CC(C)N(C(=O)c1ccc(Br)c(C(F)(F)F)c1)C2 ; C=CC(C)Nc1cc2c(c(=O)n1-c1ccc(C(=O)N(C)C(=O)OC(C)OP(=O)(O)O)cc1)CC(C)N(C(=O)c1ccc(Br)c(C(F)(F)F)c1)C2]

or a pharmaceutically acceptable salt of any of the foregoing.

Additional examples of compounds of Formula (I), including pharmaceutically acceptable salts thereof, include:

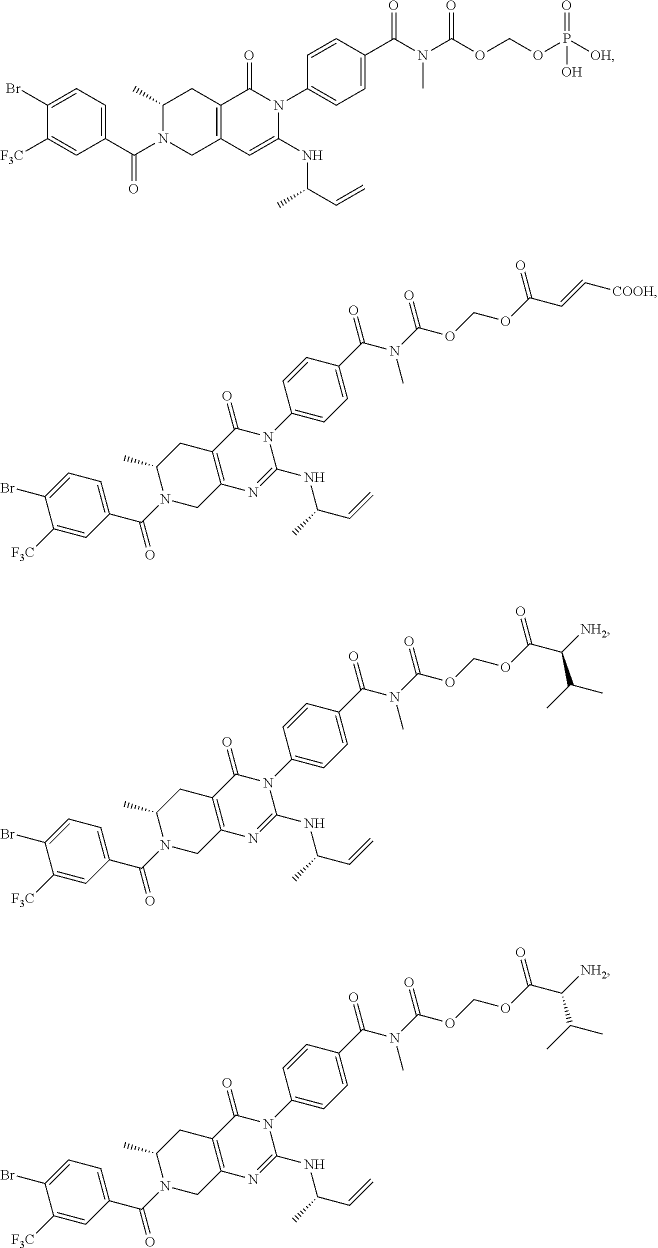 [SMILES: C=C[C@H](C)Nc1cc2c(c(=O)n1-c1ccc(C(=O)N(C)C(=O)OCOP(=O)(O)O)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)/C=C/C(=O)O)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)[C@@H](N)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)[C@H](N)C(C)C)cc1)C[C@@H](C)N(C(=O)c1ccc(Br)c(C(F)(F)F)c1)C2]

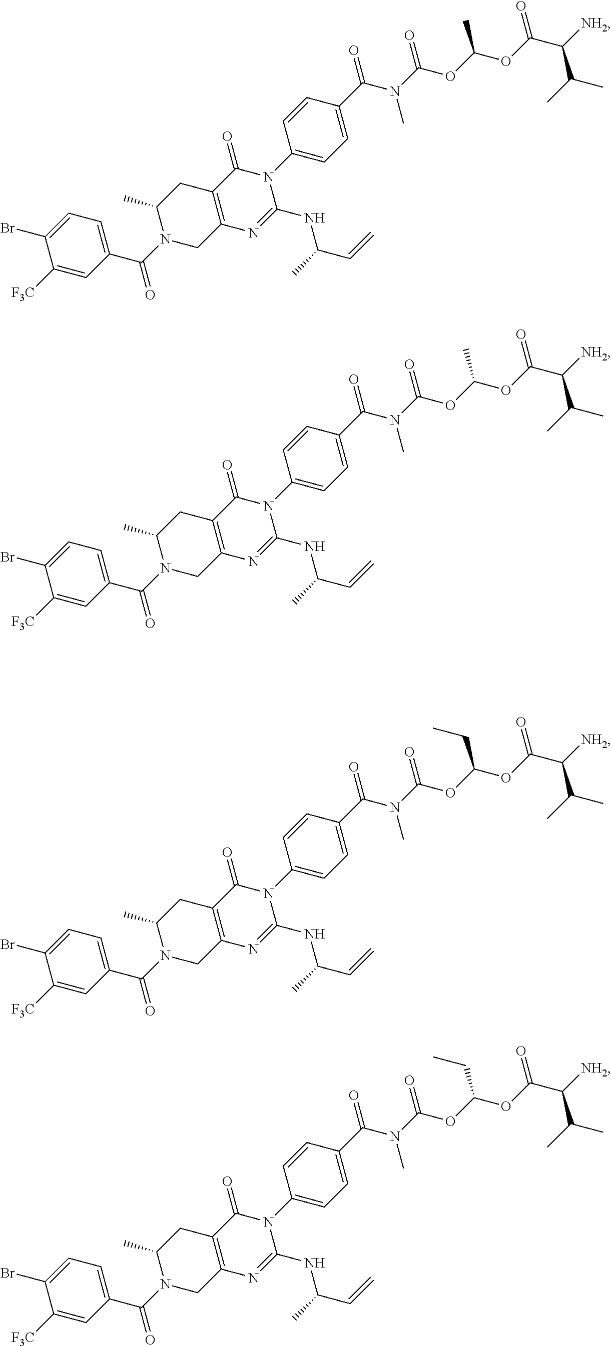

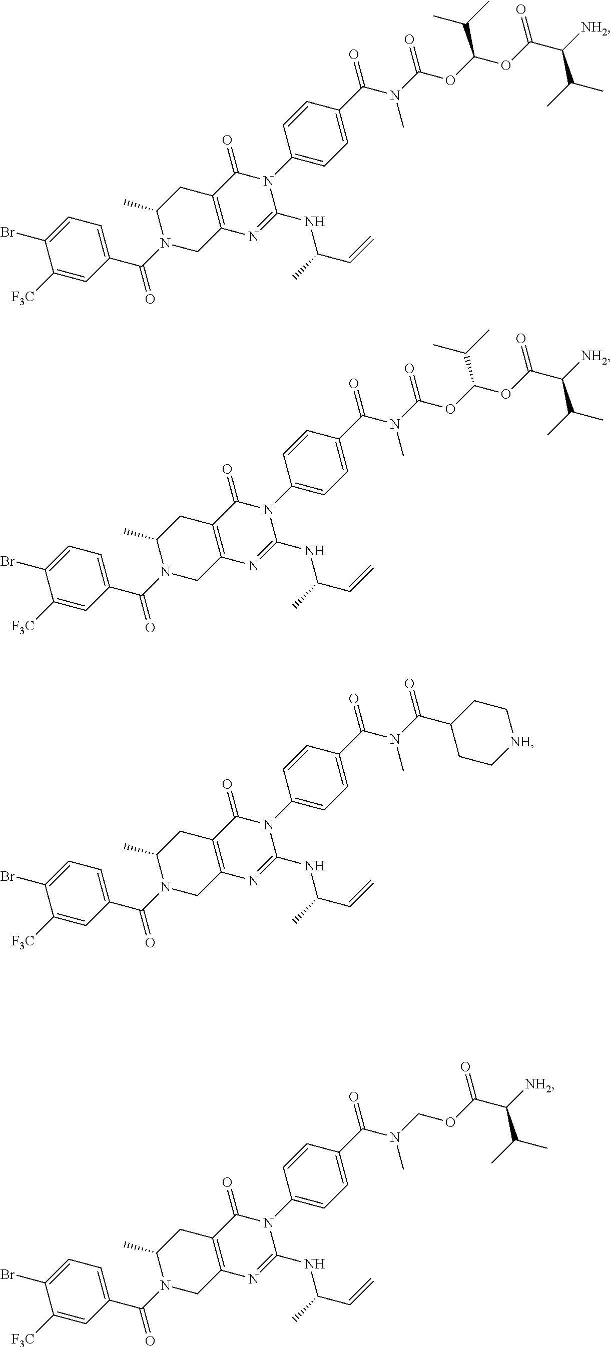 [SMILES: C=C[C@H](C)Nc1nc2c(c(=O)n1-c1ccc(C(=O)N(C)C(=O)O[C@H](OC(=O)[C@@H](N)C(C)C)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)O[C@@H](OC(=O)[C@@H](N)C(C)C)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)C3CCNCC3)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)COC(=O)[C@@H](N)C(C)C)cc1)C[C@@H](C)N(C(=O)c1ccc(Br)c(C(F)(F)F)c1)C2]

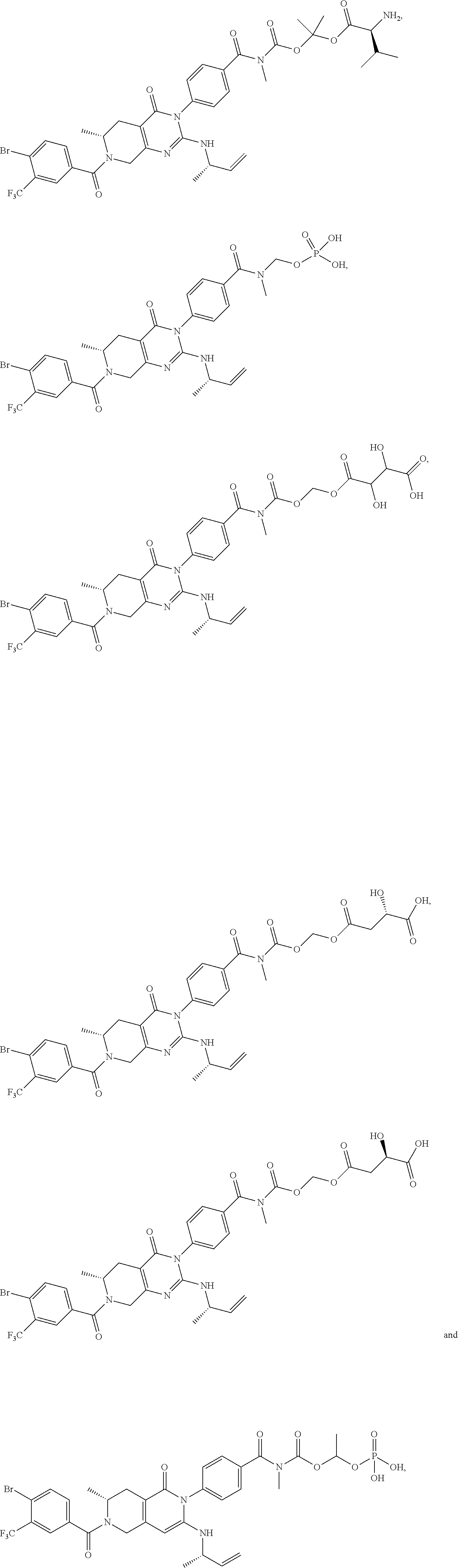

or a pharmaceutically acceptable salt of any of the foregoing.

### Synthesis

Compounds of Formula (I) along with those described herein may be prepared in various ways. General synthetic routes for preparing compounds of Formula (I) are shown and described herein along with some examples of starting materials used to synthesize compounds described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

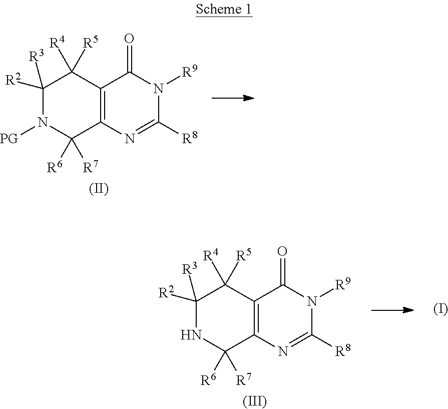

Compounds of Formula (I) can be prepared from an intermediate of Formula (II), in which PG represents an amino protecting group such as Boc. The PG group can be cleaved from a compound of Formula (II) using methods known in the art. For example, when PG represents a Boc group, PG can be cleaved using acidic conditions, for example, in the presence of HCl in a suitable solvent (such as 1,4-dioxane) or in the presence of copper triflate. The coupling of the intermediate of Formula (III) with a suitable agent can afford a compound of Formula (I), along with pharmaceutically acceptable salts thereof. As an example, compounds of Formula (I), along with pharmaceutically acceptable salts thereof, wherein Z¹ represents —NH—C(═O)— and n=1, can be obtained by reacting a compound of Formula (III) with a phenyl carbamate of general formula R¹—NH—C(═O)—O-phenyl or with an isocyanate of general formula R¹—N═C═O, in the presence of a suitable base in a suitable solvent. An example of a suitable base is triethylamine, and an example of suitable solvent is acetonitrile.

Other compounds of Formula (I), along with pharmaceutically acceptable salts thereof, wherein Z¹ represents —C(═O)— and n=1, can be obtained by reacting a compound of Formula (III) with an acyl chloride of general formula R¹—C(═O)—Cl in the presence of a base in a suitable solvent. Additional compounds of Formula (I) and pharmaceutically acceptable salts thereof, wherein Z¹ represents —C(═O)— and n=1, can be obtained by reacting compound of Formula (III) with a carboxylic acid of general formula R¹—C(═O)—OH in the presence of an amide coupling agent (such as HATU) in a suitable solvent. Further compounds of Formula (I), along with pharmaceutically acceptable salts thereof, can be prepared from a compound of Formula (III) using methods known in the art.

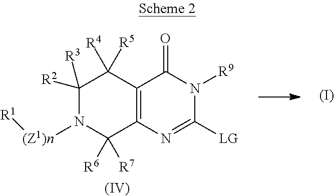

Compounds of Formula (I), including pharmaceutically acceptable salts thereof, can also be prepared from an intermediate of Formula (IV), in which LG represents a leaving group (such as sulfhydryl, methylsulfoxide or halo, in particular chloro or bromo). A compound of Formula (I) in which R¹ represents —NR^{10A}R^{10B} can be prepared from a compound of Formula (IV) in which LG represents methylsulfoxide by reacting an amine of Formula HNR^{10A}R^{10B}, in the presence of a base (such as diisopropylethylamine (DIPEA) or sodium bicarbonate) in a suitable solvent (such as 1,4-dioxane or acetonitrile), optionally in the presence of a catalyst (for example, DMAP). A compound of Formula (I) in which R¹ represents —NR^{10A}R^{10B} can be prepared from a compound of Formula (IV) in which LG represents chloro by reacting an amine of Formula HNR^{10A}R^{10B}, in the presence of a base (for example, triethylamine, sodium bicarbonate or DIPEA) in a suitable solvent (such as acetonitrile, n-butanol or dioxane), optionally in the presence of a catalyst, such as DMAP.

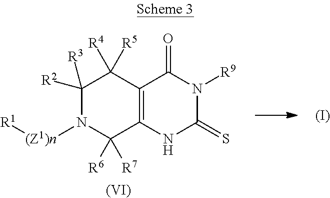

Compounds of Formula (I) in which R⁸ represents —NR^{10A}R^{10B} can be prepared from an intermediate of Formula (VI) and an amine of general formula HNR^{10A}R^{10B}, in the presence of tert-Butyl hydroperoxide (TBHP) in a suitable solvent (such as acetonitrile).

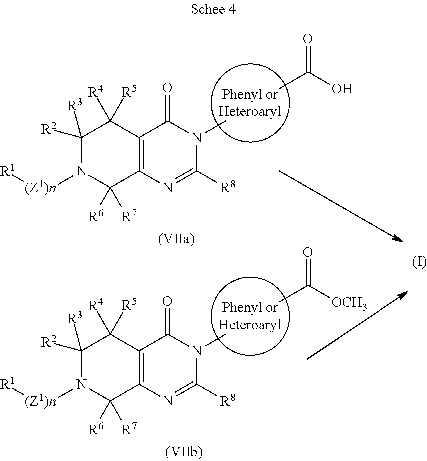

Compounds of Formula (I), including pharmaceutically acceptable salts thereof, in which R⁹ represents a phenyl substituted with —C(═O)NHR¹² can be prepared from an acid intermediate of general formula (VIIa) and an amine of Formula NH₂—R¹², using a peptide coupling agent (such as CDI) in the presence of a base (for example, DBU) in a suitable solvent, such as acetonitrile or DMF. Compounds of Formula (I), along with pharmaceutically acceptable salts thereof, in which R⁹ represents a phenyl substituted with —C(═O)NHR¹² can be prepared from an ester intermediate of Formula (VIIb) and an amine of general formula NH₂—R¹² in a suitable solvent (such as acetonitrile).

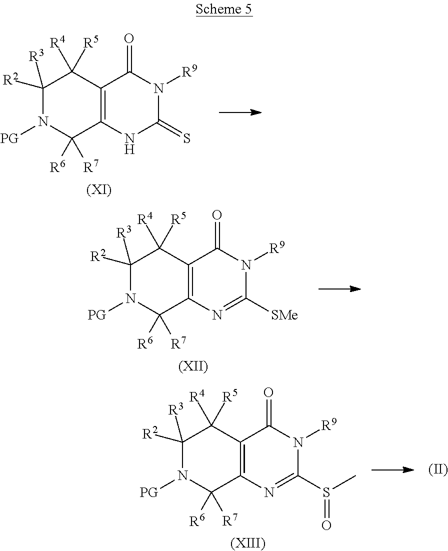

Intermediate of Formula (II) in which R⁸ represents —NR^{10A}R^{10B} can be prepared from a compound of Formula (XI) using methyl iodide or methyl bromide, in the presence of a base, such as DBU, in a suitable solvent, such as DMF, to afford an intermediate of Formula (XII). Oxidation of an intermediate of Formula (XII) to a sulfoxide intermediate of Formula (XIII) can be achieved by a treatment with an oxidative agent (such as m-CPBA) in the presence of MgSO₄ and NaOAc in a suitable solvent (such as dichloromethane). Treatment of intermediate of Formula (XIII) with an amine of general formula HNR^{10A}R^{10B} in the presence of a base (such as DIPEA) in the presence of a catalyst (for example, DMAP) in a suitable solvent (such as 1,4-dioxane) can afford an intermediate of Formula (II) in which R¹ represents —NR^{10A}R^{10B}.

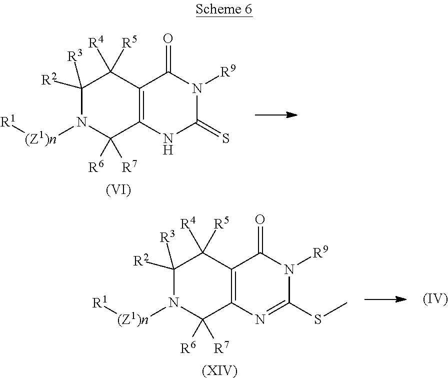

Intermediates of Formula (IV) in which the leaving group LG represents a methylsulfoxide can be prepared from an intermediate of Formula (VI) using methyl iodide or methyl bromide, in the presence of a base (for example, DBU) in a suitable solvent, such as DMF, to afford an intermediate of Formula (XIV). Oxidation of an intermediate of Formula (XIV) to a sulfoxide intermediate of Formula (IV) can be achieved using an oxidative agent, such as m-CPBA, in the presence of MgSO₄ and NaOAc in a suitable solvent, such as dichloromethane.

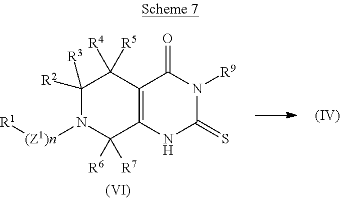

Intermediates of Formula (IV) in which the leaving group LG represents a chloro can be prepared from an intermediate of Formula (VI) using thiophosgene in a suitable solvent (such as 1,4-dioxane).

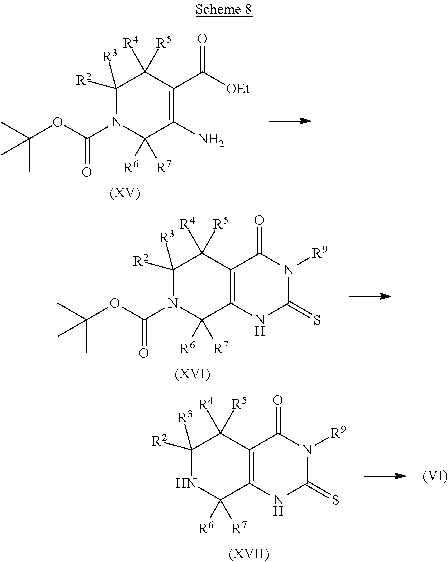

Intermediate of Formula (VI) can be prepared from an intermediate of Formula (XV) in the presence of a strong base, such as NaH, in a suitable solvent (for example, THF or 2-methylTHF) followed by the subsequent addition of an isothiocyanate of general formula R⁹—NCS to afford an intermediate of Formula (XVI). The Boc group of an intermediate of Formula (XVI) can be obtained in the presence of an acid (such as HCl or TFA) in a suitable solvent (for example, 1,4-dioxane) to afford an intermediate of Formula (XVII). Intermediates of Formula (VI) can be prepared from an intermediate of Formula (XVII) following several conditions known to those skilled in the art. For example, compounds of Formula (XVII), wherein Z¹ represents —NH—C(═O)— and n=1, can be obtained by reacting a compound of Formula (XVII) with a phenyl carbamate of general formula R¹—NH—C(O)—O— phenyl or with an isocyanate of general formula R¹—N═C═O, in the presence of a suitable base in a suitable solvent. An example of a suitable base is triethylamine, and an example of suitable solvent is acetonitrile or dichloromethane.

Further compounds of Formula (VI) wherein Z¹ represents —C(O)— and n=1, can be obtained by reacting a compound of Formula (XVII) with an acyl chloride of general formula R¹—C(═O)—Cl in the presence of a base in a suitable solvent, including those bases and solvents described herein and/or known to those skilled in the art. Compounds of Formula (VI), wherein Z¹ represents —C(═O)— and n=1, can be obtained by reacting compound of Formula (XVII) with a carboxylic acid of general formula R¹—C(═O)—OH in the presence of an amide coupling agent (such as HATU) in a suitable solvent. Additional compounds of Formula (VI) can be prepared from a compound of Formula (XVII) using methods known in the art.

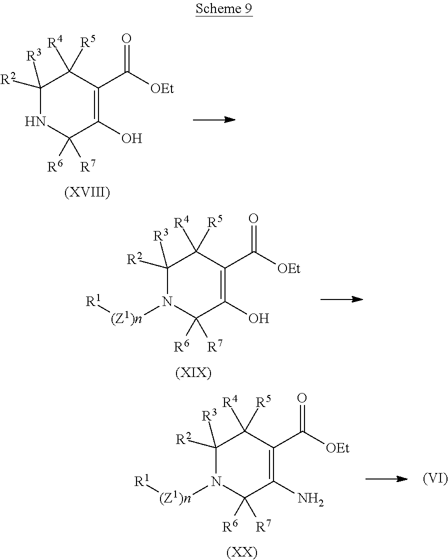

An intermediate of Formula (VI) can be prepared from an intermediate of Formula (XVIII) following other conditions known in the art, similar to the conditions used to convert an intermediate of Formula (XVII) to an intermediate for Formula (VI). For example, intermediates of Formula (XIX) wherein Z¹ represents —C(═O)— and n=1, can be obtained by reacting a compound of Formula (XVIII) with an acyl chloride of general formula R¹—C(═O)—Cl in the presence of a base in a suitable solvent. Additional compounds of Formula (XIX), wherein Z¹ represents —C(═O)— and n=1, can be obtained by reacting compound of Formula (XVIII) with a carboxylic acid of general formula R¹—C(═O)—OH in the presence of an amide coupling agent (such as HATU) in a suitable solvent. Suitable solvents are known to those skilled in the art and/or described herein.

Intermediates of Formula (XX) can be prepared from an intermediate of Formula (XI) in the presence of ammonium acetate, in a suitable solvent (such as ethanol). Intermediate of Formula (VI) can be prepared from an intermediate of Formula (XX) in the presence of a strong base (for example, NaH) in a suitable solvent (such as THF or 2-methylTHF) followed by the addition of an isothiocyanate of general formula R⁹—NCS. An intermediate of Formula (XX) can be treated with thiophosgene/NMM in a suitable solvent, such as dichloromethane, to afford an intermediate isothiocyanate, which can be converted to an intermediate of Formula (VI) by using an amine of general formula NH₂—R⁹, in the presence of a base, such as triethylamine, in a suitable solvent (such as acetonitrile).

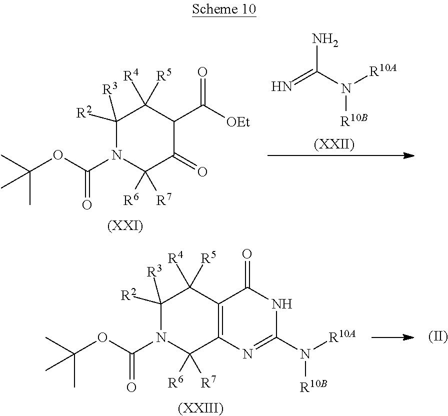

Intermediates of Formula (II) in which R⁸ represents —NR^{10A}R^{10B} and the protecting group PG represents Boc, can be prepared from an intermediate of Formula (XXI) using a guanidine derivative of Formula (XXII), in the presence of a base, such as DBU, in a suitable solvent (such as acetonitrile) to afford an intermediate of Formula (XXIII). An intermediate of Formula (XXIII) can be converted in the intermediate of Formula (II) using methods known in the art. As an example, an intermediate of formula (XXIII) can be reacted with an aryl or heteroaryl boronic acid of general formula R⁹—B(OH)₂, in the presence of TMEDA and Cu(OAc)₂ to afford an intermediate of Formula (II) in which R⁹ represents a substituted phenyl, a substituted monocyclic heteroaryl or a substituted fused-bicyclic heteroaryl.

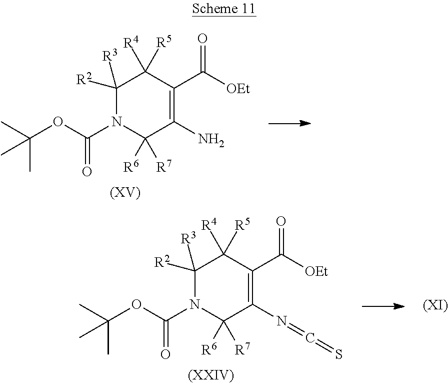

Intermediates of Formula (XI) can be obtained from an intermediate of Formula (XV) using methods known in the art, for example by treating an intermediate of Formula (XV) with thiophosgene and NMM in a suitable solvent (such as dichloromethane). Treatment of an intermediate of Formula (XXIV) with an amine of general formula R⁹—NH₂ affords an intermediate of Formula (XI) in which PG represents a Boc group.

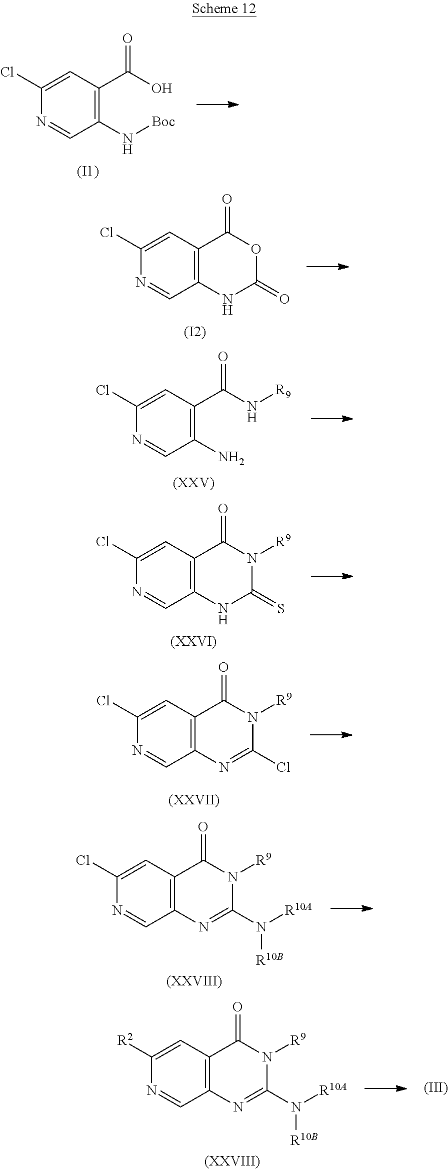

Intermediates of Formula (III) in which R³, R⁴, R⁵, R⁶ and R⁷ each represent hydrogen, and in which R¹ represents —NR^{10A}R^{10B}, can be prepared from a chloro-N-Boc-aminopyridinecarboxylic acid intermediate of Formula (I1) using a base (such as triethylamine) in the presence of 2-chloro-N-methylpyridinium iodide in a suitable solvent (for example, acetonitrile) to afford an intermediate of Formula (I2). An intermediate of Formula (I2) can be converted to an intermediate of Formula (XXV) using an amine of general formula R⁹—NH₂, in a suitable solvent (for example, acetic acid). Reaction of an intermediate of Formula (XXV) with thiocarbonyldiimidazole in DMF can afford the thio intermediate of Formula (XXVI), which can be converted in an intermediate of Formula (XXVII) using thiophosgene in a suitable solvent (such as 1,4-dioxane). Treatment of an intermediate of Formula (XXVII) with an amine of general formula NR^{10A}R^{10B} can afford an intermediate of Formula (XXVIII). An intermediate of Formula (XXVIII) can be reacted with an organometallic derivative (such as a tin derivative of general formula R²—Sn(n-Bu)₃). A n intermediate of Formula (XXVIII) can be converted to an intermediate of Formula (III) in which R³, R⁴, R⁵, R⁶ and R⁷ each can be hydrogen, and in which R¹ represents —NR^{10A}R^{10B} by hydrogenation using H₂ in the presence of a catalyst (such as Pt/C) in a mixture of solvents (for example, acetic acid/THF/ethanol). In the instance where R² can be an unsaturated group, such as an alkene, the R² can be converted to another R² group, such as an alkyl, by hydrogenation.

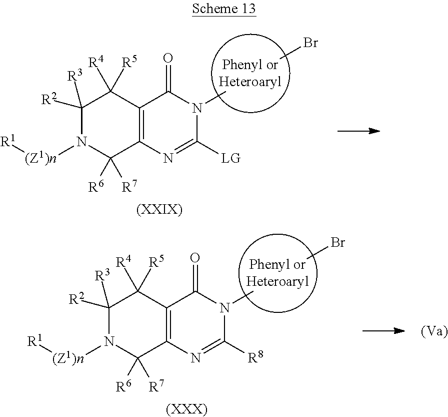

Intermediates of Formula (Va), in which R⁸ represents —NR^{10A}R^{10B}, can be prepared from an intermediate of Formula (XXIX), in which LG represents a leaving group (such as sulfhydryl, methylsulfoxide or halo, in particular chloro of bromo). Intermediates of Formula (XXIX) can be reacted with an amine of general formula HNR^{10A}R^{10B}, in the presence of a base (for example, triethylamine) in a suitable solvent, such as acetonitrile, to afford an intermediate of Formula (XXX). The conversion of a bromo intermediate of Formula (XXX) to a boronic ester intermediate of Formula (Va) can be achieved using bis(pinacolato)diboron in the presence of a catalyst (such as Pd(dppf)Cl₂) in the presence of a base, such as KOAc, in a suitable solvent (for example, 1,4-dioxane).

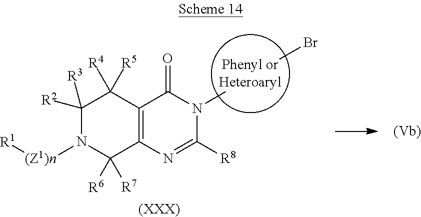

Intermediates of Formula (Vb) can be prepared from an intermediate of Formula (XXX) using bis(pinacolato)diboron, in the presence of a base (such as potassium acetate and 1,1′-bis(diphenylphosphino)ferrocene-palladium(II)dichloride dichloromethane complex) in a suitable solvent, such as 1,4-dioxane, to obtain an intermediate of Formula (Vb).

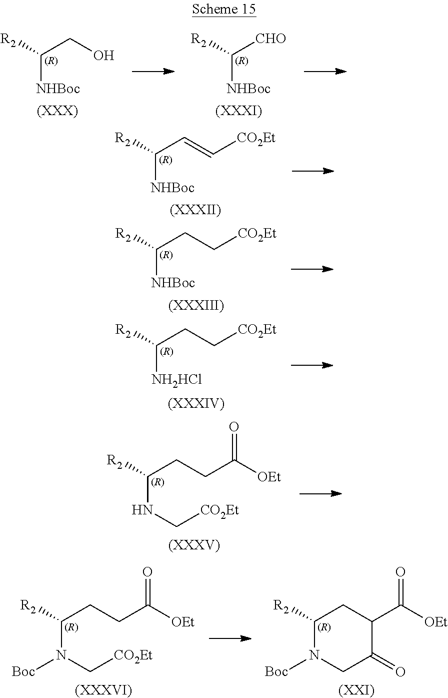

An alternative approach towards the chiral synthesis of compounds of Formula (XXI) is provided in Scheme 15. Oxidation of a chiral, boc-protected aminoalcohol of Formula (XXX), for example, via Swern oxidation, leads to aldehydes of Formula (XXXI). Subsequent formation of an unsaturated ester, for example, via the Wittig reaction (compounds of Formula (XXXII)), and then reduction of the resultant double bond (e.g., Pd/C) can afford compounds of Formula (XXXIII). Deprotection of the boc group and alkylation with ethyl 2-bromoacetate can provide compounds of Formula (XXXV). Boc protection of compounds of Formula (XXXV) followed by Dieckmann condensation can provide to compounds of Formula (XXI).

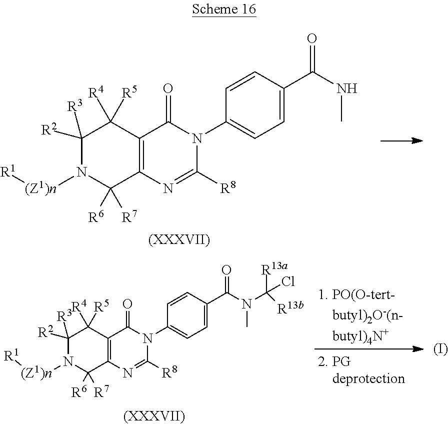

A compound of formula (I), where n is 1, Z¹ is —C(═O)— and R¹⁴ is —OP(═O)(OH)₂ can be synthetized according to Scheme 16 by reacting a compound of Formula (XXXVII) with a chloroformate ClCOOR^{13a}R^{13b}Cl, in presence of a base such as LiHMDS in a suitable solvent like THF. Subsequent reaction with a protected phosphate derivative, such as tetra-n-butylammonium di-tert-butylphosphate, followed by deprotection of the protecting group, using, for example, TFA in DCM to remove the tert-butyl group, can lead to a compound of Formula (I) (along with pharmaceutically acceptable salts thereof).

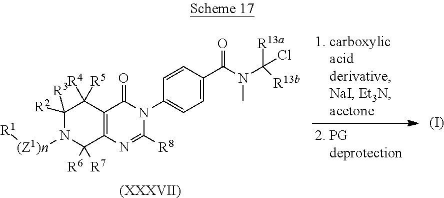

A compound of Formula (I) (including pharmaceutically acceptable salts thereof), where n is 1, Z¹ is —C(═O)— and R¹⁴ is —O—(C═O)—CH═CH—C(═O)OH or an —O-linked α-amino acid can be prepared according to Scheme 17 by reacting a compound of Formula (XXXVII) with a carboxylic acid derivative, in presence of NaI and a base (such as Et₃N), in a suitable solvent (such as acetone). In case of R¹⁴ being —O—(C═O)—CH═CH—C(═O)OH, the carboxylic acid derivative can be (E)-4-(tert-Butoxy)-4-oxobut-2-enoic acid, which is then deprotected at the appropriate point of the synthesis using formic acid. In case of R¹⁴ being an —O-linked α-amino acid, a N-protected amino acid can be reacted with a compound of Formula (XXXVII), and the N-protecting group can be removed in the final step to obtain a compound of Formula (I) (including pharmaceutically acceptable salts thereof). When the N-protecting group is Boc, the deprotection may occur in presence of HCl in dioxane, to provide a compound of Formula (I) as a HCl salt.

### Pharmaceutical Compositions

Some embodiments described herein relate to a pharmaceutical composition, that can include an effective amount of a compound described herein (e.g., a compound, or a pharmaceutically acceptable salt thereof, as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a HBV and/or HDV infection that can include administering to a subject identified as suffering from the HBV and/or HDV infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a HBV and/or HDV infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a HBV and/or HDV infection.

Some embodiments disclosed herein relate to a method of treating a HBV and/or HDV infection that can include contacting a cell infected with the HBV and/or HDV with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a HBV and/or HDV infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a HBV and/or HDV infection.

Some embodiments disclosed herein relate to a method of inhibiting replication of HBV and/or HDV that can include contacting a cell infected with the HBV and/or HDV with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of HBV and/or HDV. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of HBV and/or HDV.

In some embodiments, the HBV infection can be an acute HBV infection. In some embodiments, the HBV infection can be a chronic HBV infection.

Some embodiments disclosed herein relate to a method of treating liver cirrhosis that is developed because of a HBV and/or HDV infection that can include administering to a subject suffering from liver cirrhosis and/or contacting a cell infected with the HBV and/or HDV in a subject suffering from liver cirrhosis with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating liver cirrhosis with an effective amount of the compound, or a pharmaceutically acceptable salt thereof. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating liver cirrhosis.

Some embodiments disclosed herein relate to a method of treating liver cancer (such as hepatocellular carcinoma) that is developed because of a HBV and/or HDV infection that can include administering to a subject suffering from the liver cancer and/or contacting a cell infected with the HBV and/or HDV in a subject suffering from the liver cancer with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating liver cancer (such as hepatocellular carcinoma). Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating liver cancer (such as hepatocellular carcinoma).

Some embodiments disclosed herein relate to a method of treating liver failure that is developed because of a HBV and/or HDV infection that can include administering to a subject suffering from liver failure and/or contacting a cell infected with the HBV and/or HDV in a subject suffering from liver failure with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating liver failure. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating liver failure.

Various indicators for determining the effectiveness of a method for treating an HBV and/or HDV infection are also known to those skilled in the art. Examples of suitable indicators include, but are not limited to, a reduction in viral load indicated by reduction in HBV DNA (or load) (e.g., reduction <10⁵ copies/mL in serum), HBV surface antigen (HBsAg) and HBV e-antigen (HBeAg), a reduction in plasma viral load, a reduction in viral replication, an increase in the rate of sustained viral response to therapy, an improvement in hepatic function, and/or a reduction of morbidity or mortality in clinical outcomes.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

The term “effective amount” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In some embodiments, an effective amount of a compound, or a pharmaceutically acceptable salt thereof, as described herein is an amount that is effective to achieve a sustained virologic response, for example, a sustained viral response 12 month after completion of treatment. A non-limiting list of potential advantages of compounds described herein (such as a compound of Formula (I), and pharmaceutically acceptable salts thereof) include improvement of one or more pharmacokinetic parameters (for example, Cmax, Tmax, AUC, half-life (t_{1/2}) and bioavailability (F)) improved stability, increased safety profile, increased efficacy, increased binding to the target and/or increased specificity for the target (for example, a cancer cell).

Subjects who are clinically diagnosed with a HBV and/or HDV infection include “naïve” subjects (e.g., subjects not previously treated for HBV and/or HDV) and subjects who have failed prior treatment for HBV and/or HDV (“treatment failure” subjects). Treatment failure subjects include “non-responders” (subjects who did not achieve sufficient reduction in ALT (alanine aminotransferase) levels, for example, subject who failed to achieve more than 1 log 10 decrease from base-line within 6 months of starting an anti-HBV and/or anti-HDV therapy) and “relapsers” (subjects who were previously treated for HBV and/or HDV whose ALT levels have increased, for example, ALT>twice the upper normal limit and detectable serum HBV DNA by hybridization assays). Further examples of subjects include subjects with a HBV and/or HDV infection who are asymptomatic.

In some embodiments, a compound, or a pharmaceutically acceptable salt thereof, as described herein can be provided to a treatment failure subject suffering from HBV and/or HDV. In some embodiments, a compound, or a pharmaceutically acceptable salt thereof, as described herein can be provided to a non-responder subject suffering from HBV and/or HDV. In some embodiments, a compound, or a pharmaceutically acceptable salt thereof, as described herein can be provided to a relapser subject suffering from HBV and/or HDV. In some embodiments, the subject can have HBeAg positive chronic hepatitis B. In some embodiments, the subject can have HBeAg negative chronic hepatitis B. In some embodiments, the subject can have liver cirrhosis. In some embodiments, the subject can be asymptomatic, for example, the subject can be infected with HBV and/or HDV but does not exhibit any symptoms of the viral infection. In some embodiments, the subject can be immunocompromised. In some embodiments, the subject can be undergoing chemotherapy.

Examples of agents that have been used to treat HBV and/or HDV include immunomodulating agents, and nucleosides/nucleotides. Examples of immunomodulating agents include interferons (such as IFN-α and pegylated interferons that include PEG-IFN-α-2a); and examples of nucleosides/nucleotides include lamivudine, telbivudine, adefovir dipivoxil, clevudine, entecavir, tenofovir alafenamide and tenofovir disoproxil. However, some of the drawbacks associated with interferon treatment are the adverse side effects, the need for subcutaneous administration and high cost. Potential advantages of a compound of Formula (I), or a pharmaceutically acceptable salt of any of the foregoing, can be less adverse side effects, delay in the onset of an adverse side effect and/or reduction in the severity of an adverse side effect. A drawback with nucleoside/nucleotide treatment can be the development of resistance, including cross-resistance.

Resistance can be a cause for treatment failure. The term “resistance” as used herein refers to a viral strain displaying a delayed, lessened and/or null response to an anti-viral agent. In some embodiments, a compound, or a pharmaceutically acceptable salt thereof, as described herein can be provided to a subject infected with an HBV and/or HDV strain that is resistant to one or more anti-HBV and/or anti-HDV agents. Examples of anti-viral agents wherein resistance can develop include lamivudine, telbivudine, adefovir dipivoxil, clevudine, entecavir, tenofovir alafenamide and tenofovir disoproxil. In some embodiments, development of resistant HBV and/or HDV strains is delayed when a subject is treated with a compound, or a pharmaceutically acceptable salt thereof, as described herein compared to the development of HBV and/or HDV strains resistant to other HBV and/or HDV anti-viral agents, such as those described.

### Combination Therapies

In some embodiments, a compound, or a pharmaceutically acceptable salt thereof, as described herein can be used in combination with one or more additional agent(s) for treating and/or inhibiting replication HBV and/or HDV. Additional agents include, but are not limited to, an interferon, nucleoside/nucleotide analogs, a sequence specific oligonucleotide (such as anti-sense oligonucleotide and siRNA), nucleic acid polymers (NAPs, such as nucleic acid polymers that reduce HBsAg levels including STOPS™ compounds), an entry inhibitor and/or a small molecule immunomodulator. Examples of additional agents include recombinant interferon alpha 2b, IFN-α, PEG-IFN-α-2a, lamivudine, telbivudine, adefovir dipivoxil, clevudine, entecavir, tenofovir alafenamide and tenofovir disoproxil. Examples of NAPs include, but are not limited to, REP 2139 and REP 2165. Exemplary siRNA's that can be used in combination with a compound, or pharmaceutically acceptable salt thereof, provide herein include those described in WO 2021/178885, which is hereby incorporated by reference for the purpose of describing the siRNA compounds provided therein, such as a siRNA selected from SEQ. ID. NO. 1-617 and SEQ. ID. NO. 618.

In some embodiments, a compound, or a pharmaceutically acceptable salt thereof, as described herein can be administered with one or more additional agent(s) together in a single pharmaceutical composition. In some embodiments, a compound, or a pharmaceutically acceptable salt thereof, can be administered with one or more additional agent(s) as two or more separate pharmaceutical compositions. Further, the order of administration of a compound, or a pharmaceutically acceptable salt thereof, as described herein with one or more additional agent(s) can vary.

### EXAMPLES

Additional embodiments are disclosed in further detail in the following examples, which are not in any way intended to limit the scope of the claims.

Table of Abbreviations

| Abbreviation | Meaning |
|---|---|
| h | hour |
| * | single diastereomer, absolute stereochemistry unknown |
| rt | room temperature |
| EA | ethyl acetate |
| CyH | cyclohexane |
| PE | petroleum ether |
| DIPEA | diisopropylethylamine |
| SFC | Supercritical Fluid Chromatography |

### Example 1

### Compound 1

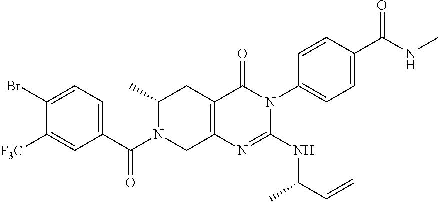 [SMILES: C=C[C@H](C)Nc1nc2c(c(=O)n1-c1ccc(C(=O)NC)cc1)C[C@@H](C)N(C(=O)c1ccc(Br)c(C(F)(F)F)c1)C2]

To a solution of oxalyl dichloride (181 g, 1.43 mol) in CH₂Cl₂ (1.5 L) at −65° C. was added DMSO (111 mL) in CH₂Cl₂ (500 mL). After stirring for 1 h, t-butyl (R)-(1-hydroxypropan-2-yl)carbamate (250 g, 1.43 mol) in CH₂Cl₂ (500 mL) was added dropwise. After stirring for 2 h, Et₃N (144 g, 1.43 mol, 198 mL) was added dropwise. The mixture was gradually warmed to 25° C. and then stirred at 25° C. for 4 h. The reaction was quenched by the addition of NH₄Cl (sat., aq., 2.5 L), and then extracted with CH₂Cl₂ (2×2.5 L). The combined organic layers were dried over Na₂SO₄, the solids were removed by filtration and the filtrate was concentrated under reduced pressure to give the crude product as a colorless oil, t-butyl (R)-(1-oxopropan-2-yl)carbamate (450 g, 2.60 mol, 91% yield) which was used in the next step without further purification.

To a solution of t-butyl (R)-(1-oxopropan-2-yl)carbamate (225 g, 1.30 mol) in CH₂Cl₂ (2.25 L) was added (carbethoxymethylene)triphenylphosphorane (429 g, 1.23 mol). The mixture was stirred at 25° C. for 12 h. The mixture was concentrated under reduced pressure to give the crude product that was purified by silica gel column chromatography (PE:EA=15:1 to 5:1) to afford ethyl (R)-4-((t-butoxycarbonyl)amino)pent-2-enoate (500 g, 2.06 mol, 79.1% yield) as a colorless oil. ¹H NMR (400 MHz, CDCl₃) δ 6.86 (dd, J=15.76, 4.88 Hz, 1H) 5.89 (dd, J=15.70, 1.56 Hz, 1H) 4.58 (br s, 1H) 4.39 (br s, 1H) 4.18 (q, J=7.13 Hz, 2H) 1.44 (s, 9H) 1.24-1.29 (m, 6H).

To a solution of ethyl (R)-4-((t-butoxycarbonyl)amino)pent-2-enoate (125 g, 513 mmol) in CH₃OH (1.25 L) was added 10% Pd/C (6.00 g) and Pd(OH)₂ (6.06 g) under N₂. The suspension was degassed under vacuum and purged with H₂ (1.04 g, 514 mmol) several times. The mixture was stirred under H₂ (50 psi) at 50° C. for 12 h. The solids were removed by filtration under N₂, and the filtrate was evaporated to dryness to afford ethyl (R)-4-((t-butoxycarbonyl)amino)pentanoate (480 g, 1.96 mol, 95% yield) as a colorless oil. ¹H NMR (400 MHz, CDCl₃) δ 4.29-4.45 (m, 1H) 4.13 (q, J=7.13 Hz, 2H) 3.57-3.75 (m, 1H) 2.35 (t, J=7.69 Hz, 2H) 1.66-1.84 (m, 3H) 1.43 (s, 9H) 1.25 (t, J=7.13 Hz, 3H) 1.14 (d, J=6.50 Hz, 3H).

To a solution of ethyl (R)-4-((t-butoxycarbonyl)amino)pentanoate (480 g, 1.96 mol) in HCl/EA (4M, 2.5 L). The mixture was stirred at 25° C. for 2 h. The mixture was concentrated under reduced pressure to give ethyl (R)-4-aminopentanoate HCl (450 g, crude) as a yellow oil that was used directly in the next step without purification.

To mixture of ethyl (R)-4-aminopentanoate HCl (225 g, 1.24 mol) in THF (4 L) and H₂O (1 L), was added K₂CO₃ (427 g, 3.10 mol) at 25° C. After addition, the yellow solution was stirred at 25° C. for 30 min. A solution of ethyl 2-bromoacetate (206 g, 1.24 mol, 137 mL) dropwise at 25° C. over 30 min. The yellow solution was stirred at 25° C. for 11 h. The crude product, ethyl (R)-4-((2-ethoxy-2-oxoethyl)amino)pentanoate (400 g, 1.73 mol, 70% yield), was obtained as a colorless oil that used in the next step without work up or purification.

A solution of (Boc)₂O (189 g, 865 mmol, 199 mL) was added dropwise into ethyl (R)-4-((2-ethoxy-2-oxoethyl)amino)pentanoate (200 g, 865 mmol) over 30 min. The yellow solution was stirred for 6 h at 25° C., and then pumped onto a filter. The filter cake was washed with EA (1 L), and the filtrate was collected. To the filtrate was added H₂O (3 L). The mixture was extracted with EA (2×5 L). The combined organic layers were washed with brine (2 L) and dried over Na₂SO₄. The solids were removed by filtration. The filtrate was concentrated under reduced pressure to give the crude product, ethyl (R)-4-((t-butoxycarbonyl)(2-ethoxy-2-oxoethyl)amino)pentanoate (400 g, 1.21 mol, 70% yield), as a yellow oil, which used in the next step without purification. ¹H NMR (400 MHz, CDCl₃) δ 4.06-4.22 (m, 4H) 3.54-3.93 (m, 2H) 2.26-2.55 (m, 2H) 1.71 (qd, J=7.48, 3.69 Hz, 2H) 1.45-1.55 (m, 6H) 1.42 (s, 4H) 1.22-1.35 (m, 6H).

To a mixture of ethyl (R)-4-((t-butoxycarbonyl)(2-ethoxy-2-oxoethyl)amino)pentanoate (200 g, 603 mmol) in THF (2 L) was added t-BuOK (135 g, 1.21 mol) at 0° C. under N₂. The yellow mixture was stirred at 25° C. for 12 h under N₂. The reaction was quenched by the addition of aq. citric acid (250 g in 3 L of H₂O) at below 10° C. The mixture was extracted with EA (3×2.5 L). The combined organic layers were washed with brine (2 L×1) and dried over Na₂SO₄. The solids were removed by filtration, and the filtrate was concentrated under reduced pressure. The residue was purified by silica column chromatography (PE:EA=15:1 to 10:1) to afford 1-(t-butyl) 4-ethyl 5-oxo-2-(R)-methyl-3,6-dihydropyridine-1,4(2H)-dicarboxylate (210 g, 736 mmol, 61% yield) as a yellow oil. ¹H NMR (400 MHz, CDCl₃) δ 12.06 (s, 1H) 4.54 (br s, 1H) 4.33 (br d, J=19.39 Hz, 1H) 4.23 (dtt, J=10.62, 7.07, 7.07, 3.63, 3.63 Hz, 2H) 3.64 (br d, J=19.26 Hz, 1H) 2.45-2.55 (m, 1H) 2.18 (d, J=15.63 Hz, 1H) 1.47 (s, 9H) 1.31 (t, J=7.13 Hz, 3H) 1.11 (d, J=6.88 Hz, 3H).

To a solution of 1-(t-butyl) 4-ethyl 5-oxo-2-(R)-methyl-3,6-dihydropyridine-1,4(2H)-dicarboxylate (210 g, 736 mmol) in EA (1 L) was added a solution of HCl:EA (4 M, 2 L) dropwise at 25° C. The mixture was stirred at 25° C. for 3 h, and then concentrated under reduced pressure. The crude product was triturated with EA (500 mL) at 25° C. for 30 min to afford ethyl (R)-5-hydroxy-2-methyl-1,2,3,6-tetrahydropyridine-4-carboxylate HCl (140 g, 631 mmol, 86% yield, 100% purity) as a white solid. ¹H NMR (400 MHz, Methanol-d₄) δ 4.29 (q, J=6.96 Hz, 2H) 3.92-4.01 (m, 1H) 3.77-3.87 (m, 1H) 3.42-3.54 (m, 1H) 2.66-2.76 (m, 1H) 2.23-2.39 (m, 1H) 1.43 (d, J=6.50 Hz, 3H) 1.32 (t, J=7.07 Hz, 3H).

A solution of ethyl (R)-5-hydroxy-2-methyl-1,2,3,6-tetrahydropyridine-4-carboxylate HCl (115 g, 519 mmol), in DMF (1 L) was cooled to 0° C. DIPEA (268 g, 2.08 mol, 361 mL), and T₃P (495 g, 778 mmol, 463 mL, 50% purity) were added. The mixture was stirred at 25° C. for 12 h. The reaction was quenched by the addition water 2 L at 25° C. The mixture was diluted with EA (1.5 L) and extracted with EA (3×1 L). The combined organic layers were washed with brine 500 mL and dried over Na₂SO₄. The solids were removed by filtration, and the filtrate was concentrated under reduced pressure. The residue was purified by silica gel chromatography (0 to 10% EA:PE gradient) to afford ethyl (R)-1-(4-bromo-3-(trifluoromethyl)benzoyl)-5-hydroxy-2-methyl-1,2,3,6-tetrahydropyridine-4-carboxylate (130 g, 259 mmol, 50% yield, 87% purity) as a yellow oil. ¹H NMR (400 MHz, CDCl₃) δ 12.10 (br s, 1H) 7.80 (d, J=8.13 Hz, 1H) 7.74 (d, J=1.88 Hz, 1H) 7.42 (dd, J=8.13, 1.88 Hz, 1H) 4.64-5.30 (m, 1H) 4.19-4.34 (m, 2H) 4.08-4.17 (m, 1H) 3.81 (br dd, J=12.13, 2.75 Hz, 1H) 2.58 (br d, J=14.76 Hz, 1H) 2.24 (br d, J=16.01 Hz, 1H) 1.32 (t, J=7.13 Hz, 3H) 1.25 (br t, J=3.13 Hz, 3H).

To a solution of ethyl (R)-1-(4-bromo-3-(trifluoromethyl)benzoyl)-5-hydroxy-2-methyl-1,2,3,6-tetrahydropyridine-4-carboxylate (90.0 g, 206 mmol) in ethanol (900 mL) was added NH₄OAc (79.5 g, 1.03 mol). The mixture was stirred at 60° C. for 2 h. The mixture was concentrated under reduced pressure, diluted with water (200 mL) and extracted with EA (3×200 mL). The combined organic layers were washed with brine (200 mL) and dried over Na₂SO₄. The solids were removed by filtration, and the filtrate was concentrated under reduced pressure. The residue was purified by silica gel chromatography (0 to 50% EA:PE gradient) to afford ethyl (R)-5-amino-1-(4-bromo-3-(trifluoromethyl)benzoyl)-2-methyl-1,2,3,6-tetrahydropyridine-4-carboxylate (55.0 g, 125 mmol, 61% yield, 99% purity) as a yellow solid. ¹H-NMR (400 MHz, DMSO-d₆) δ 7.98 (d, J=8.13 Hz, 1H) 7.84 (d, J=1.75 Hz, 1H) 7.63 (dd, J=8.19, 1.56 Hz, 1H) 6.74-7.47 (m, 2H) 4.63-4.91 (m, 1H) 4.00-4.08 (m, 2H) 3.80-3.95 (m, 1H) 3.59-3.75 (m, 1H) 2.45 (br d, J=5.75 Hz, 1H) 2.14 (br d, J=1.25 Hz, 1H) 1.06-1.20 (m, 6H).

To a solution of ethyl (R)-5-amino-1-(4-bromo-3-(trifluoromethyl)benzoyl)-2-methyl-1,2,3,6-tetrahydropyridine-4-carboxylate (100 g, 230 mmol) and NMM (102 g, 1.01 mol, 111 mL) in CH₂Cl₂ (1 L) was added SCCl₂ (55.5 g, 483 mmol, 37.0 mL) at 0° C. The mixture was stirred at 0° C. for 1 h. The reaction was quenched by the addition ice-water (100 mL) at 0° C. The mixture was diluted with CH₂Cl₂ (150 mL) and extracted with CH₂Cl₂ (3×500 mL). The combined organic layers were washed with brine (500 mL) and dried over Na₂SO₄. The solids were removed by filtration, and the solvent of the filtrate was concentrated under reduced pressure. The residue was purified by silica gel chromatography (0 to 20% EA:PE gradient) to afford ethyl (R)-1-(4-bromo-3-(trifluoromethyl)benzoyl)-5-isothiocyanato-2-methyl-1,2,3,6-tetrahydropyridine-4-carboxylate (100 g, 163 mmol, 71% yield, 78% purity) as a yellow oil. ¹H-NMR (400 MHz, CDCl₃) δ 7.74 (d, J=8.13 Hz, 1H) 7.66 (d, J=1.75 Hz, 1H) 7.34 (dd, J=8.13, 2.00 Hz, 1H) 4.55-5.18 (m, 1H) 4.14-4.26 (m, 3H) 3.67-3.85 (m, 2H) 2.51-2.70 (m, 1H) 2.31-2.47 (m, 1H) 1.29 (t, J=7.13 Hz, 3H) 1.18 (dd, J=7.00, 3.38 Hz, 4H).

To a solution of ethyl (R)-1-(4-bromo-3-(trifluoromethyl)benzoyl)-5-isothiocyanato-2-methyl-1,2,3,6-tetrahydropyridine-4-carboxylate (100 g, 210 mmol) in CH₃CN (1 L) were added 4-amino-N-methylbenzamide (31.5 g, 210 mmol) and Et₃N (53.0 g, 524 mmol, 72.9 mL). The mixture was stirred at 95° C. for 12 h to obtain a yellow suspension. The mixture was concentrated under reduced pressure. The crude product was triturated with EA (500 mL) at 25° C. for 1 h to afford (R)-4-(7-(4-bromo-3-(trifluoromethyl)benzoyl)-6-methyl-4-oxo-2-thioxo-1,4,5,6,7,8-hexahydropyrido[3,4-d]pyrimidin-3(2H)-yl)-N-methylbenzamide (80.0 g, 119 mmol, 57% yield, 86% purity) as a white solid. ¹H-NMR (400 MHz, DMSO-d₆) δ 8.49-8.57 (m, 1H) 8.02 (br d, J=7.63 Hz, 1H) 7.88 (m, 3H) 7.69 (br d, J=7.63 Hz, 1H) 7.29 (br d, J=8.88 Hz, 1H) 7.25 (br s, 1H) 5.08-5.27 (m, 1H) 4.18-4.35 (m, 1H) 4.05-4.14 (m, 1H) 2.80 (d, J=4.50 Hz, 3H) 2.53-2.62 (m, 1H) 2.17-2.36 (m, 1H) 1.18-1.20 (m, 3H).

To a solution of (R)-4-(7-(4-bromo-3-(trifluoromethyl)benzoyl)-6-methyl-4-oxo-2-thioxo-1,4,5,6,7,8-hexahydropyrido[3,4-d]pyrimidin-3(2H)-yl)-N-methylbenzamide (80.0 g, 138 mmol) in dioxane (880 mL) was added SCCl₂ (31.6 g, 275 mmol, 21.1 mL). The mixture was stirred at 100° C. for 2 h and then concentrated under reduced pressure. The residue was purified by silica gel chromatography (0 to 80% EA:PE gradient) to afford 4-[(6R)-7-[4-bromo-3-(trifluoromethyl)benzoyl]-2-chloro-6-methyl-4-oxo-3H,4H,5H,6H,7H,8H-pyrido[3,4-d]pyrimidin-3-yl]-N-methylbenzamide (49.0 g, 81.5 mmol, 59% yield, 97% purity) as an off-white solid. ¹H-NMR (400 MHz, CD₃OD) δ 7.94-8.03 (m, 3H) 7.90 (d, J=1.75 Hz, 1H) 7.61-7.68 (m, 1H) 7.42-7.54 (m, 2H) 5.02-5.49 (m, 1H) 4.13-4.56 (m, 2H) 2.95 (s, 3H) 2.72-2.86 (m, 1H) 2.56 (br d, J=17.89 Hz, 1H) 1.24-1.38 (m, 3H).

NEt₃ (0.83 mL, 6 mmol) was added to a solution of 4-[(6R)-7-[4-bromo-3-(trifluoromethyl)benzoyl]-2-chloro-6-methyl-4-oxo-3H,4H,5H,6H,7H,8H-pyrido[3,4-d]pyrimidin-3-yl]-N-methylbenzamide (500 mg, 0.86 mmol) and (S)-but-3-en-2-amine (183 mg, 2.57 mmol) in anhydrous CH₃CN (15 mL) under N₂. The mixture was stirred at 110° C. for 30 h, at which point additional (S)-but-3-en-2-amine (183 mg, 2.57 mmol) and NEt₃ (0.83 mL, 6 mmol) were added. The mixture was stirred at 110° C. for 18 h and then evaporated to dryness. The residue was dissolved in EA:isopropanol (85:15), washed with sat. aq. NH₄Cl and brine and dried over Na₂SO₄. The solids were removed by filtration, and the filtrate was evaporated to dryness. The crude mixture was purified by chromatography on silica gel (0 to 10% CH₃OH in CH₂Cl₂) to afford 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (1). ¹H-NMR (600 MHz, DMSO-d₆, 80° C.) δ 1.14 (d, J=7.0 Hz, 3H), 1.20 (d, J=7.0 Hz, 3H), 2.35 (d, J=16.6 Hz, 1H), 2.53-2.60 (m, 1H), 2.84 (d, J=4.7 Hz, 3H), 4.03 (d, J 18.7 Hz, 1H), 4.30-4.75 (m, 3H), 4.95-5.05 (m, 2H), 5.28 (d, J=7.3 Hz, 1H), 5.79-5.87 (m, 1H), 7.36 (d, J=7.6 Hz, 2H), 7.65-7.69 (m, 1H), 7.83-7.86 (m, 1H), 7.96-8.02 (m, 3H), 8.29-8.34 (m, 1H) ppm. LC-MS: (C₂₈H₂₇BrF₃N₅O₃) [M+H]⁺: 618/620.

### Example 2

### Compound 2

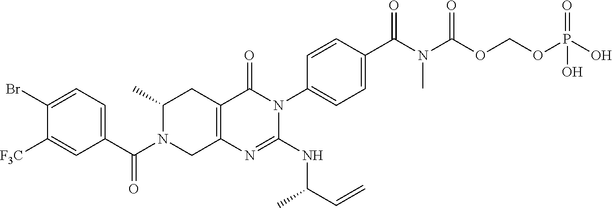 [SMILES: C=C[C@H](C)Nc1nc2c(c(=O)n1-c1ccc(C(=O)N(C)C(=O)OCOP(=O)(O)O)cc1)C[C@@H](C)N(C(=O)c1ccc(Br)c(C(F)(F)F)c1)C2]

LiHMDS 1M in THE (2 eq., 3.23 mL, 3.23 mmol) was added to a solution of compound 1 (1 eq., 1 g, 1.62 mmol) in anhydrous THF (40 mL). The mixture was stirred at −40° C. for 10 min. Chloromethyl chloroformate (1.1 eq., 0.16 mL, 1.78 mmol) was added. The mixture was stirred at −40° C. for 1 h. The reaction was quenched with water and the mixture was extracted with in EA. The combined organic layers were washed with brine, dried over Na₂SO₄ and evaporated. The crude mixture was purified by normal phase chromatography (from 0 to 100% of EA in CyH, then, from 0 to 10% of MeOH in DCM) to afford chloromethyl-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (780 mg, 68%) as a yellow solid. ¹H-NMR (400 MHz, DMSO-d6, 80° C.) δ 1.15 (d, J=7.0 Hz, 3H), 1.20 (d, J=6.8 Hz, 3H), 2.31-2.36 (m, 1H), 2.52-2.59 (m, 1H), 3.30 (s, 3H), 4.04-4.15 (m, 1H), 4.33-4.73 (m, 3H), 4.92-5.04 (m, 2H), 5.08 (d, J=7.9 Hz, 1H), 5.73-5.87 (m, 3H), 7.35-7.42 (m, 2H), 7.65 (dd, J=8.1 Hz, 1.8 Hz, 1H), 7.71-7.77 (m, 2H), 7.83 (d, J=1.9 Hz, 1H), 7.97 (d, J=7.7 Hz, 1H) ppm. LCMS: C₃₀H₂₈BrClF₃N₅O₅: 710/712 [M+H]⁺.

Tetra-n-butylammonium di-tert-butylphosphate (3 eq., 952 mg, 2.11 mmol) was added to a solution of chloromethyl-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1 eq., 500 mg, 0.703 mmol) in THE (50 mL). The mixture was stirred at 40° C. for 16 h. The mixture was then evaporated to dryness and purified by reverse phase chromatography (from 5 to 100% of MeCN in water (+0.1%) of formic acid)) to afford ((di-tert-butoxyphosphoryl)oxy)methyl (4-((R)-7-(4-bromo-3-(trifluoromethyl)benzoyl)-2-C((S)-but-3-en-2-yl)amino)-6-methyl-4-oxo-5,6,7,8-tetrahydropyrido[3,4-d]pyrimidin-3(4H)-yl)benzoyl)(methyl)carbamate (450 mg) as a light-yellow solid. ¹H-NMR (400 MHz, DMSO-d6, 80° C.) δ 1.16 (d, J=6.8 Hz, 3H), 1.21 (d, J=6.71 Hz, 3H), 1.43 (s, 18H), 2.32-2.38 (m, 1H), 2.52-2.60 (m, 1H), 3.32 (s, 3H), 3.98-4.10 (m, 1H), 4.33-4.72 (m, 3H), 4.93-5.05 (m, 2H), 5.14 (d, J=8.0 Hz, 1H), 5.43-5.51 (m, 2H), 5.78-5.87 (m, 11H), 7.35-7.42 (m, 2H), 7.67 (dd, J=8.1 Hz, 1.9 Hz, 1H), 7.70-7.75 (m, 2H), 7.85 (d, J=1.8 Hz, 1H), 7.98 (d, J=8.1 Hz, 1H) ppm. LCMS: C₃₈H₄₆BrF₃N₅O₉P: 886 [M+H]⁺.

TFA (20 eq., 0.76 mL, 10.17 mmol) was added to a solution of (4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrolidin-3(41-1)-yl)benzoyl)(methyl)carbamate (1 eq., 450 mg, 0.509 mmol) in DCM (35 mL) at 0° C. The mixture was stirred at rt for 1 h. The mixture was evaporated to dryness and purified by reverse phase chromatography (from 5 to 100% of MeCN in water (+0.1% of formic acid)) to afford (phosphonooxy)methyl (4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (295 mg, 75%) as a white solid. ¹H-NMR (400 MHz, DMSO-d6, 80° C.) δ 1.17 (d, J=6.9 Hz, 3H), 1.21 (d, J=6.9 Hz, 3H), 2.31-2.38 (m, 1H), 2.52-2.60 (m, 11H), 3.31 (s, 3H), 4.00-4.10 (m, 1H), 4.33-4.72 (m, 3H), 4.93-5.08 (m, 2H). 5.28-5.36 (m, 1H), 5.39-5.51 (m, 2H), 5.78-5.90 (m, 1H), 7.30-7.40 (m, 2H), 7.66 (dd. J=8.0 Hz, 1.7 Hz, 1H), 7.70-7.75 (m, 2H), 7.85 (d, J=1.7 Hz, 1H), 7.98 (d, J=8.1 Hz, 1H) ppm. LCMS: C₃₀H₃₀BrF₃N₅O₉P: 772/774 [M+H]⁺.

### Example 3

### Compound 3

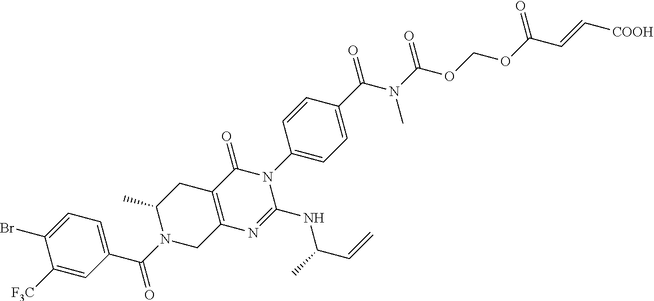 [SMILES: C=C[C@H](C)Nc1nc2c(c(=O)n1-c1ccc(C(=O)N(C)C(=O)OCOC(=O)/C=C/C(=O)O)cc1)C[C@@H](C)N(C(=O)c1ccc(Br)c(C(F)(F)F)c1)C2]

NaI (4 eq., 253 mg, 1.69 mmol), Et₃N (4 eq., 0.23 mL, 1.69 mmol) and (E)-4-(tert-Butoxy)-4-oxobut-2-enoic acid (3 eq., 217 μg, 1.27 mmol) were added to a solution of chloromethyl-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1 eq., 500 mg, 0.703 mmol) in acetone (50 mL). The mixture was stirred at 40° C. for 16 h. The mixture was evaporated to dryness and purified by reverse phase chromatography (from 5 to 100% of MeCN in water (+0.1% of formic acid)) to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl tert-butyl fumarate (220 mg, 62%) as a white solid.

¹H-NMR (400 MHz, DMSO-d6, 80° C.) δ 1.16 (d, J=6.8 Hz, 3H). 1.21 (d, J=6.8 Hz, 31H), 1.45 (s, 9H), 2.31-2.40 (m, 1H), 2.52-2.60 (m, 1H), 3.29 (s, 3H), 4.05 (d, J=18.4 Hz, 1H), 4.22-4.80 (m, 3H), 4.91-5.07 (m, 2H), 5.14 (d, J=8.1 Hz, 1H), 5.66-5.76 (m, 2H), 5.77-5.89 (m, 1H), 6.56-6.76 (m, 2H), 7.35 (d, J=7.8 Hz, 2H), 7.66 (dd, J=7.9 Hz, 1.2 Hz, 1H), 7.69-7.75 (m, 2H), 7.84 (d, J=1.6 Hz, 1H), 7.99 (d, J=8.2 Hz, 1H) ppm. LCMS: C₃₈H₃₉BrF₃N₅O₉: 846/848 [M+H]⁺.

A solution of (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl tert-butyl fumarate (1 eq., 180 mg, 0.21 mmol) in formic acid (9 mL) was stirred at rt for 20 h, evaporated to dryness and co-evaporated with Et₂O to remove formic acid to afford (E)-4-((((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(41H)-yl)benzoyl)(methyl)carbamoyl)oxy)methoxy)-4-oxobut-2-enoic acid (141 mg, 84%) as a white solid. ¹H-NMR (400 MHz, DMSO-d6, 80° C.) δ 1.16 (d, J=6.7 Hz, 3H), 1.21 (d, J=6.7 Hz, 3H), 2.31-2.39 (m, 11H), 2.52-2.62 (m, 1H), 3.30 (s, 3H), 4.05 (d, J=18.2 Hz, 1H), 4.23-4.74 (m, 3H), 4.93-5.06 (m, 2H), 5.14 (d, J=7.9 Hz, 1H), 5.71-5.78 (m, 2H), 5.78-5.90 (m, 1H), 6.59-6.70 (m, 1H), 6.70-6.79 (m, 1H), 7.36 (d, J=8.1 Hz, 2H), 7.67 (dd, J=8.0 Hz, 1.6 Hz, 1H), 7.70-7.76 (m, 2H), 7.85 (d, J=1.3 Hz, 1H), 7.98 (d, J=8.3 Hz, 1H) ppm. LCMS: C₃₄H₃₁BrF₃N₅O₉: 790/792 [M+H]⁺.

### Example 4

### Compound 4

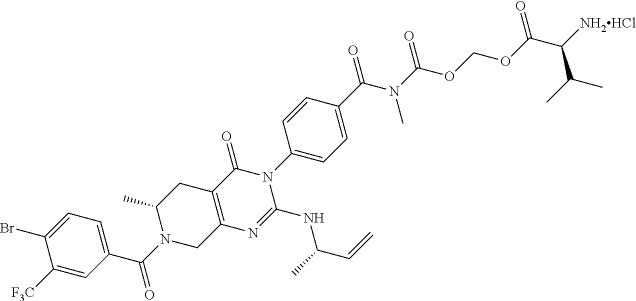

NaI (4 eq., 328 mg, 2.19 mmol), Et₃N (4 eq., 0.31 mL, 2.19 mmol) and Bloc-L-valine (3 eq., 357 mg, 1.65 mmol) were added to a solution of chloromethyl-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(41)-yl)benzoyl)(methyl)carbamate (1 eq., 390 mg, 0.55 mmol) in acetone (30 mL). The mixture was stirred at 40° C. for 16 h. The mixture was evaporated to dryness and purified by reverse phase chromatography (from 5 to 100% of MeCN in water (+0.1% of formic acid)) to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl (tert-butoxycarbonyl)-L-valinate (305 mg, 62%) as a white solid. ¹H-NMR (400 MHz, DMSO-d6, 80° C.) δ 0.89 (dd, J=6.9 Hz, 1.3 Hz, 6H), 1.16 (d, J=6.5 Hz, 3H), 1.21 (d, J=6.5 Hz, 3H), 1.39 (s, 9H), 1.96-2.06 (m, 1H), 2.31-2.39 (m, 1H), 2.53-2.62 (m, 1H), 3.28 (s, 3H), 3.85-3.92 (m, 1H), 3.99-4.11 (m, 1H), 4.27-4.76 (m, 3H), 4.95-5.06 (m, 2H), 5.08-5.13 (m, 1H), 5.62-5.70 (m, 2H), 5.77-5.89 (m, 11H), 6.79-6.91 (m, 1H), 7.38 (d, J=8.4 Hz, 2H), 7.64-7.74 (m, 3H), 7.85 (d, J=1.6 Hz, 1H), 7.99 (d, J=8.1 Hz, 1H) ppm. LCMS: C₄₀H₄₆BrF₃N₆O₉: 891/893 [M+H]⁺.

HCl 4 N in dioxane (40 eq., 3.08 mL, 12.11 mmol) was added to (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl (tert-butoxycarbonyl)-L-valinate (1 eq., 270 mg, 0.303 mmol). The mixture was stirred at rt for 1 h. The mixture was evaporated to dryness to afford the crude mixture which was triturated with Et₂O to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl L-valinate hydrochloride (225 mg, 90%) as a white solid. ¹H-NMR (400 MHz, DMSO-d6, 80° C.) δ 0.98 (dd, J=9.3 Hz, 6.9 Hz, 6H), 1.16 (d, J=6.8 Hz, 3-1), 1.22 (d, J=6.6 Hz, 3H), 2.18-2.26 (m, 1H), 2.31-2.39 (m, 1H), 2.54-2.62 (m, 1H), 3.30 (s, 3H), 3.91 (d, J=4.9 Hz, 1H), 4.01-4.12 (m, 1H), 4.28-4.73 (m, 3H), 4.93-5.05 (m, 2H), 5.15-5.23 (m, 1H), 5.73-5.87 (m, 3H), 7.39 (d, J=7.9 Hz, 2H), 7.67 (dd, J=8.3 Hz, 1.9 Hz, 1H), 7.72-7.77 (m, 2H), 7.84 (d, J=1.8 Hz, 1H), 7.99 (d, J=8.2 Hz, 1H), 8.53 (br.s., 3H) ppm. LCMS: C₃₅H₃₉BrClF₃N₆O₇: 791/793 [M+H]⁺.

### Example 5

### Compound 5

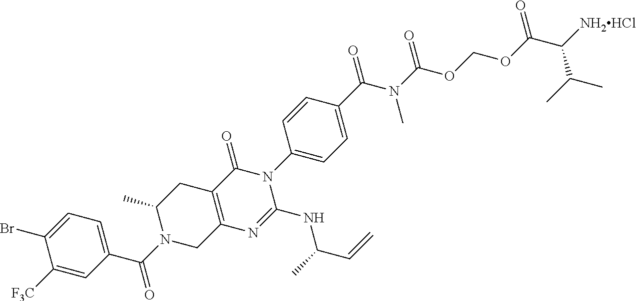

NaI (4 eq., 421.68 mg, 2.81 mmol), Et₃N (4 eq., 0.39 mL, 2.81 mmol) and boc-D-Val-OH (3 eq., 458.41 mg, 2.11 mmol) were added to a solution of chloromethyl-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1 eq., 500 mg, 0.703 mmol) in acetone (50 mL). The mixture was stirred at 60° C. for 3 h. The reaction was quenched with water and extracted with in EA. The combined organic layers were washed with sat. NaHCO₃ and brine, dried over Na₂SO₄ and evaporated. The crude mixture was purified by reverse phase chromatography (from 5 to 100% of MeCN in water (+0.1% of formic acid)) to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl (tert-butoxycarbonyl)-D-valinate (310 mg, 49%) as a white solid. ¹H-NMR (400 MHz, DMSO-d6, 80° C.) δ 0.89 (dd, J=6.9 Hz, 1.3 Hz, 6H), 1.16 (d, J=6.5 Hz, 3H), 1.21 (d, J=6.5 Hz, 3H), 1.39 (s, 9H), 1.96-2.06 (m, 1H), 2.31-2.39 (m, 1H), 2.53-2.62 (m, 1H), 3.27 (s, 3H), 3.85-3.92 (m, 1H), 3.99-4.11 (m, 1H), 4.27-4.76 (m, 3H), 4.95-5.06 (m, 2H), 5.08-5.13 (m, 1H), 5.62-5.70 (m, 2H), 5.76-5.88 (m, 1H), 6.75-6.96 (m, 1H), 7.38 (d, J=8.4 Hz, 2H), 7.67 (dd, J=8.5 Hz, 1.6 Hz, 1H), 7.71 (d, J=8.3 Hz, 2H), 7.85 (d, J=1.2 Hz, 1H), 7.98 (d, J=8.2 Hz, 1H) ppm. LCMS: C₄₀H₄₆BrF₃N₆O₉: 891/893 [M+H]⁺.

HCl 4 N in dioxane (40 eq., 0.45 mL, 1.79 mmol) was added to (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl-(tert-butoxycarbonyl)-D-valinate (1 eq., 40 mg, 0.045 mmol). The mixture was stirred at rt for 1 h. The mixture was evaporated to dryness to afford the crude mixture which was triturated with Et₂O to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl D-valinate hydrochloride (33 mg, 89%) as a white solid. ¹H-NMR (400 MHz, DMSO-d6, 80° C.) δ 0.98 (d, J=7.0 Hz, 3H), 1.00 (d, J=7.0 Hz, 3H), 1.16 (d, J=6.8 Hz, 3H), 1.21 (d, J=6.6 Hz, 3H), 2.18-2.26 (m, 1H), 2.31-2.39 (m, 1H), 2.54-2.62 (m, 1H), 3.29 (s, 3H), 3.91 (d, J=4.9 Hz, 1H), 4.01-4.10 (m, 1H), 4.30-4.72 (m, 3H), 4.93-5.05 (m, 2H), 5.15-5.23 (m, 1H), 5.71-5.89 (m, 3H), 7.39 (d, J=7.9 Hz, 2H), 7.67 (dd, J=8.3 Hz, 1.9 Hz, 1H), 7.72-7.77 (m, 2H), 7.84 (d, J=1.7 Hz, 1H), 7.99 (d, J=8.1 Hz, 1H), 8.53 (br.s., 3H) ppm. LCMS: C₃₅H₃₉BrClF₃N₆O₇: 791/793 [M+H]⁺.

### Example 6

### Compounds 6 and 7

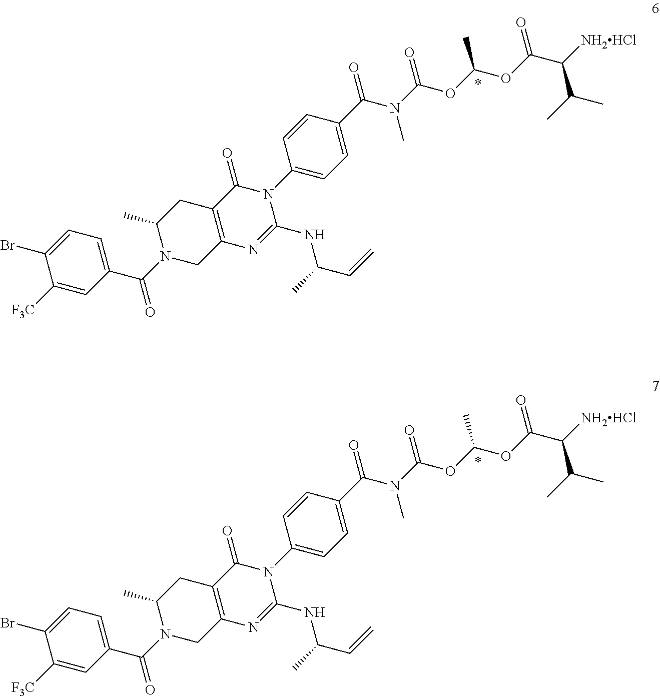

LiHMDS 1M in THF (2 eq., 4.69 mL, 4.69 mmol) was added to a solution of 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (1 eq., 1.45 g, 2.34 mmol) in anhydrous THF (25 mL), and the mixture was stirred at −40° C. for 10 min. 1-chloroethyl chloroformate (1.1 eq., 0.28 mL, 2.58 mmol) was then added and the mixture was stirred at −40° C. for 1 h. The mixture was allowed to warm to rt and stirred for 16 h. The reaction was quenched with water and extracted with in EA. The combined organic layers were washed with brine, dried over Na₂SO₄ and evaporated. The crude mixture was purified by normal phase chromatography (from 0 to 100% of EA in CyH, then, from 0 to 10% of MeOH in DCM) to afford 1-chloroethyl (4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1.1 g, 65%) as a yellow solid. ¹H-NMR (400 MHz, DMSO-d₆, 80° C.) δ 1.14 (d, J=6.7 Hz, 3H), 1.20 (d, J=6.7 Hz, 3H), 1.47 (d, J=5.8 Hz, 3H), 2.28-2.37 (m, 1H), 2.51-2.60 (m, 1H), 3.29 (s, 3H), 3.97-4.06 (m, 1H), 4.28-4.73 (m, 3H), 4.92-5.08 (m, 3H), 5.74-5.87 (m, 1H), 6.38-6.45 (m, 1H), 7.31-7.44 (m, 2H), 7.65 (dd, J=8.0 Hz, 1.8 Hz, 1H), 7.70-7.75 (m, 2H), 7.83 (d, J=1.8 Hz, 1H), 7.97 (d, J=8.5 Hz, 1H) ppm. LCMS: C₃₁H₃₀BrClF₃N₅O₅: 724/726 [M+H]⁺.

NaI (4 eq., 700 mg, 4.69 mmol), Et₃N (4 eq., 0.65 mL, 4.69 mmol) and Boc-L-valine (3 eq., 760 mg, 3.52 mmol) were added to a solution of 1-chloroethyl (4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1 eq., 850 mg, 1.17 mmol) in acetone (60 mL) and the mixture was stirred at 80° C. for 16 h. The mixture was evaporated to dryness and purified by normal phase chromatography (from 0 to 100% of EA in CyH, then, from 0 to 10% of MeOH in DCM), then further purified by SFC (isocratic MeOH/CO₂: 20:80) to obtain the two diastereoisomers (*S)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl (tert-butoxycarbonyl)-L-valinate (6a, 301 mg, 28%) and (*R)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl (tert-butoxycarbonyl)-L-valinate (7a, 290 mg, 27%) as white solids.

Compound 6a: ¹H-NMR (400 MHz, DMSO-d₆, 80° C.) δ 0.86 (d, J=6.6 Hz, 6H), 1.11-1.16 (m, 3H), 1.17-1.22 (m, 6H), 1.36 (s, 9H), 1.93-2.02 (m, 1H), 2.30-2.38 (m, 1H), 2.51-2.58 (m, 1H), 3.25 (s, 3H), 3.78-3.84 (m, 1H), 3.97-4.08 (m, 1H), 4.33-4.68 (m, 3H), 4.92-5.11 (m, 3H), 5.75-5.85 (m, 1H), 6.60-6.67 (m, 1H), 6.79 (br.s., 1H), 7.33-7.42 (m, 2H), 7.64 (dd, J=8.1 Hz, 1.7 Hz, 1H), 7.69-7.74 (m, 2H), 7.82 (d, J=1.7 Hz, 1H), 7.97 (d, J=8.2 Hz, 1H) ppm. LCMS: C₄₁H₄₈BrF₃N₆O₉: 905/907 [M+H]⁺.

Compound 7a: ¹H-NMR (400 MHz, DMSO-d₆, 80° C.) δ 0.83-0.89 (m, 6H), 1.11-1.16 (m, 3H), 1.17-1.22 (m, 6H), 1.36 (s, 9H), 1.93-2.02 (m, 1H), 2.30-2.38 (m, 1H), 2.51-2.58 (m, 1H), 3.25 (s, 3H), 3.78-3.84 (m, 1H), 3.97-4.08 (m, 1H), 4.33-4.68 (m, 3H), 4.92-5.11 (m, 3H), 5.75-5.85 (m, 1H), 6.60-6.67 (m, 1H), 6.74 (br.s., 1H), 7.33-7.42 (m, 2H), 7.64 (dd, J=8.1 Hz, 1.7 Hz, 1H), 7.66-7.72 (m, 2H), 7.83 (d, J=1.7 Hz, 1H), 7.97 (d, J=8.2 Hz, 1H) ppm. LCMS: C₄₁H₄₈BrF₃N₆O₉: 905/907 [M+H]⁺.

HCl 4 N in dioxane (40 eq., 2.87 mL, 11.48 mmol) was added to (*S)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl (tert-butoxycarbonyl)-L-valinate (6a, 1 eq., 260 mg, 0.29 mmol) and the mixture was stirred at rt for 1 h. The mixture was evaporated to dryness to afford the crude mixture which was triturated with Et₂O to afford (*S)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl L-valinate hydrochloride (6, 220 mg, 91%) as a white solid. ¹H-NMR (400 MHz, DMSO-d₆, 80° C.) δ 0.97 (dd; J=6.8 Hz, 5.3 Hz, 6H), 1.16 (d, J=7.0 Hz, 3H), 1.21 (d, J=7.0 Hz, 3H), 1.29 (d, J=5.1 Hz, 3H), 2.15-2.26 (m, 1H), 2.30-2.39 (m, 1H), 2.52-2.61 (m, 1H), 3.30 (s, 3H), 3.87 (d, J=4.4 Hz, 1H), 3.99-4.12 (m, 1H), 4.32-4.77 (m, 3H), 4.91-5.07 (m, 2H), 5.15-5.29 (m, 1H), 5.74-5.86 (m, 1H), 6.72-6.78 (m, 1H), 7.40 (d, J=7.9 Hz, 2H), 7.67 (dd, J=8.5 Hz, 1.6 Hz, 1H), 7.71-7.77 (m, 2H), 7.84 (d, J=1.7 Hz, 1H), 7.99 (d, J=8.2 Hz, 1H), 8.50 (s, 3H) ppm. LCMS: C₃₆H₄₁BrClF₃N₆O₇: 805/807 [M+H]⁺.

HCl 4 N in dioxane (40 eq., 2.09 mL, 8.39 mmol) was added to (*R)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl (tert-butoxycarbonyl)-L-valinate (7a, 1 eq., 190 mg, 0.21 mmol) and the mixture was stirred at rt for 1 h. The mixture was evaporated to dryness to afford the crude mixture which was triturated with Et₂O to afford (*R)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)ethyl L-valinate hydrochloride (7, 150 mg, 85%) as a white solid. ¹H-NMR (400 MHz, DMSO-d₆, 80° C.) δ 0.93-0.99 (m, 6H), 1.14 (d, J=7.0 Hz, 3H), 1.20 (d, J=7.0 Hz, 3H), 1.28 (d, J=5.2 Hz, 3H), 2.12-2.23 (m, 1H), 2.27-2.39 (m, 1H), 2.52-2.59 (m, 1H), 3.28 (s, 3H), 3.81-3.88 (m, 1H), 3.99-4.09 (m, 1H), 4.32-4.77 (m, 3H), 4.91-5.04 (m, 2H), 5.12-5.21 (m, 1H), 5.74-5.86 (m, 1H), 6.67-6.74 (m, 1H), 7.38 (d, J=7.1 Hz, 2H), 7.63-7.66 (m, 1H), 7.70-7.75 (m, 2H), 7.81-7.86 (m, 1H), 7.95-8.01 (m, 1H), 8.42 (s, 3H) ppm. LCMS: C₃₆H₄₁BrClF₃N₆O₇: 805/807 [M+H]⁺.

### Example 7

### Compound 8

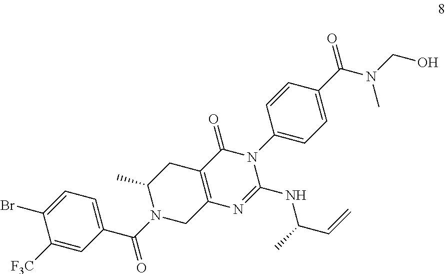 [SMILES: C=C[C@H](C)Nc1nc2c(c(=O)n1-c1ccc(C(=O)N(C)CO)cc1)C[C@@H](C)N(C(=O)c1ccc(Br)c(C(F)(F)F)c1)C2]

Chlorotrimethylsilane (50 eq., 4.13 mL, 32.34 mmol) was added to a solution of 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (1 eq., 400 mg, 0.65 mmol) and paraformaldehyde (1.67 eq., 32.34 mg, 1.08 mmol) in anhydrous THF (8 mL). The mixture was heated to 100° C. for 1 h. The mixture was poured into sat. aq. NaHCO₃ and diluted with EA:iPrOH (85:15). The layers were separated, and the organic layer was dried over Na₂SO₄. The solids were filtered and the filtrate was evaporated to dryness to give a yellow oil, which was purified by reverse phase chromatography (Water (0.5% FA):MeCN from 95:5 to 0:100) to give 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-(hydroxymethyl)-N-methylbenzamide (153 mg, 36%) as a white solid. ¹H-NMR (DMSO-d₆, 400 MHz, 25° C.) δ 1.03-1.27 (m, 6H), 2.16-2.40 (m, 1H), 2.47-2.59 (m, 1H), 3.00 (s, 3H), 3.80-4.38 (m, 2H), 4.53-5.25 (m, 6H), 5.71-5.92 (m, 2H), 5.95-6.32 (m, 1H), 7.23-7.43 (m, 2H), 7.53-7.75 (m, 3H), 7.88 (s, 1H), 8.0 (d, J=8.1 Hz, 1H) ppm. LCMS: C₂₉H₂₉BrF₃N₅O₄: 648/650 [M+H]⁺.

### Example 8

### Compound 9

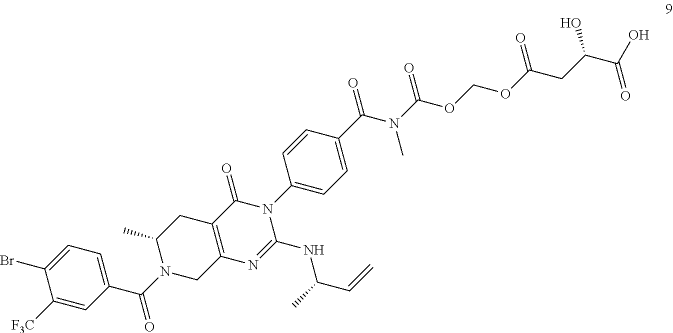 [SMILES: C=C[C@H](C)Nc1nc2c(c(=O)n1-c1ccc(C(=O)N(C)C(=O)OCOC(=O)C[C@H](O)C(=O)O)cc1)C[C@@H](C)N(C(=O)c1ccc(Br)c(C(F)(F)F)c1)C2]

p-TsOH (0.54 g, 3.13 mmol) was added to a mixture of L(−)-malic acid 1 (1 eq., 4.2 g, 31.32 mmol) and 2-methoxypropene (11.58 mL, 125.29 mmol) in acetone (80 mL) at 0° C. After 5 min, the mixture was stirred at 35° C. for 18 h. The mixture was evaporated to dryness, dissolved in EA (50 mL), washed with brine:water (1:1, 4×20 mL) and dried over Na₂SO₄. The solids were removed by filtration and the filtrate was evaporated to afford a black oil, which was treated with cyclohexane to form a precipitate. The precipitate was isolated by filtration. The precipitate was solubilized in a minimum of hot EA (45° C.) and filtered. The filtrate was diluted with cyclohexane to form a precipitate, which was cooled in an ice bath for 1 h. The resulting solids were isolated by filtration and washed with cyclohexane to afford (S)-2-(2,2-dimethyl-5-oxo-1,3-dioxolan-4-yl) acetic acid 2 (2 g, 11.48 mmol, 36%) as a white solid. ¹H-NMR (DMSO-d6, 400 MHz) δ 1.53 (d, J=5.9 Hz, 6H), 2.67-2.81 (m, 2H), 4.79 (dd, J=5.0 Hz, 4.4 Hz, 1H), 12.61 (s, 1H) ppm.

NaI (531 mg, 3.54 mmol), Et₃N (0.49 mL, 3.54 mmol) and (S)-2-(2,2-dimethyl-5-oxo-1,3-dioxolan-4-yl)acetic acid 2 (462 mg, 2.66 mmol) were added to a solution of (4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamate (1 eq., 630 mg, 0.89 mmol) in acetone (10 mL). The mixture was stirred at 65° C. for 16 h. The mixture was then evaporated to dryness and purified by normal phase chromatography (from 0 to 100% of EA in cyclohexane, then, from 0 to 10% CH₃OH in DCM) to afford (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl 2-((S)-2,2-dimethyl-5-oxo-1,3-dioxolan-4-yl)acetate (185 mg, 25%) as a white solid. ¹H-NMR (DMSO-d₆, 400 MHz, 80° C.) δ 1.14 (d, J=6.6 Hz, 3H), 1.20 (d, J=6.9 Hz, 3H), 1.49-1.56 (m, 6H), 2.29-2.37 (m, 1H), 2.52-2.60 (m, 1H), 2.82-2.98 (m, 2H), 3.27 (s, 3H), 3.99-4.09 (m, 1H), 4.24-4.70 (m, 3H), 4.78-4.85 (m, 1H), 4.93-5.04 (m, 2H), 5.08-5.15 (m, 1H), 5.60-5.70 (m, 2H), 5.75-5.89 (m, 1H), 7.32-7.41 (m, 2H), 7.61-7.73 (m, 3H), 7.83 (s, 1H), 7.94-7.99 (m, 1H) ppm. LC-MS: C₃₇H₃₇BrF₃N₅O₁₀: 848/850 [M+H]⁺.

A solution of (((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methyl 2-((S)-2,2-dimethyl-5-oxo-1,3-dioxolan-4-yl)acetate (130 mg, 0.15 mmol) in formic acid (1.3 mL), CH₃OH (1.3 mL) and water (1.3 mL) was stirred at rt for 20 h, and then evaporated to dryness. The residue purified by reverse phase chromatography (5 to 100% CH₃CN in water (+0.1% of formic acid) to afford (S)-4-((((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)methoxy)-2-hydroxy-4-oxobutanoic acid (85 mg, 69%) as a white solid. ¹H-NMR (DMSO-d₆, 400 MHz) δ 1.06-1.32 (m, 6H), 2.22-2.39 (m, 1H), 2.54-2.63 (m, 2H), 2.72-2.84 (m, 1H), 3.26 (s, 3H), 3.75-4.44 (m, 3H), 4.50-5.25 (m, 4H), 5.42-5.96 (m, 4H), 7.25-7.46 (m, 2H), 7.64-7.75 (m, 3H), 7.89 (s, 1H), 7.95-8.03 (m, 1H) ppm. LC-MS: C₃₄H₃₃BrF₃N₅O₁₀: 808/810 [M+H]⁺.

### Example 9

### Compounds 10 and 11

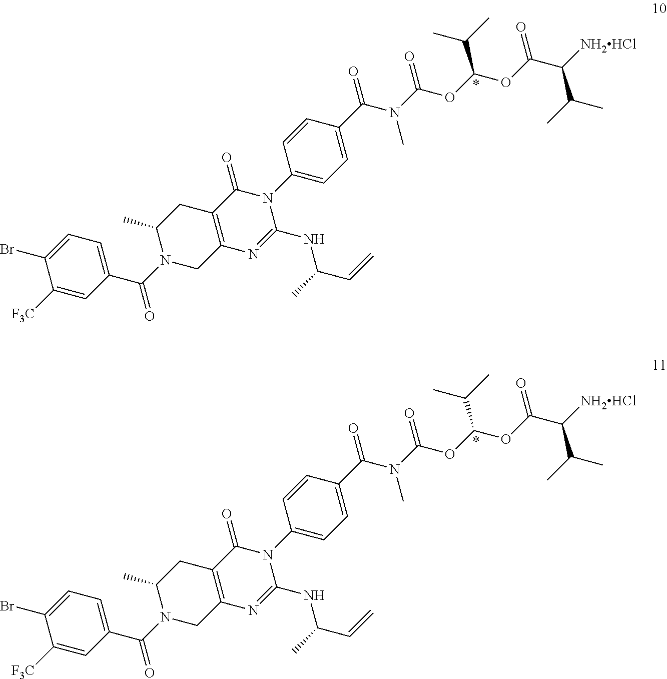

Compounds 10 and 11 were synthetized following the procedure reported for the synthesis of Compounds 6 and 7, using 1-chloro-2-methylpropyl chloroformate in place of 1-chloroethyl chloroformate in the first step.

Compound 10: (*S)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)-2-methylpropyl L-valinate hydrochloride (150 mg, 80%) as a white solid. ¹H-NMR (DMSO-d₆, 400 MHz, 80° C.) δ 0.82 (dd, J=6.9 Hz, 2.4 Hz, 6H), 0.99 (dd, J=7.0 Hz, 3.3 Hz, 6H), 1.16 (d, J=6.7 Hz, 3H), 1.22 (d, J=6.7 Hz, 3H), 1.88-1.97 (m, 1H), 2.19-2.28 (m, 1H), 2.31-2.39 (m, 1H), 2.53-2.62 (m, 1H), 3.30 (s, 3H), 3.94 (d, J=3.8 Hz, 1H), 4.01-4.11 (m, 1H), 4.29-4.74 (m, 3H), 4.95-5.06 (m, 2H), 5.13-5.20 (m, 1H), 5.76-5.87 (m, 1H), 6.58 (d, J=4.2 Hz, 1H), 7.41 (d, J=7.7 Hz, 2H), 7.67 (dd, J=8.0 Hz, 1.8 Hz, 1H), 7.76 (d, J=8.2 Hz, 2H), 7.84 (d, J=1.6 Hz, 1H), 7.99 (d, J=8.2 Hz, 1H), 8.47 (br.s., 3H) ppm. LCMS: C₃₈H₄₅BrClF₃N₆O₇: 833/835 [M+H]⁺.

Compound 11: (*R)-1-(((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)oxy)-2-methylpropyl L-valinate hydrochloride (210 mg, 90%) as a white solid. ¹H-NMR (DMSO-d₆, 400 MHz, 80° C.) δ 0.82 (dd, J=6.7 Hz, 2.4 Hz, 6H), 0.99 (dd, J=7.3 Hz, 6.9 Hz, 6H), 1.14 (d, J=6.7 Hz, 3H), 1.20 (d, J=6.7 Hz, 3H), 1.88-1.97 (m, 1H), 2.19-2.28 (m, 1H), 2.31-2.38 (m, 1H), 2.53-2.61 (m, 1H), 3.29 (s, 3H), 3.84-3.92 (m, 1H), 4.01-4.10 (m, 1H), 4.29-4.71 (m, 3H), 4.95-5.13 (m, 3H), 5.76-5.87 (m, 1H), 6.56 (d, J=4.4 Hz, 1H), 7.38-7.44 (m, 2H), 7.67 (dd, J=8.2 Hz, 1.6 Hz, 1H), 7.75 (d, J=8.7 Hz, 2H), 7.84 (d, J=1.7 Hz, 1H), 7.99 (d, J=8.2 Hz, 1H), 8.52 (br.s., 3H) ppm. LCMS: C₃₈H₄₅BrClF₃N₆O₇: 833/835 [M+H]⁺.

### Example 10

### Compound 12

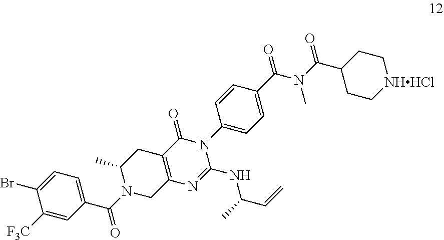

DIPEA (38 mL, 218.1 mmol) was added to a solution of 1-(t-butoxycarbonyl)piperidine-4-carboxylic acid (10 g, 43.62 mmol) and HATU (21.56 g, 56.7 mmol) in anhydrous DMF (160 mL). The mixture was stirred at rt for 1 h and then cooled to 0° C. Methylammonium chloride (3.83 g, 56.7 mmol) was added. The mixture warmed to rt and stirred for 20 h, diluted with EA and washed with 0.25 M HCl, sat. aq. NaHCO₃, water and brine. The organic layer was dried over Na₂SO₄. The solids were filtered and the filtrate was evaporated to dryness to give t-butyl 4-(methylcarbamoyl)piperidine-1-carboxylate (5.66 g, 54%) as a red solid, which was used in the next step without further purification. ¹H-NMR (DMSO-d₆, 400 MHz, 25° C.) δ 1.29-1.44 (m, 11H), 1.56-1.66 (m, 2H), 2.23 (tt, J=11.5 Hz, 3.7 Hz, 1H), 2.55 (d, J=4.6 Hz, 3H), 2.63-2.79 (m, 2H), 3.85-3.99 (m, 2H), 7.65-7.76 (m, 1H) ppm. LC-MS: C₁₂H₂₂N₂O₃: 243 [M+H]⁺.

4-nitrobenzoyl chloride (5.201 g, 28.029 mmol) was added to a solution of t-butyl 4-(methylcarbamoyl)piperidine-1-carboxylate (5.66 g, 23.36 mmol), NEt₃ (4.22 mL, 30.36 mmol) and DMAP (0.29 g, 2.34 mmol) in anhydrous DCM (250 mL) cooled to −78° C. The mixture slowly warmed to rt, then stirred at rt for 20 h. The mixture was diluted with DCM and the reaction was quenched with sat. aq. NaHCO₃. The layers were separated, and the organic layer was washed with brine and dried over Na₂SO₄. The solids were removed by filtration and the filtrate was evaporated to dryness to give t-butyl 4-(methyl(4-nitrobenzoyl)carbamoyl)piperidine-1-carboxylate (9.51 g, crude) as a reddish oil, which was used in the next step without further purification.

Pd/C (0.52 g, 0.49 mmol) was added to a solution of t-butyl 4-(methyl(4-nitrobenzoyl)carbamoyl)piperidine-1-carboxylate (9.51 g, 24.3 mmol) in EA (150 mL) at rt. H₂ was bubbled through the mixture, and the mixture was stirred under H₂ atmosphere for 4 days. The mixture was filtered through packed celite. The filtrate was evaporated to dryness to give a yellow oil, which was purified by chromatography on silica gel (cyclohexane:EA from 100:0 to 50:50) to give a yellow oil. The yellow oil was dissolved in EA and washed with sat. aq. NaHCO₃ and brine. The organic layer was dried over Na₂SO₄. The solids were removed by filtration and the filtrate was evaporated to dryness to afford t-butyl 4-((4-aminobenzoyl)(methyl)carbamoyl)piperidine-1-carboxylate (4.87 g, 55%) as a yellow oil. ¹H-NMR (DMSO-d₆, 400 MHz, 25° C.) δ 1.32-1.49 (m, 11H), 1.62-1.74 (m, 2H), 2.53-2.72 (m, 2H), 2.76-2.88 (m, 1H), 3.04 (s, 3H), 3.81-3.94 (m, 2H), 6.20 (s, 2H), 6.56-6.91 (m, 2H), 7.43-7.48 (m, 2H) ppm. LC-MS: C₁₉H₂₇N₃O₄: 360 [M−H]⁻.

NEt₃ (2.31 mL, 16.6 mmol) was added to a solution of ethyl (R)-1-(4-bromo-3-(trifluoromethyl)benzoyl)-5-isothiocyanato-2-methyl-1,2,3,6-tetrahydropyridine-4-carboxylate (2.64 g, 5.53 mmol) and t-butyl 4-((4-aminobenzoyl)(methyl)carbamoyl)piperidine-1-carboxylate (2 g, 5.53 mmol) in anhydrous MeCN (30 mL). The mixture was stirred at 100° C. for 20 h, cooled to rt and evaporated to dryness to give a solid. The solid was purified by flash chromatography on silica gel (DCM:acetone from 100:0 to 75:25) to afford t-butyl (R)-4-((4-(7-(4-bromo-3-(trifluoromethyl)benzoyl)-6-methyl-4-oxo-2-thioxo-1,4,5,6,7,8-hexahydropyrido[3,4-d]pyrimidin-3(2H)-yl)benzoyl)(methyl)carbamoyl)piperidine-1-carboxylate (2.03 g, 46%) as a yellow solid. ¹H-NMR (DMSO-d₆, 400 MHz, 25° C.) δ 1.08-1.29 (m, 3H), 1.32-1.49 (m, 11H), 1.70-1.81 (m, 2H), 2.19-2.37 (m, 1H), 2.54-2.74 (m, 3H), 2.88-3.00 (m, 1H), 3.15 (s, 3H), 3.80-4.33 (m, 4H), 5.00-5.23 (m, 1H), 7.25-7.42 (m, 2H), 7.65-7.71 (m, 1H), 7.74 (d, J=8.2 Hz, 2H), 7.85-7.94 (m, 1H), 7.98-8.06 (m, 1H), 12.33-13.03 (m, 1H) ppm. LC-MS: C₃₅H₃₇BrF₃N₅O₆S: 792/794 [M+H]⁺.

MeI (0.18 mL, 2.82 mmol) and DBU (0.46 mL, 3.073 mmol) were added to a solution of t-butyl (R)-4-((4-(7-(4-bromo-3-(trifluoromethyl)benzoyl)-6-methyl-4-oxo-2-thioxo-1,4,5,6,7,8-hexahydropyrido[3,4-d]pyrimidin-3(2H)-yl)benzoyl)(methyl)carbamoyl)piperidine-1-carboxylate (2.03 g, 2.56 mmol) in anhydrous DMF (20 mL) cooled at 0° C. The mixture was stirred at 0° C. for 1 h. Water was added and a white precipitate was formed. The suspension was filtered and the solid was washed with water to give t-butyl (R)-4-((4-(7-(4-bromo-3-(trifluoromethyl)benzoyl)-6-methyl-2-(methylthio)-4-oxo-5,6,7,8-tetrahydropyrido[3,4-d]pyrimidin-3(4H)-yl)benzoyl)(methyl)carbamoyl)piperidine-1-carboxylate (2.08 g, 99%, crude) as a yellow solid, which was used as such in the next step without further purification. LC-MS: C₃₆H₃₉BrF₃N₅O₆S: 804/806 [M−H]⁻.

m-CPBA (0.7 g, 2.84 mmol) was added to a solution of t-butyl (R)-4-((4-(7-(4-bromo-3-(trifluoromethyl)benzoyl)-6-methyl-2-(methylthio)-4-oxo-5,6,7,8-tetrahydropyrido[3,4-d]pyrimidin-3(4H)-yl)benzoyl)(methyl)carbamoyl)piperidine-1-carboxylate (2.08 g, 2.58 mmol) in anhydrous DCM (60 mL) at cooled to 0° C. The mixture warmed to rt and stirred for 2 h. The reaction was quenched with sat. aq. Na₂S₂O₃ and diluted with DCM. The layers were separated, and the organic layer was washed with sat. aq. NaHCO₃ and brine, and dried over Na₂SO₄. The solids were removed by filtration and the filtrate was evaporated to dryness to give t-butyl 4-((4-((6R)-7-(4-bromo-3-(trifluoromethyl)benzoyl)-6-methyl-2-(methylsulfinyl)-4-oxo-5,6,7,8-tetrahydropyrido[3,4-d]pyrimidin-3(4H)-yl)benzoyl)(methyl)carbamoyl)piperidine-1-carboxylate (2.31 g, 99%, crude) as a yellow solid, which was used in next step without further purification. LC-MS: C₃₆H₃₉BrF₃N₅O₇S: 820/822 [M−H]⁻.

DIPEA (2.29 mL, 13.87 mmol) was added to a solution of t-butyl 4-((4-((6R)-7-(4-bromo-3-(trifluoromethyl)benzoyl)-6-methyl-2-(methylsulfinyl)-4-oxo-5,6,7,8-tetrahydropyrido[3,4-d]pyrimidin-3(4H)-yl)benzoyl)(methyl)carbamoyl)piperidine-1-carboxylate (1.63 g, 1.98 mmol) and (S)-but-3-en-2-amine hydrochloride (0.64 g, 5.94 mmol) in anhydrous MeCN (10 mL) under N₂. The mixture was stirred at 50° C. for 18 h and then evaporated to dryness to give a dark red solid. The crude mixture was purified by flash chromatography on silica gel (DCM:acetone from 100:0 to 50:50) and then by reverse phase chromatography (Water (0.1% FA):MeCN from 95:5 to 0:100). The collected fractions were combined, extracted with DCM, washed with a mixture of sat. aq. NaHCO₃ and brine, dried over Na₂SO₄ and evaporated to dryness at 30° C. to give t-butyl 4-((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)piperidine-1-carboxylate (270 mg, 16%) as a white solid. ¹H-NMR (DMSO-d₆, 400 MHz, 80° C.) δ 1.17 (d, J=6.8 Hz, 3H), 1.21 (d, J=7.0 Hz, 3H), 1.41 (s, 9H), 1.42-1.60 (m, 2H), 1.81 (d, J=12.6 Hz, 2H), 2.29-2.38 (m, 1H), 2.51-2.61 (m, 1H), 2.64-2.77 (m, 2H), 3.00-3.09 (m, 1H), 3.19 (s, 3H), 3.86-3.97 (m, 2H), 3.99-4.11 (m, 1H), 4.28-4.72 (m, 3H), 4.94-5.05 (m, 2H), 5.34 (d, J=8.3 Hz, 1H), 5.77-5.90 (m, 1H), 7.44 (d, J=7.5 Hz, 2H), 7.61-7.70 (m, 1H), 7.77-7.86 (m, 3H), 7.98 (d, J=8.3 Hz, 1H) ppm. LC-MS: C₃₉H₄₄BrF₃N₆O₆: 829/831 [M+H]⁺.

HCl 4N in dioxane (2.47 mL, 9.88 mmol) was added to t-butyl 4-((4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)(methyl)carbamoyl)piperidine-1-carboxylate (82 mg, 0.099 mmol) at 0° C. The mixture warmed to rt and stirred for 1 h. The crude mixture was cooled to 0° C. Ether was added and a white precipitate was formed. The precipitate was isolated by filtration, and the resulting solid was washed with ether to give a white solid. The solid was triturated ether (2×), DCM (2×) and a mixture of MeCN and ether (2×), and then dried under high vacuum to give N-(4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)benzoyl)-N-methylpiperidine-4-carboxamide hydrochloride (56 mg, 74%) as a white solid. ¹H-NMR (DMSO-d₆, 400 MHz, 80° C.) δ 1.16 (d, J=7.0 Hz, 3H), 1.20 (d, J=6.8 Hz, 3H), 1.78-1.92 (m, 2H), 1.94-2.03 (m, 2H), 2.29-2.38 (m, 1H), 2.50-2.58 (m, 1H), 2.80-2.90 (m, 2H), 3.19 (s, 3H), 3.15-3.33 (m, 3H), 3.97-4.07 (m, 1H), 4.25-4.73 (m, 3H), 4.92-5.05 (m, 2H), 5.36 (d, J=8.0 Hz, 1H), 5.76-5.88 (m, 1H), 7.44 (d, J=8.4 Hz, 2H), 7.65 (dd, J=8.0 Hz, 1.3 Hz, 1H), 7.79-7.86 (m, 3H), 7.97 (d, J=8.6 Hz, 1H), 8.46-8.86 (m, 2H) ppm. LC-MS: C₃₄H₃₇BrClF₃N₆O₄: 729/731 [M+H]⁺.

### Example 15

### Additional Compounds

Additional compounds shown below and including those described herein, can be prepared using similar materials and methods described herein, such as those described herein.

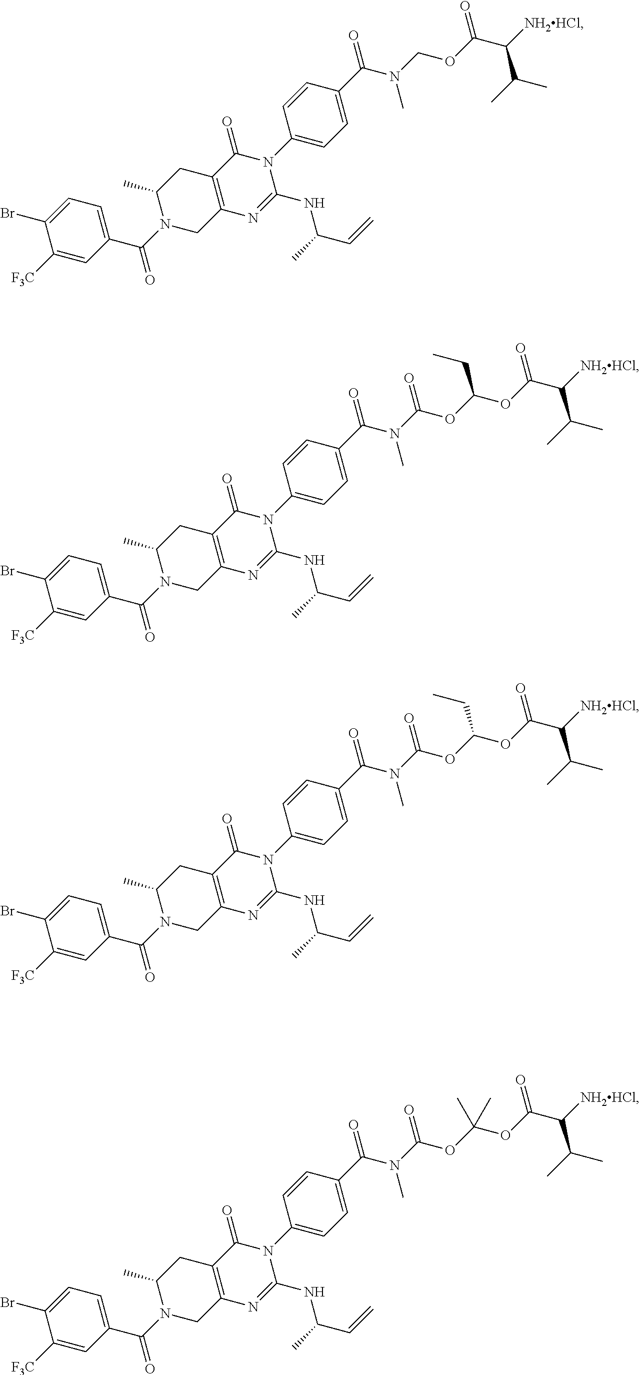

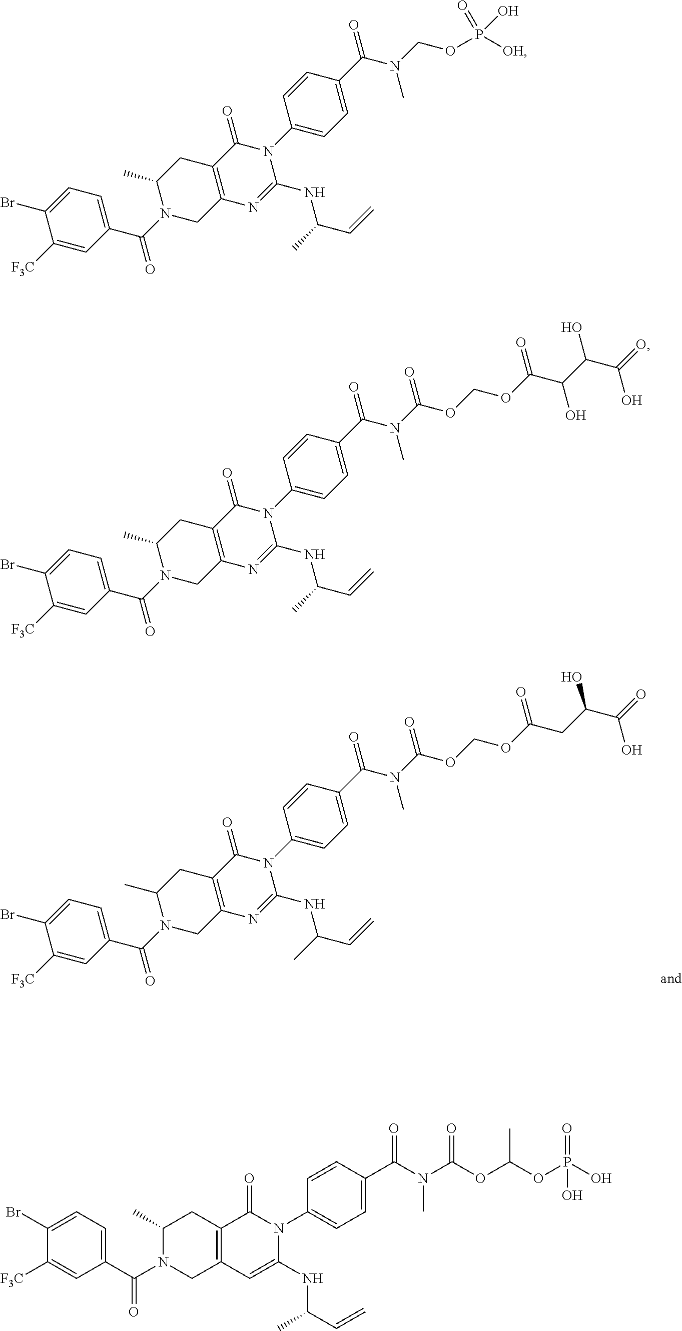 [SMILES: C=C[C@H](C)Nc1nc2c(c(=O)n1-c1ccc(C(=O)N(C)COP(=O)(O)O)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)C(O)C(O)C(=O)O)cc1)C[C@@H](C)N(C(=O)c1ccc(Br)c(C(F)(F)F)c1)C2 ; C=CC(C)Nc1nc2c(c(=O)n1-c1ccc(C(=O)N(C)C(=O)OCOC(=O)C[C@@H](O)C(=O)O)cc1)CC(C)N(C(=O)c1ccc(Br)c(C(F)(F)F)c1)C2 ; C=C[C@H](C)Nc1cc2c(c(=O)n1-c1ccc(C(=O)N(C)C(=O)OC(C)OP(=O)(O)O)cc1)C[C@@H](C)N(C(=O)c1ccc(Br)c(C(F)(F)F)c1)C2]

(including pharmaceutically acceptable salts of any of the foregoing).

### Example A

### Single Dose Pharmacokinetics

Pharmacokinetics parameters were measured after oral dosing in rat. The term 100^{a} refers to an equivalent dose of the parent compound and corresponds to a dose of 128 mg/kg of compound 4. Compound 1 was dosed as a suspension in PEG400/copovidone 95/5 and compound 4 as a suspension in CMC at pH 3. When administered to a subject, the test compounds are absorbed intact and then converted to the parent compound, Compound 1. The results of the single dose pharmacokinetics studies are provided in Table 1. As shown by the results in Table 1, compounds of Formula (I) (including pharmaceutically acceptable salts thereof) have increased concentration in the blood and more reaches systemic circulation compared to the parent compound. Thus, compounds of Formula (I) (including pharmaceutically acceptable salts thereof) have increased oral bioavailability.

TABLE 1

| | Dose | Cmax | AUC | |
|---|---|---|---|---|
| Compound | mg/kg | ng/mL | ng · h/mL | F % |
| 1 | 100 | 856 | 5435 | 6.9 |
| 4 | 100^{a} | 8423 | 89075 | 113 |

### Example B

### HBV-DNA Antiviral Assay Using HepG2.117 Cells

The following assay procedure describes the HBV antiviral assay, using HepG2.117 cells, which carry a stably integrated genotype D HBV genome under the control of a Tet-off promoter, and intracellular HBV DNA quantification as endpoint. Cell viability is assessed in parallel by measuring the intracellular ATP content using CellTiter-Glo 2.0 (Promega).

On day 0, HepG2.117 cells (which are maintained in routine cell culture with doxycycline present in the medium at a final concentration of 1 μg/mL) are seeded in 96-well plates (white with clear bottom) at a density of 2.0×10⁴ cells/well (0.1 mL/well) in medium without doxycycline to induce pgRNA transcription and subsequent formation of HBV particles. The cells are incubated at 37° C. and 5% CO₂.

On day 1, medium is removed from each well, the test articles are diluted in culture medium without doxcycyline and 100 μL was added to cell culture wells (9 concentrations, 4-fold dilution). For each plate, 6 untreated (merely DMSO) wells are included. The final concentration of DMSO in the culture medium is 2%. Each plate is prepared in duplicate (one for HBV DNA extraction, one for CellTiter-Glo 2.0 measurement). The cells are incubated at 37° C. and 5% CO₂ for 3 days.

On day 4, cell viability is assessed using CellTiter-Glo 2.0 and cell lysates are prepared for HBV DNA extraction and subsequent quantification by qPCR.

HBV DNA Quantification by qPCR

Medium is removed from each well and 100 μL of 0.33% NP-40 in H₂O was added to each well. Plates are sealed, incubated at 4° C. for 5 mins, vortexed extensively and centrifuged briefly. Next, 35 μL of lysate is added to 65 μL QuickExtract DNA Extraction Solution (Epicentre) in a PCR plate for each well. PCR plate is incubated at 65° C. for 6 mins, 98° C. for 2 mins and finally cooled to 4° C. HBV DNA is then quantified by qPCR with HBV-specific primers and probes as specified in Table 2 using the Bio-Rad SSOAdvanced Universal Probes Supermix on a CFX96 machine (Bio-Rad). The PCR cycle program consisted of 95° C. for 3 mins, followed by 40 cycles at 95° C. for 10 sec and 60° C. for 30 sec.

TABLE 2
HBV DNA Primers and Probe
for HepG2.117 assay

| Items | Name | Sequence (5′→3′) |
|---|---|---|
| HBV | HBV- | GTGTCTGCGGCGTTTTATCA |
| | forward | (SEQ ID NO.: 1) |
| Primer | HBV- | GACAAACGGGCAACATACCTT |
| | reverse | (SEQ ID NO.: 2) |
| HBV | HBV | FAM/CCTCTKCAT/ZEN/ |
| Probe | probe | CCTGCTGCTATGCCTCATC/ |
| | | 3IABKFQ/ (SEQ ID NO.: 3) |

A DNA standard is prepared by dilution of an IDT gBlock corresponding to the amplicon with concentrations ranging from 10\hat{ }2 to 10\hat{ }8 copies/input (i.e., per 4 μL) and used to generate a standard curve by plotting Cq values vs. HBV DNA standard concentration. The quantity of HBV DNA in each sample is determined by interpolating from the standard curve.

### Cell Viability

Using the other plates, the cell viability is quantified by CellTiter-Glo 2.0 according to the manufacturer's manual. In brief, 100 μL of reagent solution is added to the culture plates and shaken for 2′. The plates are incubated at rt for 10 min and luminescence signal is subsequently measured on a VarioSkan Lux (ThermoFisher) plate reader.

### Data Analysis

Cell viability is calculated as follows: % Cell viability=(luminescence value of test sample)/(average luminescence value of 2% DMSO control)×100%. HBV DNA inhibition was calculated as follows: 100−(HBV DNA copy number of test sample)/(average HBV DNA copy number of 2% DMSO control)×100%. No normalization to entecavir is required due to the excellent dynamic window of this assay. The CC₅₀, EC₅₀ and EC₉₀ values were determined by dose-response curves fitted using non-linear regression.

As shown in Table 3, compounds of Formula (I) are active against HBV, where ‘A’ indicates an EC₅₀≤50 nM, ‘B’ indicates an EC₅₀>50 nM and ≤500 nM, ‘C’ indicates an EC₅₀>500 nM and ≤5000 nM, and ‘D’ indicates an EC₅₀>5000 nM. Cell viability assessments indicated a large window between effective antiviral concentrations and cytotoxic compound concentrations.

TABLE 3

| Compound | EC₅₀ (nM) | CC₅₀ (nM) |
|---|---|---|
| 1 | A | 46791 |
| 2 | A | >50000 |
| 3 | A | >50000 |
| 4 | A | >50000 |
| 5 | A | 48822 |
| 6 | A | >50000 |
| 7 | A | >50000 |
| 8 | A | >50000 |
| 9 | A | >50000 |
| 10 | A | >50000 |
| 11 | A | >50000 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

1. A compound of Formula (I), or a pharmaceutically acceptable salt thereof, having the structure: 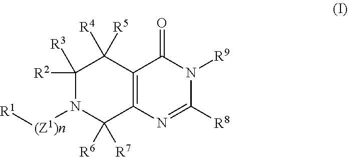
wherein:
n is 0 or 1;
Z¹ is —C(═O)— or —NH—C(═O)—;
R¹ is selected from the group consisting of an optionally substituted aryl, an optionally substituted heteroaryl, an optionally substituted heterocyclyl, an optionally substituted aryl(C₁₋₄ alkyl), an optionally substituted heteroaryl(C₁₋₄ alkyl) and an optionally substituted heterocyclyl(C₁₋₄ alkyl);
R² and R³ are independently hydrogen or an unsubstituted C₁₋₄ alkyl;
R⁴, R⁵, R⁶ and R⁷ are each hydrogen;
R⁸ is —NR^{10A}R^{10B} or an optionally substituted C₂₋₁₂ alkynyl, wherein the C₂₋₁₂ alkynyl is optionally substituted with one or more substituents selected from the group consisting of amino, —NH—C(═O)(an unsubstituted C₁₋₄ alkyl), hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl an unsubstituted 4-6 membered monocyclic heterocyclyl, an optionally substituted aryl and an optionally substituted 5-6 membered monocyclic heteroaryl;
R⁹ is a substituted phenyl, a substituted monocyclic heteroaryl or a substituted fused-bicyclic heteroaryl, wherein the substituted phenyl, the substituted monocyclic heteroaryl or the substituted fused-bicyclic heteroaryl is substituted with —C(═O)NR¹¹R¹² and the substituted phenyl, the substituted monocyclic heteroaryl or the substituted fused-bicyclic heteroaryl is optionally further substituted;
R^{10A} is hydrogen, an unsubstituted C₁₋₆ alkyl, a monocyclic C₃₋₆ cycloalkyl optionally substituted with one or two halogens, an optionally substituted 5-6 membered monocyclic heteroaryl, an optionally substituted 4-6 membered monocyclic heterocyclyl or an optionally substituted monocyclic C₃₋₆ cycloalkyl(C₁₋₄ alkyl);
R^{10B} is selected from the group consisting of an optionally substituted C₂₋₈ alkenyl, an optionally substituted C₂₋₈ alkynyl, an optionally substituted aryl, an optionally substituted aryl(C₁₋₄ alkyl), an optionally substituted heteroaryl(C₁₋₄ alkyl) and an optionally substituted heterocyclyl(C₁₋₄ alkyl), wherein the C₂₋₈ alkenyl and the C₂₋₈ alkynyl is optionally substituted with one or more substituents selected from the group consisting of amino, hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl and an unsubstituted 4-6 membered monocyclic heterocyclyl; and
R¹¹ is H or an unsubstituted C₁₋₄ alkyl;
R¹² is —(C(═O)—O—CR^{13A}R^{13B})p-R¹⁴;
p is 0 or 1;
R^{13A} and R^{13B} are independently H or an unsubstituted C₁₋₄ alkyl; and
R¹⁴ is selected from the group consisting of 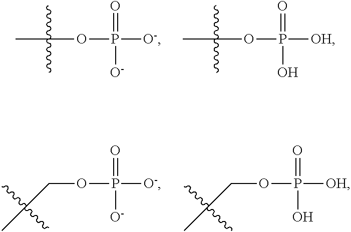
—O-linked α-amino acid, —CH₂—O-linked α-amino acid, a hydroxy-substituted C₁₋₄ alkyl, —O—(C═O)—CH(OH)—CH(OH)—C(═O)OH, —O—(C═O)—CH₂—CH(OH)—C(═O)OH, —O—(C═O)—CH═CH—C(═O)OH and —(C═O)-(an unsubstituted or a substituted monocyclic heterocyclyl).

2. The compound of claim 1, wherein n is 1; and Z¹ is —C(═O)—; and R¹ is an optionally substituted phenyl.

3.-7. (canceled)

8. The compound of claim 2, wherein R¹ is

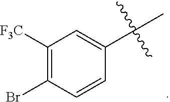 [SMILES: *c1ccc(Br)c(C(F)(F)F)c1]

9.-18. (canceled)

19. The compound of claim 2, wherein R², R⁴, R⁵, R⁶ and R⁷ are each hydrogen; R³ is an unsubstituted C₁₋₄ alkyl.

20. (canceled)

21. The compound of claim 2, wherein R⁸ is —NR^{10A}R^{10B}.

22. The compound of claim 21, wherein R^{10A} is hydrogen; and R^{10B} is an unsubstituted C₂₋₈ alkenyl.

23.-34. (canceled)

35. The compound of claim 2, wherein R⁹ is a substituted phenyl.

36.-39. (canceled)

40. The compound of a claim 35, wherein R¹¹ is H.

41. The compound of claim 35, wherein R¹¹ is an unsubstituted C₁₋₄ alkyl.

42. The compound of claim 41, wherein R¹⁴ is

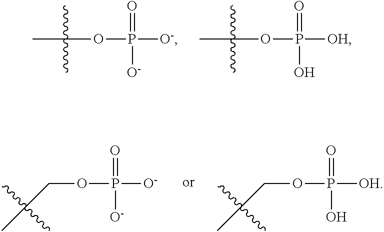

43. (canceled)

44. The compound of claim 41, wherein R¹⁴ is —O-linked α-amino acid, —CH₂—O-linked α-amino acid or —(C═O)— (an unsubstituted or a substituted monocyclic heterocyclyl).

45. The compound of claim 41, wherein R¹⁴ is —O—(C═O)—CH(OH)—CH(OH)—C(═O)OH, —O—(C═O)—CH₂—CH(OH)—C(═O)OH or —O—(C═O)—CH═CH—C(═O)OH.

46.-50. (canceled)

51. The compound of claim 41, wherein p is 1; R^{13A} is H; and R^{13B} is H.

52. The compound of claim 41, wherein R^{13A} is an unsubstituted C₁₋₄ alkyl; and R^{13B} is H or an unsubstituted C₁₋₄ alkyl.

53.-55. (canceled)

56. The compound of claim 1 selected from the group consisting of: 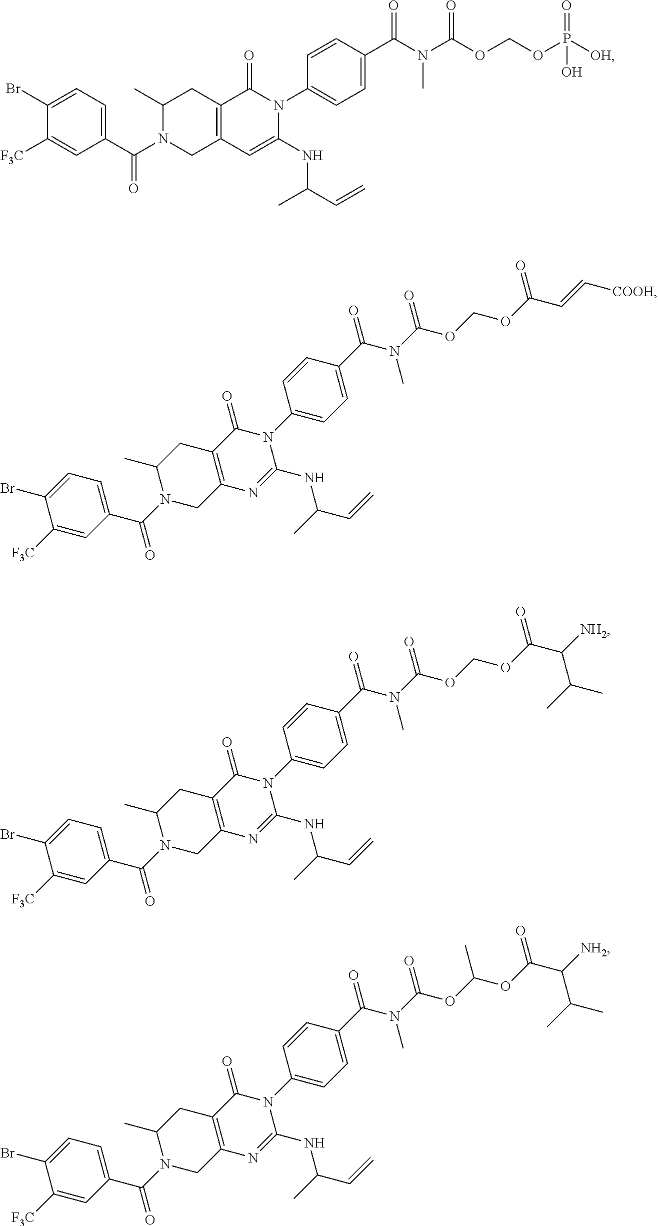 [SMILES: C=CC(C)Nc1cc2c(c(=O)n1-c1ccc(C(=O)N(C)C(=O)OCOP(=O)(O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)/C=C/C(=O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)C(N)C(C)C)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OC(C)OC(=O)C(N)C(C)C)cc1)CC(C)N(C(=O)c1ccc(Br)c(C(F)(F)F)c1)C2] 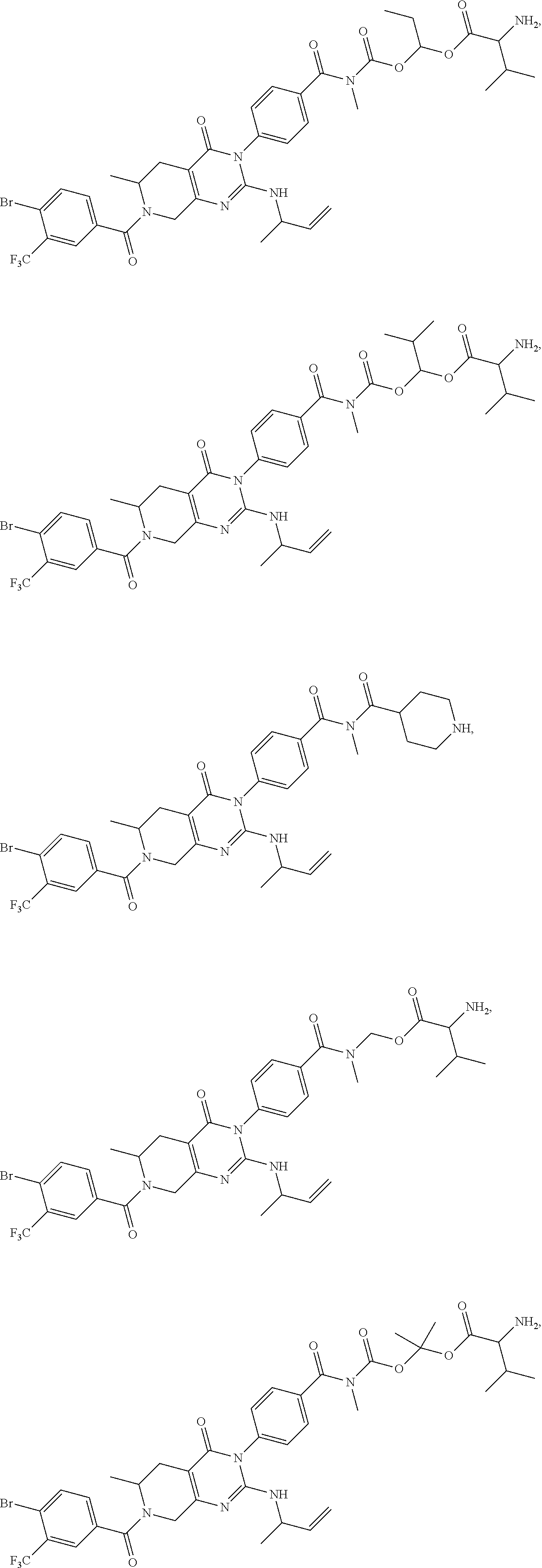 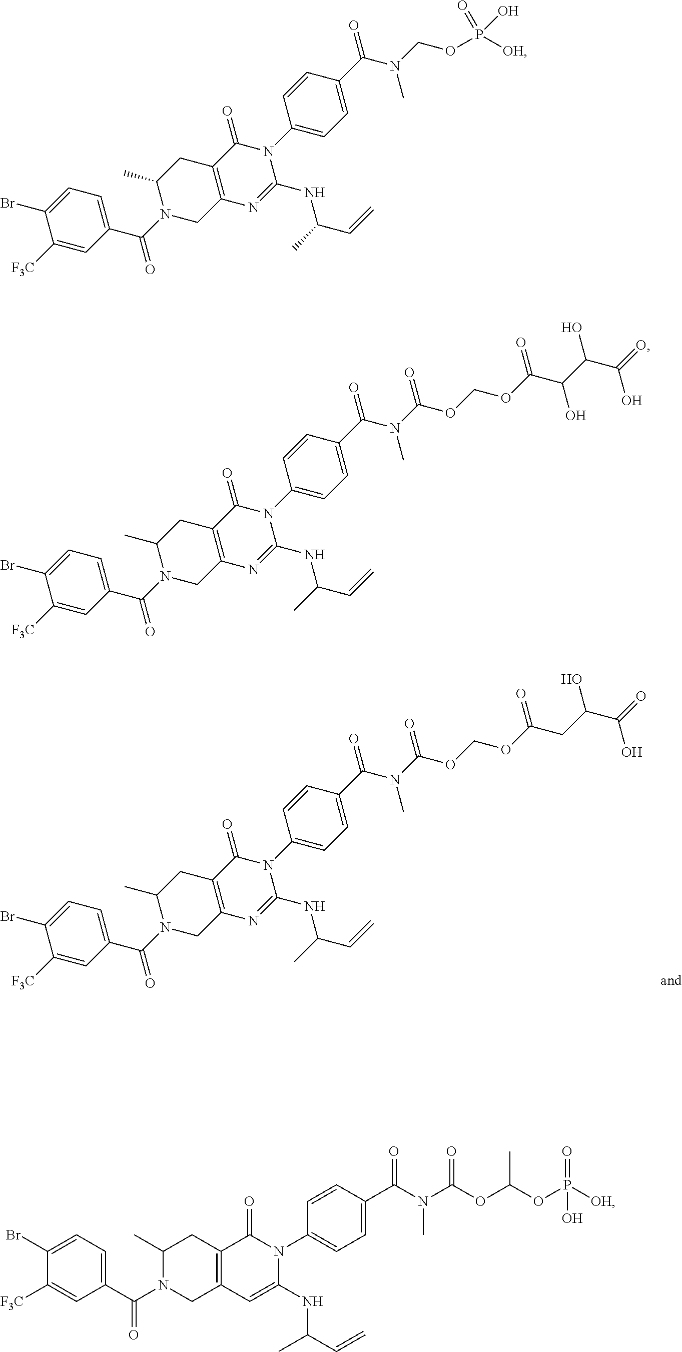 [SMILES: C=C[C@H](C)Nc1nc2c(c(=O)n1-c1ccc(C(=O)N(C)COP(=O)(O)O)cc1)C[C@@H](C)N(C(=O)c1ccc(Br)c(C(F)(F)F)c1)C2 ; C=CC(C)Nc1nc2c(c(=O)n1-c1ccc(C(=O)N(C)C(=O)OCOC(=O)C(O)C(O)C(=O)O)cc1)CC(C)N(C(=O)c1ccc(Br)c(C(F)(F)F)c1)C2 ; C=CC(C)Nc1nc2c(c(=O)n1-c1ccc(C(=O)N(C)C(=O)OCOC(=O)CC(O)C(=O)O)cc1)CC(C)N(C(=O)c1ccc(Br)c(C(F)(F)F)c1)C2 ; C=CC(C)Nc1cc2c(c(=O)n1-c1ccc(C(=O)N(C)C(=O)OC(C)OP(=O)(O)O)cc1)CC(C)N(C(=O)c1ccc(Br)c(C(F)(F)F)c1)C2]
or a pharmaceutically acceptable salt of any of the foregoing.

57. The compound of claim 1 selected from the group consisting of: 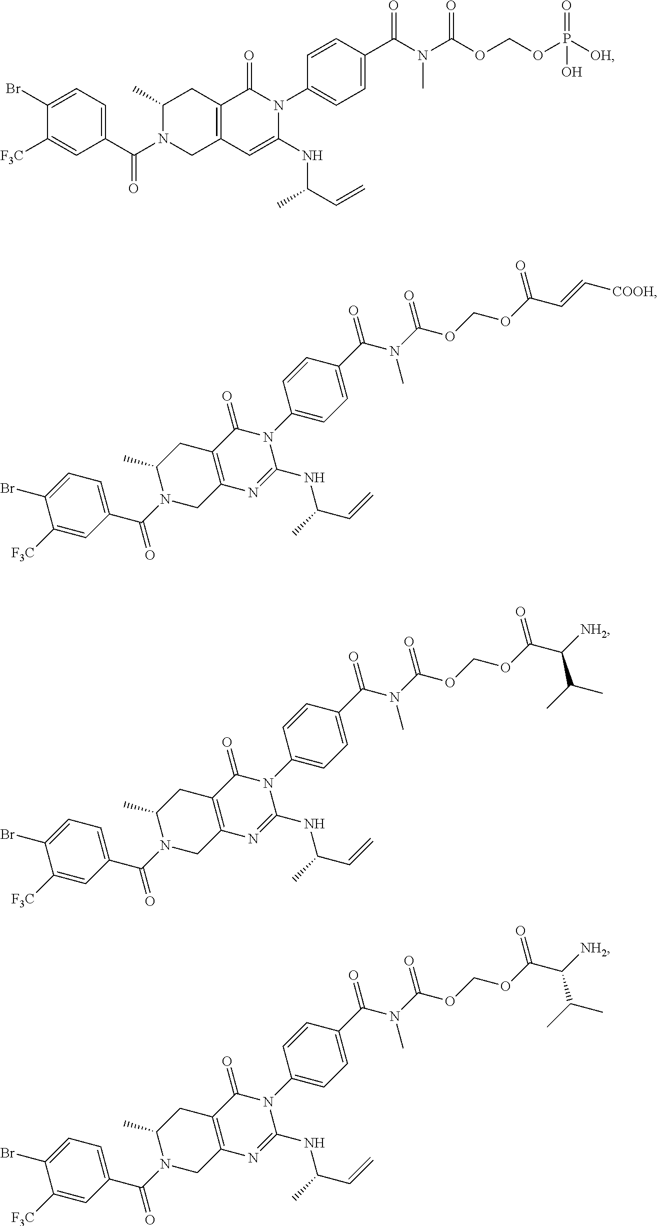 [SMILES: C=C[C@H](C)Nc1cc2c(c(=O)n1-c1ccc(C(=O)N(C)C(=O)OCOP(=O)(O)O)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)/C=C/C(=O)O)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)[C@@H](N)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)OCOC(=O)[C@H](N)C(C)C)cc1)C[C@@H](C)N(C(=O)c1ccc(Br)c(C(F)(F)F)c1)C2] 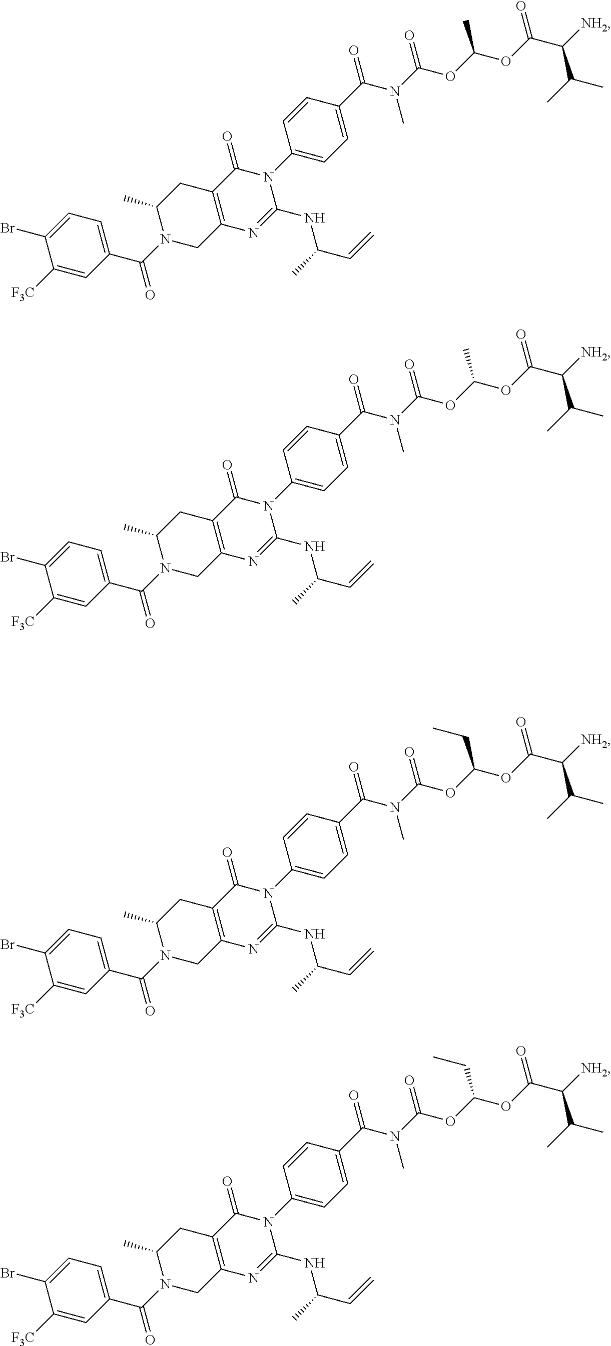 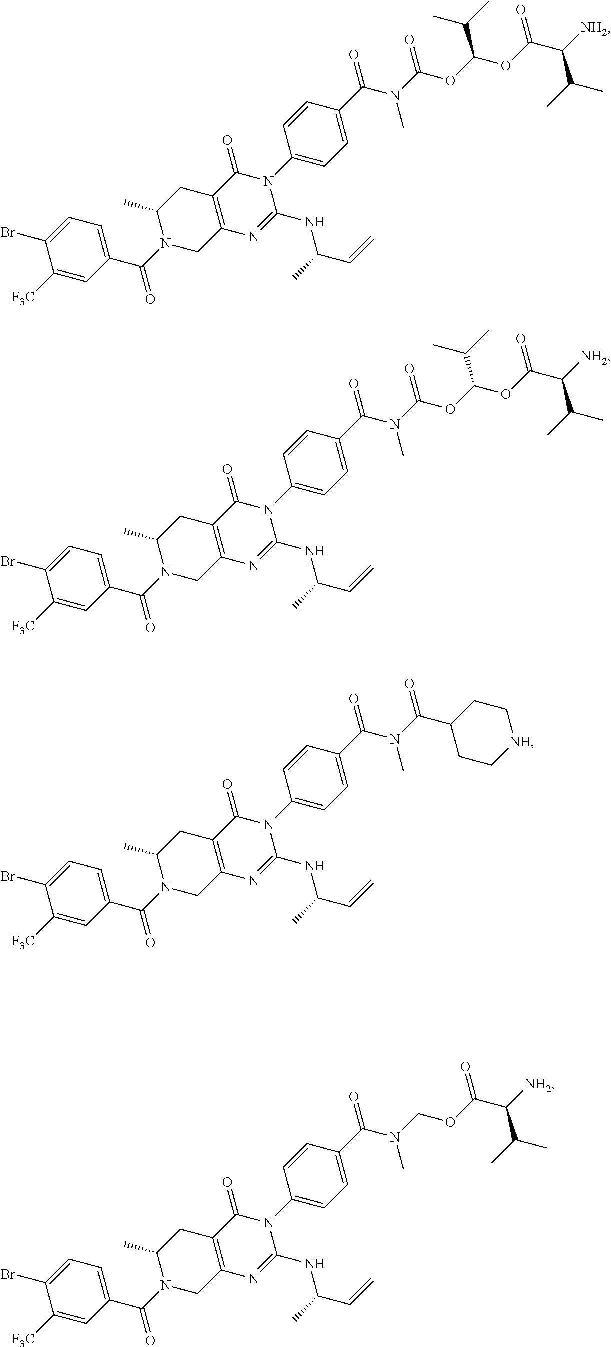 [SMILES: C=C[C@H](C)Nc1nc2c(c(=O)n1-c1ccc(C(=O)N(C)C(=O)O[C@H](OC(=O)[C@@H](N)C(C)C)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)O[C@@H](OC(=O)[C@@H](N)C(C)C)C(C)C)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)C(=O)C3CCNCC3)cc1)C[C@@H](C)N(C(=O)c1ccc(Br)c(C(F)(F)F)c1)C2 ; C=C[C@H](C)Nc1nc2c(c(=O)n1-c1ccc(C(=O)N(C)COC(=O)[C@@H](N)C(C)C)cc1)C[C@@H](C)N(C(=O)c1ccc(Br)c(C(F)(F)F)c1)C2] 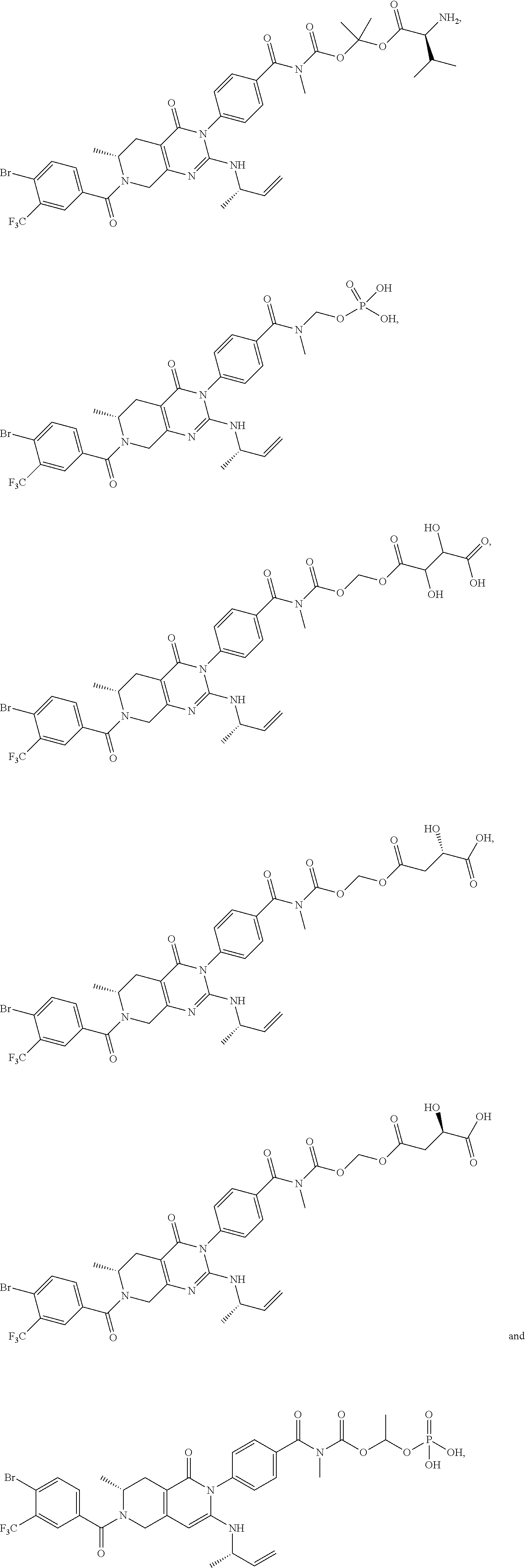
or a pharmaceutically acceptable salt of any of the foregoing.

58. A pharmaceutical composition comprising an effective amount of a compound of claim 1, or a pharmaceutically acceptable salt thereof, and excipient.

59.-66. (canceled)

67. A method for treating hepatitis B in a subject comprising administering to the subject in need thereof an effective amount of a compound of claim 1, or a pharmaceutically acceptable salt thereof, suffering from hepatitis B.

68. The method of claim 67, wherein the compound further treats hepatitis D in the subject.

69. The method of claim 67, further comprising administering an additional agent selected from the group consisting of an interferon, a nucleoside analog, a nucleotide analog, a sequence specific oligonucleotide, a nucleic acid polymer, an entry inhibitor and a small molecule immunomodulator.

70. (canceled)


SEQUENCE LISTING (3 sequences)

SEQ ID NO: 1 - DNA, 20 bp, synthetic construct
  misc_feature 1..20: note = Synthetic HBV-forward primer
  source 1..20: mol_type = other DNA
gtgtctgcggcgttttatca

SEQ ID NO: 2 - DNA, 21 bp, synthetic construct
  misc_feature 1..21: note = Synthetic HBV-reverse primer
  source 1..21: mol_type = other DNA
gacaaacgggcaacatacctt

SEQ ID NO: 3 - DNA, 28 bp, synthetic construct
  misc_feature 1..28: note = Synthetic HBV-probe
  modified_base 1: mod_base = OTHER; note = n=Fluorescein-c
  modified_base 9: mod_base = OTHER; note = n=t-ZEN quencher
  modified_base 28: mod_base = OTHER; note = n=c-3' Iowa Black FQ
  source 1..28: mol_type = other DNA
nctctkcancctgctgctatgcctcatn